(19) United States
(12) Patent Application Publication
(10) Pub. No.: US 2024/0150330 A1
(43) Pub. Date: May 9, 2024

(54) CHROMANS AND BENZOFURANS AS 5-HT1A AND TAAR1 AGONISTS

(71) Applicant: Sunovion Pharmaceuticals Inc., Marlborough, MA (US)

(72) Inventors: Dario DOLLER, Sparta, NJ (US); Hongbing HUANG, Woodbridge, CT (US); Linghong XIE, Southborough, MA (US)

(73) Assignee: Sunovion Pharmaceuticals Inc., Marlborough, MA (US)

(21) Appl. No.: 18/554,214

(22) PCT Filed: Apr. 6, 2022
(86) PCT No.: PCT/US2022/071563

§ 371 Date: Oct. 6, 2023

Related U.S. Application Data
(60) Provisional application No. 63/173,371, filed on Apr. 10, 2021.

(51) Int. Cl. C07D 405/04; C07D 311/04; C07D 413/04; C07D 417/04

(52) U.S. Cl. CPC C07D 405/04; C07D 311/04; C07D 413/04; C07D 417/04

(57) ABSTRACT

Provided herein are compounds, pharmaceutical compositions, and methods of use thereof, including methods of treating a neurological or psychiatric disease or disorder. For example, provided herein is a compound of Formula I:

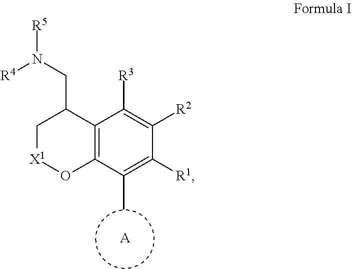

or a pharmaceutically acceptable salt thereof, wherein values for the variables (e.g., Ring A, X¹, R¹, R², R³, R⁴, and R⁵) are as disclosed herein. The compounds disclosed herein (e.g., compounds of Formula I, or pharmaceutically acceptable salts thereof) and pharmaceutical compositions can be used to treat a neurological or psychiatric disease or disorder.

### CROSS REFERENCE TO RELATED APPLICATIONS

This application claims priority to U.S. Provisional Application No. 63/173,371, filed Apr. 10, 2021, the entire disclosure of which is hereby incorporated herein by reference.

### FIELD

The present disclosure relates to compounds, pharmaceutical compositions, and methods of use thereof, including methods of treating a neurological or psychiatric disease or disorder.

### BACKGROUND

Treatments for neurological or psychiatric diseases and disorders typically target certain neurotransmitter sites. For example, the D₂ dopamine receptor has been a primary target for both typical and atypical antipsychotic agents used to treat a variety of neurological or psychiatric diseases or disorders, including schizophrenia, bipolar disorder, and as an adjunctive to antidepressants for treating major depressive disorder. Wang et al. NATURE 555, 269-273 (2018). However, drugs that target the D₂ dopamine receptor can cause serious or potentially life-threatening side effects. Wang et al. NATURE 555, 269-273 (2018). Despite decades of research on non-D₂ mechanisms of action, developing non-D₂ therapies that are both safe and effective has been challenging. Girgis et al., J. PSYCHIATRIC RES. 108, 57-83 (2019). Accordingly, there is a need for novel therapeutic agents for treating neurological and psychiatric diseases and disorders.

### SUMMARY

In one aspect, the present disclosure provides compounds of Formula I

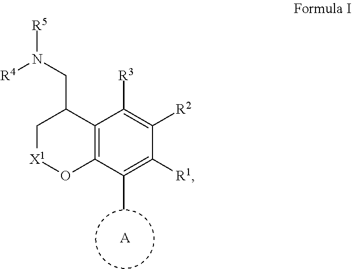

or a pharmaceutically acceptable salt thereof,
wherein:
Ring A is (C₅-C₇)aryl or 5 to 7 membered heteroaryl comprising 1 to 3 heteroatoms;
   wherein each (C₅-C₇)aryl or 5 to 7 membered heteroaryl comprising 1 to 3 heteroatoms is independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl;
X¹ is a bond or CH₂;
R¹, R², and R³ are each independently H, halogen, (C₁-C₄)alkyl, (C₂-C₄)alkenyl, (C₂-C₄)alkynyl, (C₁-C₄)alkoxy, or (C₁-C₄)haloalkyl; and
R⁴ and R⁵ are independently H, (C₁-C₄)alkyl, or (C₃-C₆)cycloalkyl.

In another aspect, the invention relates to a pharmaceutical composition comprising a compound disclosed herein, or a pharmaceutically acceptable salt thereof, and a pharmaceutically acceptable excipient.

In another aspect, the invention relates to a method for treating a neurological or psychiatric disease or disorder in a subject, comprising administering to said subject an effective amount of a compound, or a pharmaceutically acceptable salt thereof, or pharmaceutical composition disclosed herein.

### DETAILED DESCRIPTION

A description of example embodiments follows.

Provided herein are definitions to assist with interpreting this disclosure. Whenever appropriate, terms used in the singular will also include the plural. Unless the context clearly indicates otherwise, terms used herein have the following meanings.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

As used herein, the term “heteroatom” refers to nitrogen (N), oxygen (O) or sulfur (S) atoms, in particular nitrogen or oxygen. When one heteroatom is S, it can be optionally mono- or di-oxygenated (i.e., —S(O)— or —S(O)₂). Unless otherwise indicated, any heteroatom with unsatisfied valences is assumed to have hydrogen atoms sufficient to satisfy the valences.

As used herein, the term “alkyl” refers to a branched or straight-chain, monovalent, hydrocarbon group having the specified number of carbon atoms, and the general formula CₙH₂ₙ₊₁. Thus, the term “(C₁-C₆)alkyl” refers to a branched or straight-chain, monovalent, hydrocarbon group of the general formula CₙH₂ₙ₊₁ wherein n is 1, 2, 3, 4, 5 or 6. Examples of alkyl include, but are not limited to, methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like.

As used herein, the term “alkenyl” refers to an aliphatic group containing at least one carbon-carbon double bond and having from 2 to 4 carbon atoms (i.e., C₂-C₄ alkenyl). Examples of alkenyl groups include ethenyl, propenyl, butadienyl (including 1,2-butadienyl, and 1,3-butadienyl).

As used herein, the term “alkynyl” refers to aliphatic group containing at least one carbon-carbon triple bond and having from 2 to 4 carbon atoms (i.e., C₂-C₄ alkynyl). The term “alkynyl” also includes those groups having one triple bond and one double bond.

The term “alkoxy,” as used herein, refers to an alkyl group attached through an oxygen linking atom, wherein alkyl is as described herein. “(C₁-C₆)alkoxy” refers to an alkoxy group in which a (C₁-C₆)alkyl is attached through an oxygen linking atom. Examples of alkoxy groups include, but are not limited to, methoxy, ethoxy, propoxy (e.g., n-propoxy and iso-propoxy), and butoxy (e.g., t-butoxy).

“Halogen” and “halo,” as used herein, refer to a fluoro, chloro, bromo or iodo substituent. In some embodiments, halogen refers to a fluoro, chloro or bromo substituent. In some embodiments, halogen refers to a fluoro or chloro substituent. In some embodiments, halogen refers to a chloro, bromo or iodo substituent. In some embodiments, halogen refers to a chloro or bromo substituent.

“Haloalkyl,” as used herein, refers to an alkyl group wherein one or more hydrogen atoms is each independently replaced by a halogen, wherein alkyl is as described herein. “Haloalkyl” includes mono-, poly- and perhaloalkyl groups. “(C₁-C₆)haloalkyl” refers to a (C₁-C₆)alkyl wherein one or more hydrogen atoms is each independently replaced by a halogen. Examples of haloalkyl include, but are not limited to, fluoromethyl, difluoromethyl, trifluoromethyl, trichloromethyl, pentafluoroethyl, pentachloroethyl, 2,2,2 trifluoroethyl, heptafluoropropyl, and heptachloropropyl.

“Haloalkoxy,” as used herein, refers to a haloalkyl group attached through an oxygen linking atom, wherein haloalkyl is as described herein. “(C₁-C₆)haloalkoxy” refers to a haloalkoxy group in which a (C₁-C₆)haloalkyl is attached through an oxygen linking atom. Examples of haloalkoxy include, but are not limited to, trifluoromethoxy, difluoromethoxy, 2,2,2 trifluoroethoxy, and pentafluoroethoxy.

“Cyano” or “—CN” as used herein, means —C≡N.

The term “substituted,” as used herein, means that at least one (e.g., one, two, three, four, five, six, etc., from one to five, from one to three, one or two) hydrogen atom is replaced with a non-hydrogen substituent, provided that normal valencies are maintained and that the substitution results in a stable compound. Unless otherwise indicated, an “optionally substituted” group can have a substituent at each substitutable position of the group and, when more than one position in any given structure may be substituted with more than one substituent selected from a specified group, the substituent can be the same or different at every position. Alternatively, an “optionally substituted group” can be unsubstituted.

In cases wherein there are nitrogen atoms on compounds of the present disclosure, these nitrogen atoms may be converted to N-oxides by treatment with an oxidizing agent (e.g., mCPBA and/or hydrogen peroxide) to afford other compounds of this disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 substituents, then said group may be unsubstituted or substituted with up to three substituents, and each substituent is selected independently from the other substituent(s).

Combinations of substituents and/or variables are permissible only if such combinations result in stable compounds.

As a person of ordinary skill in the art would understand, for example, a ketone (—C(H)C(O)) group in a molecule may tautomerize to its enol form (—C═C(OH)). This disclosure is intended to cover all possible tautomers even when a structure depicts only one of them.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., a compound of Formula I, a subformula of a compound of Formula I, such as a compound of Formula II(A) or III(A)), as well as isomers, such as stereoisomers (including diastereoisomers, enantiomers and racemates), geometrical isomers, conformational isomers (including rotamers and atropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts.

In some embodiments, the recitation of a “compound” is intended to encompass both free compounds and pharmaceutically acceptable salts thereof. As a convention, the phrase “or a pharmaceutically acceptable salt thereof” is explicitly recited when the structural formula of the compound is explicitly recited, but no difference in inclusion or exclusion of pharmaceutically acceptable salts is thereby intended.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present disclosure.

As used herein, the term “isomers” refers to different compounds that have the same molecular formula but differ in arrangement and configuration of the atoms. “Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers can be designated using the conventional RS system (e.g., (1S,2S)); a single stereoisomer with known relative configuration but unknown absolute configuration is designated with stars (e.g., (R*), (S*), (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, and which are not mirror-images of each other. The absolute stereochemistry can be specified according to the Cahn-Ingold-Prelog R-S system. When a compound is a pure enantiomer, the stereochemistry at each chiral carbon may be specified by either R or S. Resolved compounds can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC. Alternatively, graphic representations of racemic, ambiscalemic and scalemic or enantiomerically pure compounds used herein are a modified version of the denotations taken from Maehr J. Chem. Ed. 62, 114-120 (1985): simple lines provide no information about stereochemistry and convey only connectivity; solid and broken wedges are used to denote the absolute configuration of a chiral element; solid and broken bold lines indicated relative stereochemistry of indeterminate absolute configuration. For example, the graphic representation:

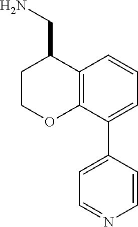 [SMILES: NC[C@@H]1CCOc2c(-c3ccncc3)cccc21]

indicates an enantiomer, that is, either of the two representations below:

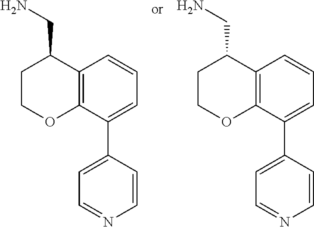 [SMILES: NC[C@@H]1CCOc2c(-c3ccncc3)cccc21 ; NC[C@H]1CCOc2c(-c3ccncc3)cccc21]

in any ratio, and likewise,

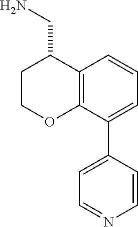 [SMILES: NC[C@H]1CCOc2c(-c3ccncc3)cccc21]

N is the other enantiomer of

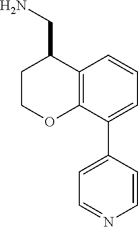 [SMILES: NC[C@@H]1CCOc2c(-c3ccncc3)cccc21]

and is either of the two representations below:

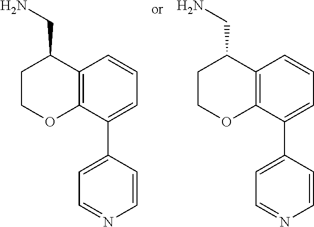 [SMILES: NC[C@@H]1CCOc2c(-c3ccncc3)cccc21 ; NC[C@H]1CCOc2c(-c3ccncc3)cccc21]

in any ratio, while the representation:

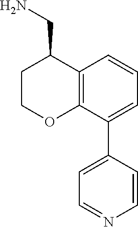 [SMILES: NC[C@@H]1CCOc2c(-c3ccncc3)cccc21]

indicates a single enantiomer with the absolute configuration depicted, e.g., (R)-(8-(pyridin-4-yl)chroman-4-yl)methanamine in the illustration above.

The “enantiomeric excess” or “% enantiomeric excess” of a composition can be calculated using the equation shown below. In the example shown below, a composition contains 90% of one enantiomer, e.g., the S enantiomer, and 10% of the other enantiomer, e.g., the R enantiomer. In this example, % ee=(90-10)/100=80%.

Thus, a composition containing 90% of one enantiomer and 10% of the other enantiomer is said to have an enantiomeric excess of 80%. Some compositions described herein contain an enantiomeric excess of at least about 50%, 75%, 90%, 95%, or 99% of the S enantiomer. In other words, the compositions contain an enantiomeric excess of the S enantiomer over the R enantiomer. In other embodiments, some compositions described herein contain an enantiomeric excess of at least about 50%, 75%, 90%, 95%, or 99% of the R enantiomer. In other words, the compositions contain an enantiomeric excess of the R enantiomer over the S enantiomer.

For instance, an isomer/enantiomer can, in some embodiments, be provided substantially free of the corresponding enantiomer, and can also be referred to as “optically enriched,” “enantiomerically enriched,” “enantiomerically pure” and “non-racemic,” as used interchangeably herein. These terms refer to compositions in which the percent by weight of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the R enantiomer, such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 85% by weight of one enantiomer relative to other enantiomer, such as at least about 90% by weight, and further such as at least 95% by weight. In certain embodiments, the compound provided herein is made up of at least about 90% by weight of one enantiomer. In other embodiments, the compound is made up of at least about 95%, 98%, or 99% by weight of one enantiomer.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than about 55%, about 60%, about 65%, about 70%, about 75%, about 80%, about 85%, about 90%, about 95%, about 96%, about 97%, about 98%, about 99%, about 99.5%, or more. In other embodiments, the compound mixture has an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

In other embodiments, the compound mixture has an (R)-enantiomeric purity of greater than about 55%, about 60%, about 65%, about 70%, about 75%, about 80%, about 85%, about 90%, about 95%, about 96%, about 97%, about 98%, about 99%, about 99.5% or more. In some other embodiments, the compound mixture has an (R)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In other embodiments, the compound mixture contains identical chemical entities except for their stereochemical orientations, namely (S)- or (R)-isomers. For example, if a compound disclosed herein has —CH(R)— unit, and R is not hydrogen, then the —CH(R)— is in an (S)- or (R)-stereochemical orientation for each of the identical chemical entities. In some embodiments, the mixture of identical chemical entities is a racemic mixture of (S)- and (R)-isomers. In another embodiment, the mixture of the identical chemical entities (except for their stereochemical orientations), contain predominately (S)-isomers or predominately (R)-isomers. For example, the (S)-isomers in the mixture of identical chemical entities are present at about 55%, about 60%, about 65%, about 70%, about 75%, about 80%, about 85%, about 90%, about 95%, about 96%, about 97%, about 98%, about 99%, about 99.5%, or more, relative to the (R)-isomers. In some embodiments, the (S)-isomers in the mixture of identical chemical entities are present at an (S)-enantiomeric excess of greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5% or more.

In another embodiment, the (R)-isomers in the mixture of identical chemical entities (except for their stereochemical orientations), are present at about 55%, about 60%, about 65%, about 70%, about 75%, about 80%, about 85%, about 90%, about 95%, about 96%, about 97%, about 98%, about 99%, about 99.5%, or more, relative to the (S)-isomers. In some embodiments, the (R)-isomers in the mixture of identical chemical entities (except for their stereochemical orientations), are present at a (R)-enantiomeric excess greater than about 55% to about 99.5%, greater than about 60% to about 99.5%, greater than about 65% to about 99.5%, greater than about 70% to about 99.5%, greater than about 75% to about 99.5%, greater than about 80% to about 99.5%, greater than about 85% to about 99.5%, greater than about 90% to about 99.5%, greater than about 95% to about 99.5%, greater than about 96% to about 99.5%, greater than about 97% to about 99.5%, greater than about 98% to greater than about 99.5%, greater than about 99% to about 99.5%, or more.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described herein below), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

Unless otherwise specified, the word “includes” (or any variation thereon, e.g., “include”, “including”, etc.) is intended to be open-ended (and not limited to the examples cited in the text following “includes”). For example, the phrase—“parenteral, including . . . ” as used herein to describe one route of administering a medicament contemplates any route of administering a medicament via injection or infusion, using, for example, but not limited to, a syringe or catheter, which techniques include, but are not limited to, administration via subcutaneous, intravenous, intramuscular, intra-articular, intra-synovial, intrasternal, intrathecal, intrahepatic, intralesional and intracranial injection or infusion techniques.

Unless otherwise specified, the phrase “such as” is intended to be open-ended. For example, “chorea (such as Sydenham's chorea, Huntington's disease, benign hereditary chorea, neuroacanthocytosis, symptomatic chorea, drug-induced chorea and hemiballism)” means that a chorea can be, but is not limited to, Sydenham's chorea, Huntington's disease, benign hereditary chorea, neuroacanthocytosis, symptomatic chorea, drug-induced chorea and hemiballism.

Where one or more ranges are referred to throughout this specification, each range is understood to encompass each discrete point within the range, including the endpoints describing the range, as if the same were fully set forth herein.

In one aspect, the present disclosure provides compounds of Formula I

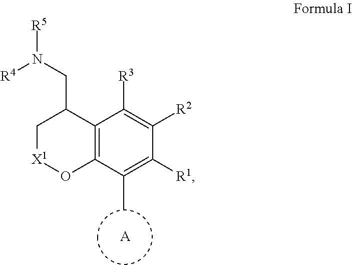

or a pharmaceutically acceptable salt thereof,
wherein:
Ring A is (C₅-C₇)aryl or 5 to 7 membered heteroaryl comprising 1 to 3 heteroatoms;
   wherein each (C₅-C₇)aryl or 5 to 7 membered heteroaryl comprising 1 to 3 heteroatoms is independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl;
X¹ is a bond or CH₂;
R¹, R², and R³ are each independently H, halogen, (C₁-C₄)alkyl, (C₂-C₄)alkenyl, (C₂-C₄)alkynyl, (C₁-C₄)alkoxy, or (C₁-C₄)haloalkyl; and
R⁴ and R⁵ are independently H, (C₁-C₄)alkyl, or (C₃-C₆)cycloalkyl.

In some embodiments, Ring A is (C₅-C₇)aryl independently optionally substituted with 1 to 3 halogen, (C₁-C₄)alkyl, (C₂-C₄)alkenyl, (C₂-C₄)alkynyl, or (C₁-C₄)haloalkyl. In some embodiments, Ring A is 5 to 7 membered heteroaryl comprising 1 to 3 heteroatoms independently optionally substituted with 1 to 3 halogen, (C₁-C₄)alkyl, (C₂-C₄)alkenyl, (C₂-C₄)alkynyl, (C₁-C₄)alkoxy or (C₁-C₄)haloalkyl. In some embodiments, Ring A is phenyl, thiophenyl, pyrrolyl, furanyl, thiazolyl, oxazolyl, imidazolyl, pyridinyl, pyridazinyl, pyrimidinyl, or pyrazinyl, each independently optionally substituted with 1 to 3 halogen, (C₁-C₄)alkyl, (C₂-C₄)alkenyl, (C₂-C₄)alkynyl, (C₁-C₄)alkoxy or (C₁-C₄)haloalkyl. In some embodiments, Ring A is phenyl, thiophenyl, pyrrolyl, furanyl, thiazolyl, oxazolyl, imidazolyl, pyridinyl, pyridazinyl, pyrimidinyl, or pyrazinyl, each independently optionally substituted with 1 to 3 halogen, methyl, ethyl, methoxy, ethoxy, or trifluoromethyl.

In some embodiments, X¹ is a bond. In some embodiments, X¹ is CH₂.

In some embodiments, R¹, R², and R³ are each independently H, halogen, or (C₁-C₄)alkyl. In some embodiments, R¹, R², and R³ are each independently H, fluoro, or methyl.

In some embodiments, R⁴ and R⁵ are each independently H or (C₁-C₄)alkyl. In some embodiments, R⁴ and R⁵ are each independently H or methyl.

In another aspect, the present disclosure provides compounds of Formula II(A)

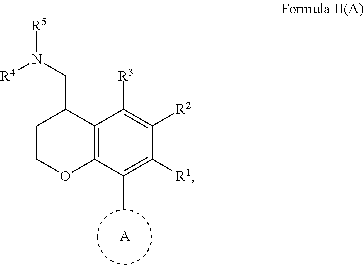

or a pharmaceutically acceptable salt thereof,
wherein Ring A, R¹, R², R³, R⁴, and R⁵ are as defined herein.

In another aspect, the present disclosure provides compounds of Formula III(A)

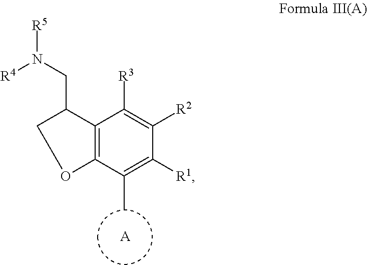

or a pharmaceutically acceptable salt thereof,
wherein Ring A, R¹, R², R³, R⁴, and R⁵ are as defined herein.

In one embodiment, provided is a compound according to Formula I wherein said compound is selected from the following Table 1:

TABLE 1
Compounds of Formula

| | |
|---|---|
| 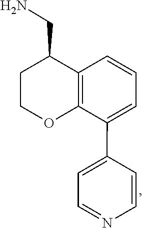 [SMILES: NC[C@@H]1CCOc2c(-c3ccncc3)cccc21] | 1 |
| 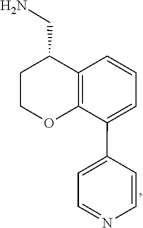 [SMILES: NC[C@H]1CCOc2c(-c3ccncc3)cccc21] | 2 |
| 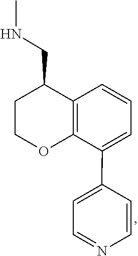 [SMILES: CNC[C@@H]1CCOc2c(-c3ccncc3)cccc21] | 3 |
| 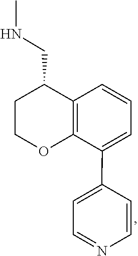 [SMILES: CNC[C@H]1CCOc2c(-c3ccncc3)cccc21] | 4 |
| 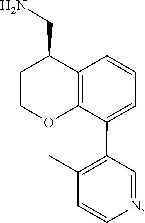 [SMILES: Cc1ccncc1-c1cccc2c1OCC[C@H]2CN] | 5 |
| 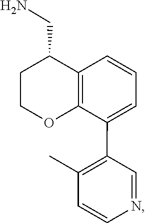 [SMILES: Cc1ccncc1-c1cccc2c1OCC[C@@H]2CN] | 5a |
| 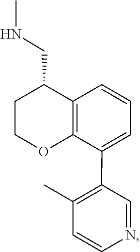 [SMILES: CNC[C@H]1CCOc2c(-c3cnccc3C)cccc21] | 6 |
| 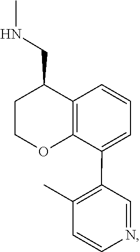 [SMILES: CNC[C@@H]1CCOc2c(-c3cnccc3C)cccc21] | 6a |
| 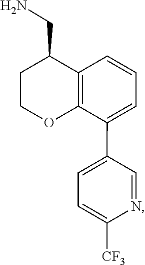 [SMILES: NC[C@@H]1CCOc2c(-c3ccc(C(F)(F)F)nc3)cccc21] | 7 |
| 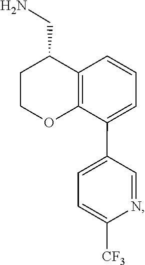 [SMILES: NC[C@H]1CCOc2c(-c3ccc(C(F)(F)F)nc3)cccc21] | 7a |
| 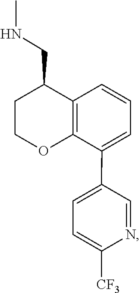 [SMILES: CNC[C@@H]1CCOc2c(-c3ccc(C(F)(F)F)nc3)cccc21] | 8 |
| 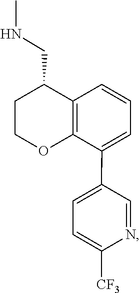 [SMILES: CNC[C@H]1CCOc2c(-c3ccc(C(F)(F)F)nc3)cccc21] | 8a |
| 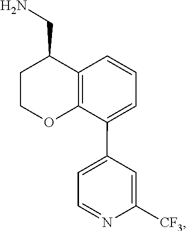 [SMILES: NC[C@@H]1CCOc2c(-c3ccnc(C(F)(F)F)c3)cccc21] | 9 |
| 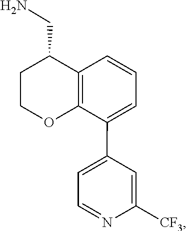 [SMILES: NC[C@H]1CCOc2c(-c3ccnc(C(F)(F)F)c3)cccc21] | 9a |
| 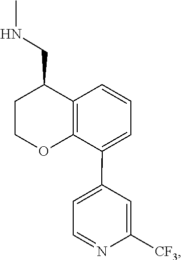 [SMILES: CNC[C@@H]1CCOc2c(-c3ccnc(C(F)(F)F)c3)cccc21] | 10 |
| 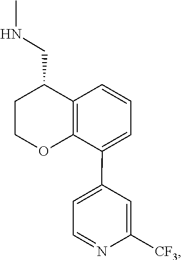 [SMILES: CNC[C@H]1CCOc2c(-c3ccnc(C(F)(F)F)c3)cccc21] | 10a |
| 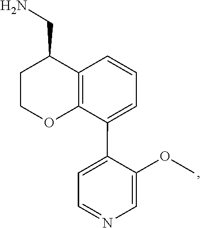 [SMILES: COc1cnccc1-c1cccc2c1OCC[C@H]2CN] | 11 |
| 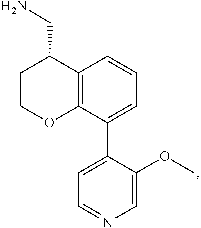 [SMILES: COc1cnccc1-c1cccc2c1OCC[C@@H]2CN] | 11a |
| 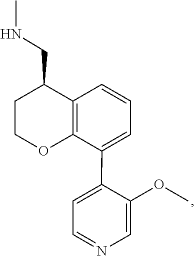 [SMILES: CNC[C@@H]1CCOc2c(-c3ccncc3OC)cccc21] | 12 |
| 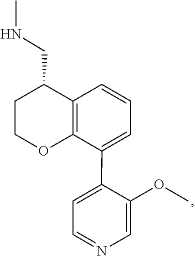 [SMILES: CNC[C@H]1CCOc2c(-c3ccncc3OC)cccc21] | 12a |
| 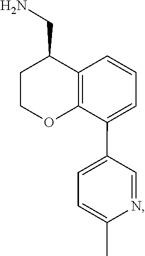 [SMILES: Cc1ccc(-c2cccc3c2OCC[C@H]3CN)cn1] | 13 |
| 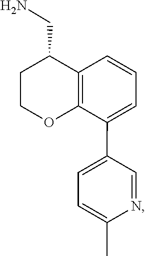 [SMILES: Cc1ccc(-c2cccc3c2OCC[C@@H]3CN)cn1] | 13a |
| 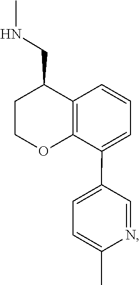 [SMILES: CNC[C@@H]1CCOc2c(-c3ccc(C)nc3)cccc21] | 14 |
| 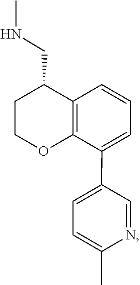 [SMILES: CNC[C@H]1CCOc2c(-c3ccc(C)nc3)cccc21] | 14a |
| 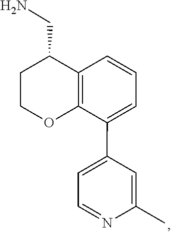 [SMILES: Cc1cc(-c2cccc3c2OCC[C@@H]3CN)ccn1] | 15 |
| 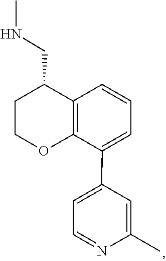 [SMILES: CNC[C@H]1CCOc2c(-c3ccnc(C)c3)cccc21] | 16 |
| 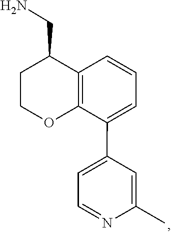 [SMILES: Cc1cc(-c2cccc3c2OCC[C@H]3CN)ccn1] | 17 |
| 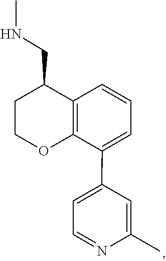 [SMILES: CNC[C@@H]1CCOc2c(-c3ccnc(C)c3)cccc21] | 18 |
| 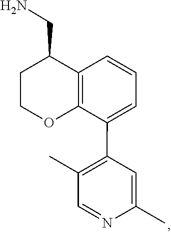 [SMILES: Cc1cc(-c2cccc3c2OCC[C@H]3CN)c(C)cn1] | 19 |
| 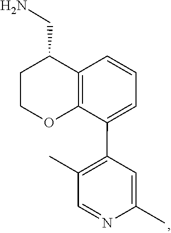 [SMILES: Cc1cc(-c2cccc3c2OCC[C@@H]3CN)c(C)cn1] | 19a |
| 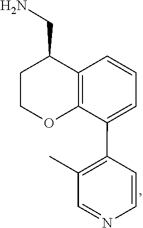 [SMILES: Cc1cnccc1-c1cccc2c1OCC[C@H]2CN] | 20 |
| 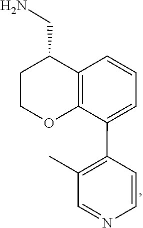 [SMILES: Cc1cnccc1-c1cccc2c1OCC[C@@H]2CN] | 20a |
| 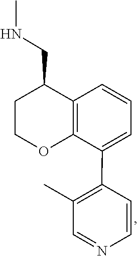 [SMILES: CNC[C@@H]1CCOc2c(-c3ccncc3C)cccc21] | 21 |
| 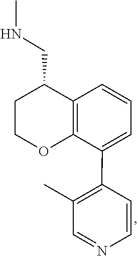 [SMILES: CNC[C@H]1CCOc2c(-c3ccncc3C)cccc21] | 21a |
| 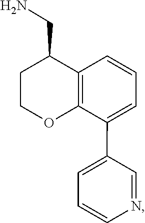 [SMILES: NC[C@@H]1CCOc2c(-c3cccnc3)cccc21] | 22 |
| 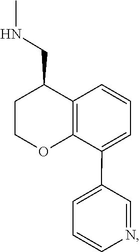 [SMILES: CNC[C@@H]1CCOc2c(-c3cccnc3)cccc21] | 23 |
| 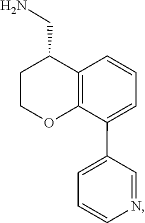 [SMILES: NC[C@H]1CCOc2c(-c3cccnc3)cccc21] | 24 |
| 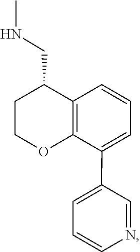 [SMILES: CNC[C@H]1CCOc2c(-c3cccnc3)cccc21] | 25 |
| 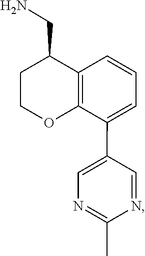 [SMILES: Cc1ncc(-c2cccc3c2OCC[C@H]3CN)cn1] | 26 |
| 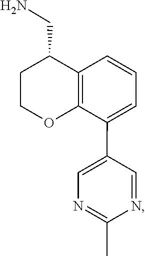 [SMILES: Cc1ncc(-c2cccc3c2OCC[C@@H]3CN)cn1] | 26a |
| 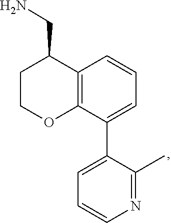 [SMILES: Cc1ncccc1-c1cccc2c1OCC[C@H]2CN] | 27 |
| 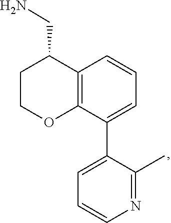 [SMILES: Cc1ncccc1-c1cccc2c1OCC[C@@H]2CN] | 27a |
| 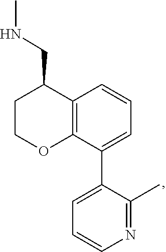 [SMILES: CNC[C@@H]1CCOc2c(-c3cccnc3C)cccc21] | 28 |
| 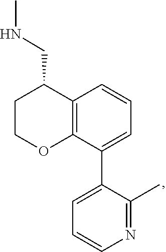 [SMILES: CNC[C@H]1CCOc2c(-c3cccnc3C)cccc21] | 28a |
| 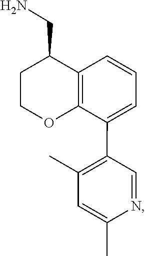 [SMILES: Cc1cc(C)c(-c2cccc3c2OCC[C@H]3CN)cn1] | 29 |
| 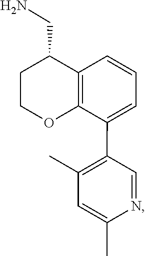 [SMILES: Cc1cc(C)c(-c2cccc3c2OCC[C@@H]3CN)cn1] | 29a |
| 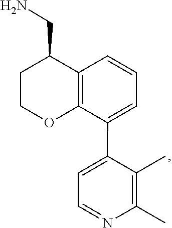 [SMILES: Cc1nccc(-c2cccc3c2OCC[C@H]3CN)c1C] | 30 |
| 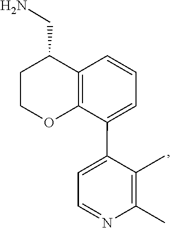 [SMILES: Cc1nccc(-c2cccc3c2OCC[C@@H]3CN)c1C] | 30a |
| 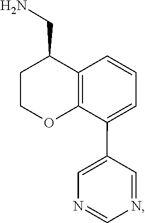 [SMILES: NC[C@@H]1CCOc2c(-c3cncnc3)cccc21] | 31 |
| 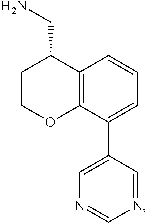 [SMILES: NC[C@H]1CCOc2c(-c3cncnc3)cccc21] | 31a |
| 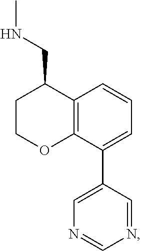 [SMILES: CNC[C@@H]1CCOc2c(-c3cncnc3)cccc21] | 32 |
| 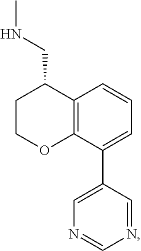 [SMILES: CNC[C@H]1CCOc2c(-c3cncnc3)cccc21] | 32a |
| 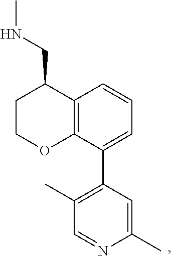 [SMILES: CNC[C@@H]1CCOc2c(-c3cc(C)ncc3C)cccc21] | 33 |
| 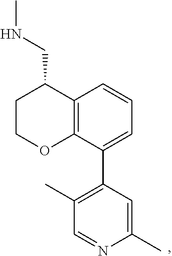 [SMILES: CNC[C@H]1CCOc2c(-c3cc(C)ncc3C)cccc21] | 33a |
| 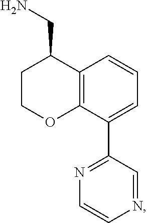 [SMILES: NC[C@@H]1CCOc2c(-c3cnccn3)cccc21] | 34 |
| 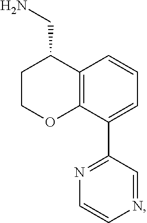 [SMILES: NC[C@H]1CCOc2c(-c3cnccn3)cccc21] | 34a |
| 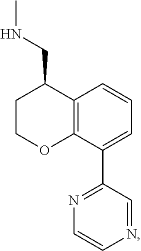 [SMILES: CNC[C@@H]1CCOc2c(-c3cnccn3)cccc21] | 35 |
| 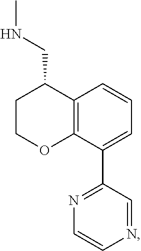 [SMILES: CNC[C@H]1CCOc2c(-c3cnccn3)cccc21] | 35a |
| 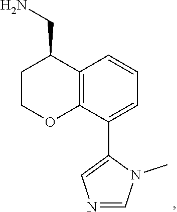 [SMILES: Cn1cncc1-c1cccc2c1OCC[C@H]2CN] | 36 |
| 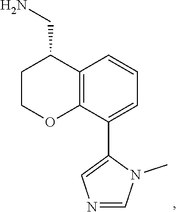 [SMILES: Cn1cncc1-c1cccc2c1OCC[C@@H]2CN] | 36a |
| 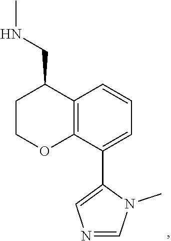 [SMILES: CNC[C@@H]1CCOc2c(-c3cncn3C)cccc21] | 37 |
| 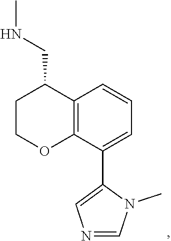 [SMILES: CNC[C@H]1CCOc2c(-c3cncn3C)cccc21] | 37a |
| 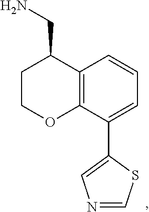 [SMILES: NC[C@@H]1CCOc2c(-c3cncs3)cccc21] | 38 |
| 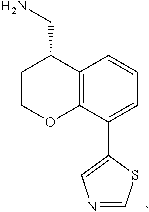 [SMILES: NC[C@H]1CCOc2c(-c3cncs3)cccc21] | 38a |
| 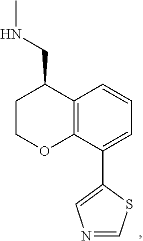 [SMILES: CNC[C@@H]1CCOc2c(-c3cncs3)cccc21] | 39 |
| 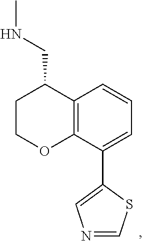 [SMILES: CNC[C@H]1CCOc2c(-c3cncs3)cccc21] | 39a |
| 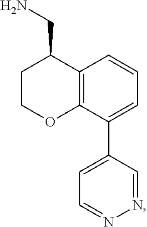 [SMILES: NC[C@@H]1CCOc2c(-c3ccnnc3)cccc21] | 40 |
| 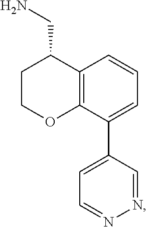 [SMILES: NC[C@H]1CCOc2c(-c3ccnnc3)cccc21] | 40a |
| 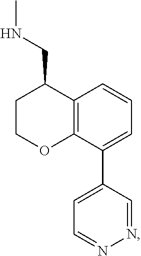 [SMILES: CNC[C@@H]1CCOc2c(-c3ccnnc3)cccc21] | 41 |
| 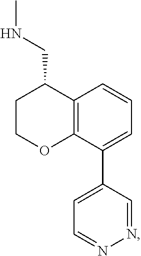 [SMILES: CNC[C@H]1CCOc2c(-c3ccnnc3)cccc21] | 41a |
| 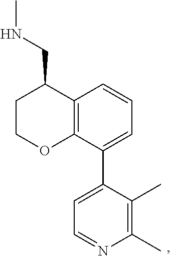 [SMILES: CNC[C@@H]1CCOc2c(-c3ccnc(C)c3C)cccc21] | 42 |
| 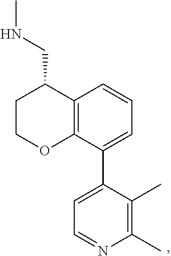 [SMILES: CNC[C@H]1CCOc2c(-c3ccnc(C)c3C)cccc21] | 42a |
| 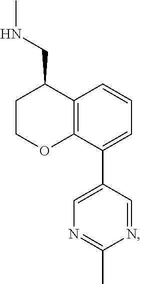 [SMILES: CNC[C@@H]1CCOc2c(-c3cnc(C)nc3)cccc21] | 43 |
| 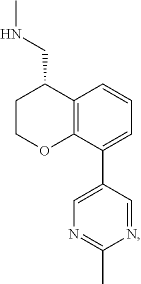 [SMILES: CNC[C@H]1CCOc2c(-c3cnc(C)nc3)cccc21] | 43a |
| 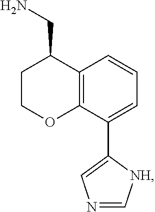 [SMILES: NC[C@@H]1CCOc2c(-c3cnc[nH]3)cccc21] | 44 |
| 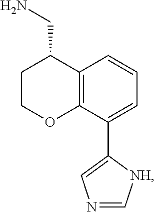 [SMILES: NC[C@H]1CCOc2c(-c3cnc[nH]3)cccc21] | 44a |
| 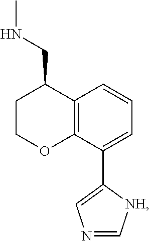 [SMILES: CNC[C@@H]1CCOc2c(-c3cnc[nH]3)cccc21] | 45 |
| 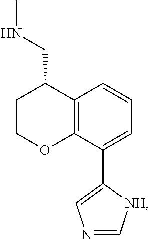 [SMILES: CNC[C@H]1CCOc2c(-c3cnc[nH]3)cccc21] | 45a |
| 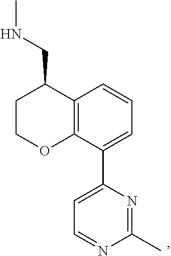 [SMILES: CNC[C@@H]1CCOc2c(-c3ccnc(C)n3)cccc21] | 46 |
| 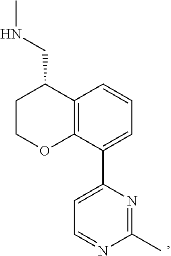 [SMILES: CNC[C@H]1CCOc2c(-c3ccnc(C)n3)cccc21] | 46a |
| 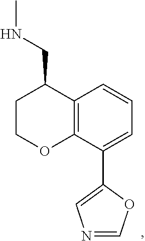 [SMILES: CNC[C@@H]1CCOc2c(-c3cnco3)cccc21] | 47 |
| 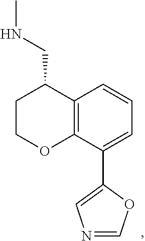 [SMILES: CNC[C@H]1CCOc2c(-c3cnco3)cccc21] | 47a |
| 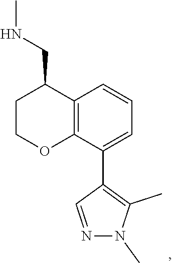 [SMILES: CNC[C@@H]1CCOc2c(-c3cnn(C)c3C)cccc21] | 48 |
| 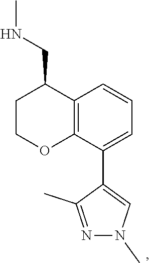 [SMILES: CNC[C@@H]1CCOc2c(-c3cn(C)nc3C)cccc21] | 49 |
| 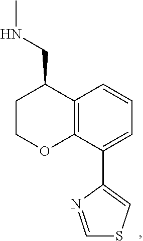 [SMILES: CNC[C@@H]1CCOc2c(-c3cscn3)cccc21] | 50 |
| 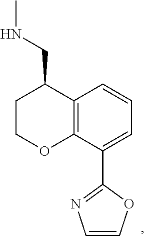 [SMILES: CNC[C@@H]1CCOc2c(-c3ncco3)cccc21] | 51 |
| 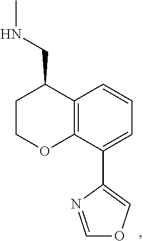 [SMILES: CNC[C@@H]1CCOc2c(-c3cocn3)cccc21] | 52 |
| 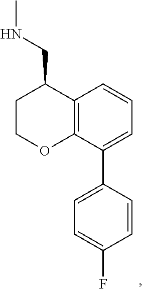 [SMILES: CNC[C@@H]1CCOc2c(-c3ccc(F)cc3)cccc21] | 53 |
| 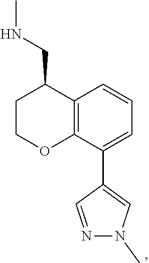 [SMILES: CNC[C@@H]1CCOc2c(-c3cnn(C)c3)cccc21] | 54 |
| 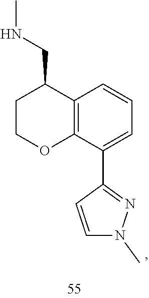 [SMILES: CNC[C@@H]1CCOc2c(-c3ccn(C)n3)cccc21] | 55 |
| 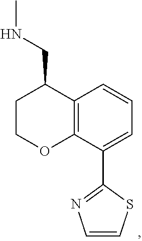 [SMILES: CNC[C@@H]1CCOc2c(-c3nccs3)cccc21] | 56 |
| 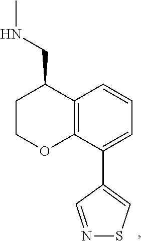 [SMILES: CNC[C@@H]1CCOc2c(-c3cnsc3)cccc21] | 57 |
| 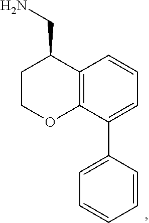 [SMILES: NC[C@@H]1CCOc2c(-c3ccccc3)cccc21] | 58 |
| 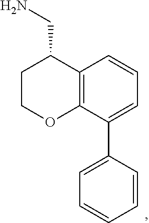 [SMILES: NC[C@H]1CCOc2c(-c3ccccc3)cccc21] | 59 |
| 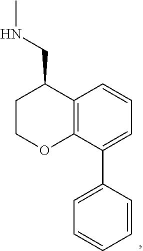 [SMILES: CNC[C@@H]1CCOc2c(-c3ccccc3)cccc21] | 60 |
| 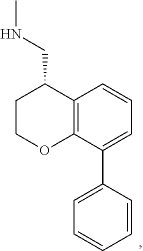 [SMILES: CNC[C@H]1CCOc2c(-c3ccccc3)cccc21] | 61 |
| 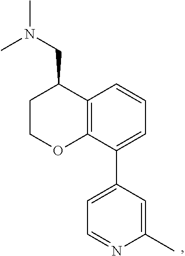 | 62 |
| 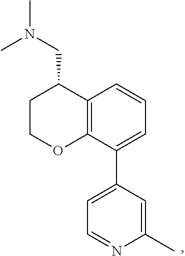 | 63 |
| 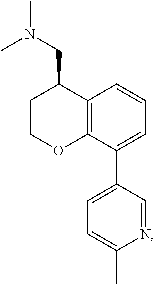 [SMILES: Cc1ccc(-c2cccc3c2OCC[C@H]3CN(C)C)cn1] | 64 |
| 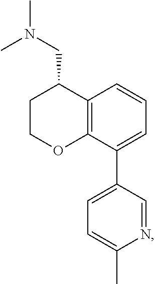 [SMILES: Cc1ccc(-c2cccc3c2OCC[C@@H]3CN(C)C)cn1] | 65 |
| 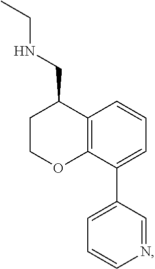 [SMILES: CCNC[C@@H]1CCOc2c(-c3cccnc3)cccc21] | 66 |
| 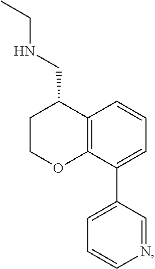 [SMILES: CCNC[C@H]1CCOc2c(-c3cccnc3)cccc21] | 67 |
| 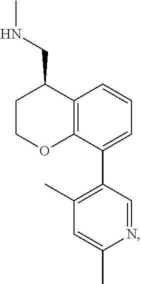 [SMILES: CNC[C@@H]1CCOc2c(-c3cnc(C)cc3C)cccc21] | 68 |
| 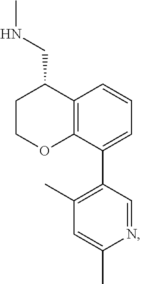 [SMILES: CNC[C@H]1CCOc2c(-c3cnc(C)cc3C)cccc21] | 69 |
| 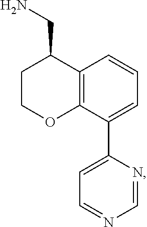 [SMILES: NC[C@@H]1CCOc2c(-c3ccncn3)cccc21] | 70 |
| 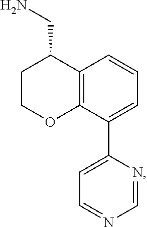 [SMILES: NC[C@H]1CCOc2c(-c3ccncn3)cccc21] | 71 |
| 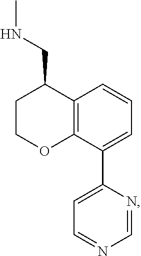 [SMILES: CNC[C@@H]1CCOc2c(-c3ccncn3)cccc21] | 72 |
| 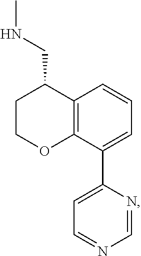 [SMILES: CNC[C@H]1CCOc2c(-c3ccncn3)cccc21] | 73 |
| 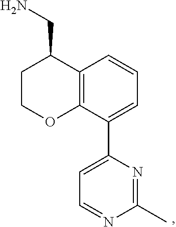 [SMILES: Cc1nccc(-c2cccc3c2OCC[C@H]3CN)n1] | 74 |
| 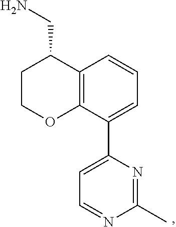 [SMILES: Cc1nccc(-c2cccc3c2OCC[C@@H]3CN)n1] | 75 |
| 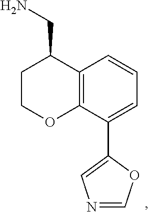 [SMILES: NC[C@@H]1CCOc2c(-c3cnco3)cccc21] | 76 |
| 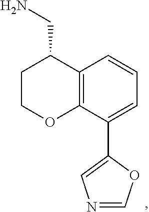 [SMILES: NC[C@H]1CCOc2c(-c3cnco3)cccc21] | 77 |
| 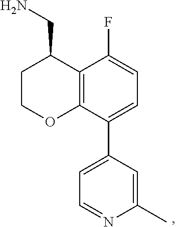 [SMILES: Cc1cc(-c2ccc(F)c3c2OCC[C@H]3CN)ccn1] | 78 |
| 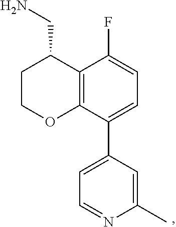 [SMILES: Cc1cc(-c2ccc(F)c3c2OCC[C@@H]3CN)ccn1] | 79 |
| 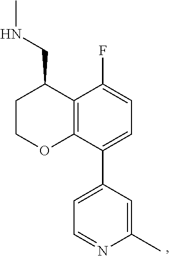 [SMILES: CNC[C@@H]1CCOc2c(-c3ccnc(C)c3)ccc(F)c21] | 80 |
| 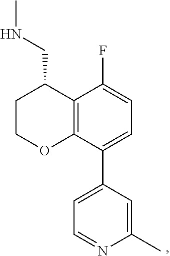 [SMILES: CNC[C@H]1CCOc2c(-c3ccnc(C)c3)ccc(F)c21] | 81 |
| 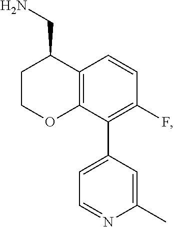 [SMILES: Cc1cc(-c2c(F)ccc3c2OCC[C@H]3CN)ccn1] | 82 |
| 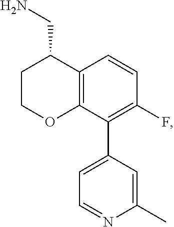 [SMILES: Cc1cc(-c2c(F)ccc3c2OCC[C@@H]3CN)ccn1] | 83 |
| 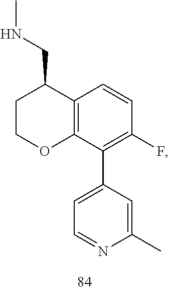 [SMILES: CNC[C@@H]1CCOc2c1ccc(F)c2-c1ccnc(C)c1] | 84 |
| 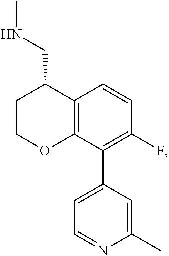 [SMILES: CNC[C@H]1CCOc2c1ccc(F)c2-c1ccnc(C)c1] | 85 |
| 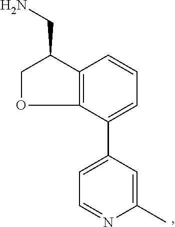 [SMILES: Cc1cc(-c2cccc3c2OC[C@H]3CN)ccn1] | 86 |
| 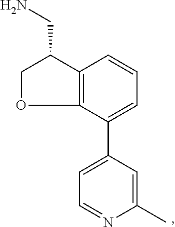 [SMILES: Cc1cc(-c2cccc3c2OC[C@@H]3CN)ccn1] | 87 |
| 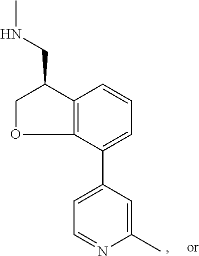 [SMILES: CNC[C@@H]1COc2c(-c3ccnc(C)c3)cccc21] | 88 |
| 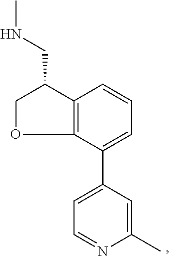 [SMILES: CNC[C@H]1COc2c(-c3ccnc(C)c3)cccc21] | 89 |

or a pharmaceutically acceptable salt of any of the aforementioned compounds.

Methods

The Diagnostic and Statistical Manual of Mental Disorders, Fifth Ed., (the “DSM-5”), published by the American Psychiatric Association in 2013, and as amended or supplemented, provides a standard diagnostic system upon which persons of skill rely for diagnosis of various diseases and disorders, and is hereby incorporated by reference in its entirety. The DSM-5 attempts to capture the large proportion of patients with subsyndromal mixed symptoms with the inclusion of the mixed specifier. Additionally, the International Statistical Classification of Diseases (ICD 10) coding system is a recognized system to communicate about specific diagnoses (e.g., in the United States for billing purposes), and is hereby incorporated by reference in its entirety. For example, Chapter 6 of the ICD 10 is directed to codes for diseases of the nervous system.

The methods of the disclosure relate to the use of compounds and compositions disclosed herein to treat neurological or psychiatric diseases or disorders. In some embodiments, the neurological or psychiatric diseases or disorders is described in the DSM-5, as amended or supplemented, or the International Statistical Classification of Diseases (ICD 10) coding system.

Non-limiting examples of classes of neurological or psychiatric diseases or disorders include Movement Disorders, Cognitive Disorders, Pain, Neurodevelopmental Disorders; Schizophrenia Spectrum and Other Psychotic Disorders; Bipolar and Related Disorders; Depressive Disorders; Anxiety Disorders; Obsessive-Compulsive and Related Disorders; Trauma- and Stressor-Related Disorders; Dissociative Disorders; Somatic Symptom and Related Disorders; Feeding and Eating Disorders; Elimination Disorders; Sleep-Wake Disorders; Sexual Dysfunctions; Gender Dysphoria; Disruptive, Impulse-Control, and Conduct Disorders; Substance-Related and Addictive Disorders; Neurocognitive Disorders; Personality Disorders; Paraphilic Disorders; Other Mental Disorders; and Medication-Induced Movement Disorders and Other Adverse Effects of Medication.

Non-limiting examples of classes of neurological or psychiatric diseases or disorders include:

Movement Disorders

Tremor; Dyskinesia; Dystonia; Tics; Dysphonia; Ataxia (e.g., spinocerebellar ataxia); Myoclonus; Essential Tremor; Epilepsy; Tardive Dyskinesia; Restless Leg Syndrome; Tourette Syndrome; Multiple System Atrophy (MSA); Multiple Sclerosis; Huntington's Disease; Parkinson's Disease; Parkinsonism; Parkinson's disease tremor, Atypical Parkinsonisms (including, for example, Dementia with Lewy Bodies, Progressive Supranuclear Palsy, MSA and Corticobasal Syndrome); Wilson's Disease; Stroke. Examples of akinesias and akinetic-rigid syndromes include Parkinson's disease, drug-induced Parkinsonism, postencephalitic Parkinsonism, secondary Parkinsonism, Parkinson plus syndromes, atypical Parkinsonism, idiopathic Parkinsonism, progressive supranuclear palsy, multiple system atrophy, corticobasal degeneration, Parkinsonism-ALS dementia complex and basal ganglia calcification, medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors. Examples of dyskinesias include drug (e.g., L-DOPA) induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalized myoclonus and focal myoclonus), tics (including simple tics, complex tics and symptomatic tics). Examples of dystonias include generalized dystonia, idiopathic dystonia, drug-induced dystonia, symptomatic dystonia, paroxysmal dystonia, focal dystonia, blepharospasm, oromandibular dystonia, spasmodic dysphonia, spasmodic torticollis, axial dystonia, dystonic writer's cramp and hemiplegic dystonia. Other examples of movement diseases or disorders include stereotypic movement disorder, persistent (chronic) motor disorder, medication-induced movement disorder, psychogenic movement disorders, substance/medication-induced movement disorder, extrapyramidal movement disorders, hyperkinetic movement disorders, hypokinetic movement disorders, alternating hemiplegia, Angelman syndrome, Hallervorden-Spatz Disease, ataxia, dentate cerebellar ataxia, ataxia telangiectasia (Louis-Bar syndrome), Friedreich's Ataxia, hereditary spinal ataxia, hereditary spinal sclerosis, Machado-Joseph Disease, spinocerebellar ataxia, progressive myoclonic ataxia, athetosis, ballismus, blepharospasm (eye twitching), cerebral palsy, tardive dystonia, tardive dyskinesia, idiopathic torsion dystonia, torsion dystonia, focal dystonia, idiopathic familial dystonia, idiopathic nonfamilial dystonia, cervical dystonia (spasmodic torticollis), primary dystonia, orofacial dystonia, developmental coordination disorder, bulbospinal muscular atrophy (Kennedy's Disease), Shy-Drager Syndrome, and Stiff-Person (Stiff-Man) Syndrome. In some embodiments, the present disclosure provides a method of treating one or more symptoms of epilepsy and/or seizures, including abdominal epilepsy, absence seizure, acquired epilepsy, acquired epileptiform aphasia, Aicardi syndrome, Alpers' disease, Alpers-Huttenlocher syndrome, Angelman syndrome, benign focal epilepsy, benign focal epilepsy of childhood, benign intracranial hypertension, benign rolandic epilepsy (BRE), CDKL5 disorder, childhood absence epilepsy, dentate cerebellar ataxia, Doose syndrome, Dravet syndrome, dyscognitive focal seizure, epilepsy with grand mal seizures, epilepsy with myoclonic-absences, epileptic hemiplegia, febrile seizures, focal seizure, frontal lobe epilepsy, generalized tonic-clonic seizures, genetic epilepsy, Glut1 deficiency syndrome, hypothalamic hamartoma, idiopathic epilepsy, idiopathic generalized epilepsy, idiopathic localization-related epilepsies, idiopathic partial epilepsy, idiopathic seizure, juvenile absence epilepsy, juvenile myoclonic epilepsy, Lafora disease, Lafora progressive myoclonus epilepsy, Landau-Kleffner syndrome, Lassueur-Graham-Little syndrome, Lennox syndrome, Lennox-Gastaut syndrome, medically refractory epilepsy, mesial-temporal lobe sclerosis, myoclonic seizure, neonatal epilepsy, occipital lobe epilepsy, Ohtahara syndrome, Panayiotopoulos syndrome, parietal lobe epilepsy, PCDH19 epilepsy, photosensitive epilepsy, progressive myoclonic epilepsies, Rasmussen's encephalitis, Rasmussen's syndrome, refractory epilepsy, seizure disorder, status epilepticus, Sturge-Weber syndrome, symptomatic generalized epilepsy, symptomatic partial epilepsy, TBCK-related ID syndrome, temporal lobe epilepsy, temporal lobe seizures, tonic-clonic seizure, West syndrome, tremor, cerebellar tremor, cerebellar outflow tremor, intention tremor, essential tremor, benign essential tremor, Parkinsonian tremor, and medication-induced postural tremor.

Cognitive Disorders

Alzheimer's disease; Cognitive Impairments; Dementia (including, e.g., Semantic Dementia; Frontotemporal Dementia; Dementia with Depressive Features; Persisting, Subcortical Dementia; Dementia with Lewy Bodies; Parkinsonism-ALS Dementia Complex; Dementia Associated with another disease or disorder, including Alzheimer's Disease; Ischemia; Multi-Infarct Dementia; Trauma; Vascular Problems; Stroke; HIV Disease; Parkinson's Disease; Huntington's Disease; Down Syndrome; Pick's Disease; Creutzfeldt-Jacob Disease; Perinatal Hypoxia, or Substance abuse), Delirium; Amnestic Disorders; or Age Related Cognitive Decline. Cognitive Disorders includes a decline in cognitive functions or cognitive domains, e.g., working memory, attention and vigilance, verbal learning and memory, visual learning and memory, reasoning and problem solving (e.g., executive function, speed of processing and/or social cognition). In particular, cognitive impairment may indicate deficits in attention, disorganized thinking, slow thinking, difficulty in understanding, poor concentration, impairment of problem solving, poor memory, difficulties in expressing thoughts, and/or difficulties in integrating thoughts, feelings and behavior, or difficulties in extinction of irrelevant thoughts. Cognitive Disorders can manifest as a deficit in cognition (cognitive domains as defined by the DSM-5 are: complex attention, executive function, learning and memory, language, perceptual-motor, social cognition); and is sometimes associated with a deficit in dopamine signaling; and is sometimes associated with basal ganglia dysfunction; and is sometimes associated with dysregulated locomotor activity; and is sometimes associated with impairment of prefrontal cortex functioning.

Pain

Fibromyalgia; Neuropathic Pain (including, e.g., post herpetic (or post-shingles) neuralgia, reflex sympathetic dystrophy/causalgia or nerve trauma, phantom limb pain, carpal tunnel syndrome, and peripheral neuropathy (such as diabetic neuropathy or neuropathy arising from chronic alcohol use)), Sensitization Accompanying Neuropathic Pain, Inflammatory Pain; Acute Pain; Nociceptive Pain; Arthritis Pain; Rheumatoid Arthritis; Osteoarthritis; Joint Pain; Musculoskeletal Pain; Back Pain; Dorsalgia; Bulging Disc; Hip Pain; Visceral Pain; Headache; Tension Headache; Acute Tension Headache; Chronic Tension Headache; Chronic Cluster Headache; Common Migraine; Classic Migraine; Cluster Headache; Mixed Headache; Post-Traumatic Headache; Eye Strain Headache; Short-Lasting Unilateral Neuralgiform (SUNCT) Headache; SUNCT Syndrome, Herpes Zoster; Acute Herpes Zoster; Shingles; Postherpetic Neuralgia (Shingles); Causalgia; Central Pain; Central Pain Syndrome; Chronic Back Pain; Neuralgia; Neuropathic Pain Syndrome; Neuropathy; Diabetic Neuropathy; Diabetes-Related Neuropathy; Diabetes-Related Nerve Pain; Fibrositis; Peripheral Neuropathy Caused by Chemotherapy; Peripheral Nerve Disease; Peripheral Neuropathy; Nerve Pain; Nerve Trauma; Sensitization Accompanying Neuropathic Pain; Complex Regional Pain Syndrome; Compression Neuropathy; Craniofacial Pain; Chronic Joint Pain; Chronic Knee Pain; Chronic Pain Syndrome; Cancer Pain; Trigeminal Neuralgia; Tic Doloreaux; Reflex Sympathetic Causalgia; Painful Peripheral Neuropathy; Spinal Nerve Injury; Arachnoiditis; Spinal Pain; Bernhardt-Roth Syndrome (Meralgia Parasthetica); Carpal Tunnel Syndrome; Cerebrospinal Fluid Syndrome; Charcot-Marie-Tooth Disease; Hereditary Motor and Sensory Neuropathy; Peroneal Muscular Atrophy; Cluster-Tic Syndrome; Coccygeal Pain Syndromes; Compartment Syndrome; Degenerative Disc Disease; Failed Back Surgery Syndrome; Genito-Pelvic Pain/Penetration Disorder; Gout; Inflammatory Pain; Lumbar Radiculopathy; Neuroma (Painful Scar); Pain Associated with Multiple Sclerosis; Pelvic Floor Disorders; Phantom Limb Pain; Piriformis Syndrome; Psychogenic Pain; Radicular Pain Syndrome; Raeder's Syndrome; Referred Pain; Reflex Sympathetic Dystrophy Syndrome; Sciatica; Sciatica Pain: Scoliosis; Slipped Disc; Somatic Pain; Spinal Stenosis; Stiff-Person Syndrome/Stiff-Man Syndrome; Stump Pain; Sympathetically Maintained Pain; Tolosa-Hunt Syndrome; Whiplash; Pain Associated with Lyme Disease.

Neurodevelopmental Disorders

Intellectual Disability (Intellectual Developmental Disorder); Global Developmental Delay; Unspecified Intellectual Disability (Intellectual Developmental Disorder); Language Disorder; Speech Sound Disorder; Childhood-Onset Fluency Disorder (Stuttering); Social (Pragmatic) Communication Disorder; Unspecified Communication Disorder; Autism Spectrum Disorder (including, e.g., Asperger's syndrome; Pervasive Developmental Disorder; Rett Syndrome; and Fragile X Syndrome); Attention-Deficit/Hyperactivity Disorder; Other Specified Attention-Deficit/Hyperactivity Disorder; Unspecified Attention-Deficit/Hyperactivity Disorder; Specific Learning Disorder; Childhood Learning Disorder; Developmental Coordination Disorder; Stereotypic Movement Disorder; Tic Disorders; Other Specified Tic Disorder; Unspecified Tic Disorder; Other Specified Neurodevelopmental Disorder; Unspecified Neurodevelopmental Disorder.

Schizophrenia Spectrum and Other Psychotic Disorders

Schizotypal (Personality) Disorder; Delusional Disorder; Brief Psychotic Disorder; Shared Psychotic Disorder Schizophreniform Disorder; Schizophrenia (paranoid, disorganized, catatonic, or undifferentiated); Schizoaffective Disorder; Substance/Medication-Induced Psychotic Disorder; Psychotic Disorder Due to Another Medical Condition; Catatonia Associated With Another Mental Disorder (Catatonia Specifier); Catatonic Disorder Due to Another Medical Condition; Unspecified Catatonia; Other Specified Schizophrenia Spectrum and Other Psychotic Disorder; Unspecified Schizophrenia Spectrum and Other Psychotic Disorder. Schizophrenia is a disorder of unknown origin, which usually appears for the first time in early adulthood and is marked by characteristics such as psychotic symptoms, phasic progression and development, and/or deterioration in social behavior and professional capability. Characteristic psychotic symptoms are disorders of thought content (e.g., multiple, fragmentary, incoherent, implausible or simply delusional contents, or ideas of persecution) and of mentality (e.g., loss of association, flight of imagination, incoherence up to incomprehensibility), as well as disorders of perceptibility (e.g., hallucinations), emotions (e.g., superficial or inadequate emotions), self-perceptions, intentions, impulses, and/or inter-human relationships, and psychomotoric disorders (e.g., catatonia). Other symptoms are also associated with this disorder. Schizophrenia is classified into subgroups: the paranoid type, characterized by delusions and hallucinations and absence of thought disorder, disorganized behavior, and affective flattening; the disorganized type, also named “hebephrenic schizophrenia,” in which thought disorder and flat affect are present together; the catatonic type, in which prominent psychomotor disturbances are evident, and symptoms may include catatonic stupor and waxy flexibility; and the undifferentiated type, in which psychotic symptoms are present but the criteria for paranoid, disorganized, or catatonic types have not been met. The symptoms of schizophrenia normally manifest themselves in three broad categories: positive, negative and cognitive symptoms. Positive symptoms are those which represent an “excess” of normal experiences, such as hallucinations and delusions. Negative symptoms are those where the subject suffers from a lack of normal experiences, such as anhedonia and lack of social interaction. The cognitive symptoms relate to cognitive impairment in schizophrenics, such as lack of sustained attention and deficits in decision making.

Bipolar and Related Disorders

Bipolar I Disorder; Bipolar II Disorder; Cyclothymic Disorder; Substance/Medication-Induced Bipolar and Related Disorder; Bipolar and Related Disorder Due to Another Medical Condition; Other Specified Bipolar and Related Disorder; Unspecified Bipolar and Related Disorder; Specifiers for Bipolar and Related Disorders. Bipolar disorders (including both bipolar I and bipolar II) are serious psychiatric disorders that have a prevalence of approximately 2% of the population, and affects both genders alike. It is a relapsing-remitting condition characterized by cycling between elevated (i.e., manic) and depressed moods, which distinguishes it from other disorders such as major depressive disorder and schizophrenia. Bipolar I is defined by the occurrence of a full manic episode, although most individuals experience significant depression. Symptoms of mania include elevated or irritable mood, hyperactivity, grandiosity, decreased need for sleep, racing thoughts and in some cases, psychosis. The depressive episodes are characterized by anhedonia, sad mood, hopelessness, poor self-esteem, diminished concentration and lethargy. Bipolar II is defined as the occurrence of a major depressive episode and hypomanic (less severe mania) episode although subjects spend considerably more time in the depressive state. Other related conditions include cyclothymic disorder.

Depressive Disorders

Depression, Disruptive Mood Dysregulation Disorder; Major Depressive Disorder (MDD) (Unipolar Depression); Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; Unspecified Depressive Disorder

Anxiety Disorders

Anxiety; Separation Anxiety Disorder; Selective Mutism; Specific Phobia; Social Anxiety Disorder (Social Phobia); Panic Disorder; Panic Attack Specifier; Agoraphobia; Generalized Anxiety Disorder; Substance/Medication-Induced Anxiety Disorder; Anxiety Disorder Due to Another Medical Condition; Other Specified Anxiety Disorder; Unspecified Anxiety Disorder. Anxiety disorders are characterized by fear, worry, and uneasiness, usually generalized and unfocused as an overreaction to a situation. Anxiety disorders differ in the situations or types of objects that induce fear, anxiety, or avoidance behavior, and the associated cognitive ideation. Anxiety differs from fear in that anxiety is an emotional response to a perceived future threat while fear is associated with a perceived or real immediate threat. They also differ in the content of the associated thoughts or beliefs. Examples of anxiety disorders include separation anxiety disorder, selective mutism, specific phobia, social anxiety disorder (social phobia), panic disorder, panic attack specifier, agoraphobia, generalized anxiety disorder, substance/medication-induced anxiety disorder, anxiety disorder due to another medical condition, illness anxiety disorder, social (pragmatic) communication disorder, other specified anxiety disorder, and unspecified anxiety disorder; stressor-related disorders, including reactive attachment disorder, disinhibited social engagement disorder, posttraumatic stress disorder (PTSD), acute stress disorder, and adjustment disorders.

Obsessive-Compulsive and Related Disorders

Obsessive-Compulsive Disorder; Body Dysmorphic Disorder; Hoarding Disorder; Trichotillomania (Hair-Pulling Disorder); Excoriation (Skin-Picking) Disorder; Substance/Medication-Induced Obsessive-Compulsive and Related Disorder; Obsessive-Compulsive and Related Disorder Due to Another Medical Condition; Other Specified Obsessive-Compulsive and Related Disorder; Unspecified Obsessive-Compulsive and Related Disorder

Trauma- and Stressor-Related Disorders

Reactive Attachment Disorder; Disinhibited Social Engagement Disorder; Posttraumatic Stress Disorder; Acute Stress Disorder; Adjustment Disorders; Other Specified Trauma- and Stressor-Related Disorder; Unspecified Trauma- and Stressor-Related Disorder.

Dissociative Disorders

Dissociative Identity Disorder; Dissociative Amnesia; Depersonalization/Derealization Disorder; Other Specified Dissociative Disorder; Unspecified Dissociative Disorder.

Somatic Symptom and Related Disorders

Somatic Symptom Disorder; Illness Anxiety Disorder; Conversion Disorder (Functional Neurological Symptom Disorder); Psychological Factors Affecting Other Medical Conditions; Factitious Disorder; Other Specified Somatic Symptom and Related Disorder; Unspecified Somatic Symptom and Related Disorder.

Feeding and Eating Disorders

Pica; Rumination Disorder; Avoidant/Restrictive Food Intake Disorder; Anorexia Nervosa; Bulimia Nervosa; Binge-Eating Disorder; Other Specified Feeding or Eating Disorder; Unspecified Feeding or Eating Disorder.

Elimination Disorders

Enuresis; Encopresis; Other Specified Elimination Disorder; Unspecified Elimination Disorder.

Sleep-Wake Disorders

Insomnia Disorder; Hypersomnolence Disorder; Narcolepsy; Obstructive Sleep Apnea Hypopnea; Central Sleep Apnea; Sleep-Related Hypoventilation; Circadian Rhythm Sleep-Wake Disorders; Non-Rapid Eye Movement Sleep Arousal Disorders; Nightmare Disorder; Rapid Eye Movement (REM) Sleep Behavior Disorder; Restless Legs Syndrome; Substance/Medication-Induced Sleep Disorder; Other Specified Insomnia Disorder; Unspecified Insomnia Disorder; Other Specified Hypersomnolence Disorder; Unspecified Hypersomnolence Disorder; Other Specified Sleep-Wake Disorder; Unspecified Sleep-Wake Disorder.

Sexual Dysfunctions

Delayed Ejaculation; Erectile Disorder; Female Orgasmic Disorder; Female Sexual Interest/Arousal Disorder; Genito-Pelvic Pain/Penetration Disorder; Male Hypoactive Sexual Desire Disorder; Premature (Early) Ejaculation; Substance/Medication-Induced Sexual Dysfunction; Other Specified Sexual Dysfunction; Unspecified Sexual Dysfunction.

Gender Dysphoria

Gender Dysphoria; Other Specified Gender Dysphoria; Unspecified Gender Dysphoria.

Disruptive, Impulse-Control, and Conduct Disorders

Social Disorder; Oppositional Defiant Disorder; Intermittent Explosive Disorder; Conduct Disorder; Antisocial Personality Disorder; Pyromania; Kleptomania; Other Specified Disruptive, Impulse-Control, and Conduct Disorder; Unspecified Disruptive; Impulse-Control, and Conduct Disorder.

Substance-Related and Addictive Disorders

Addiction; Alcohol Use Disorder; Alcohol Intoxication; Alcohol Withdrawal; Unspecified Alcohol-Related Disorder; Fetal Alcohol Syndrome; Caffeine Intoxication; Caffeine Withdrawal; Unspecified Caffeine-Related Disorder; Cannabis Use Disorder; Cannabis Intoxication; Cannabis Withdrawal; Unspecified Cannabis-Related Disorder; Phencyclidine Use Disorder; Other Hallucinogen Use Disorder; Phencyclidine Intoxication; Other Hallucinogen Intoxication; Hallucinogen Persisting Perception Disorder; Unspecified Phencyclidine-Related Disorder; Unspecified Hallucinogen-Related Disorder; Inhalant Use Disorder; Inhalant Intoxication; Unspecified Inhalant-Related Disorder; Opioid Use Disorder; Opioid Intoxication; Opioid Withdrawal; Unspecified Opioid-Related Disorder; Sedative, Hypnotic, or Anxiolytic Use Disorder; Sedative, Hypnotic, or Anxiolytic Intoxication; Sedative, Hypnotic, or Anxiolytic Withdrawal; Unspecified Sedative-, Hypnotic-, or Anxiolytic-Related Disorder; Stimulant Use Disorder; Stimulant Intoxication; Stimulant Withdrawal; Unspecified Stimulant-Related Disorder; Tobacco Use Disorder; Tobacco Withdrawal; Unspecified Tobacco-Related Disorder; Other (or Unknown) Substance Use Disorder; Other (or Unknown) Substance Intoxication; Other (or Unknown) Substance Withdrawal; Unspecified Other (or Unknown) Substance-Related Disorder; Gambling Disorder.

Neurocognitive Disorders

Delirium; Other Specified Delirium; Unspecified Delirium; Major and Mild Neurocognitive Disorders; Major or Mild Neurocognitive Disorder Due to Alzheimer's Disease; Major or Mild Frontotemporal Neurocognitive Disorder; Major or Mild Neurocognitive Disorder With Lewy Bodies; Major or Mild Vascular Neurocognitive Disorder; Major or Mild Neurocognitive Disorder Due to Traumatic Brain Injury; Substance/Medication-Induced Major or Mild Neurocognitive Disorder; Major or Mild Neurocognitive Disorder Due to HIV Infection; Major or Mild Neurocognitive Disorder Due to Prion Disease; Major or Mild Neurocognitive Disorder Due to Parkinson's Disease; Major or Mild Neurocognitive Disorder Due to Huntington's Disease; Major or Mild Neurocognitive Disorder Due to Another Medical Condition; Major or Mild Neurocognitive Disorder Due to Multiple Etiologies; Unspecified Neurocognitive Disorder.

Personality Disorders

Dimensional Models for Personality Disorders; General Personality Disorder; Paranoid Personality Disorder; Schizoid Personality Disorder; Schizotypal Personality Disorder; Antisocial Personality Disorder; Borderline Personality Disorder; Histrionic Personality Disorder; Narcissistic Personality Disorder; Avoidant Personality Disorder; Dependent Personality Disorder; Obsessive-Compulsive Personality Disorder; Personality Change Due to Another Medical Condition; Other Specified Personality Disorder; Unspecified Personality Disorder.

Paraphilic Disorders

Voyeuristic Disorder; Exhibitionistic Disorder; Frotteuristic Disorder; Sexual Masochism Disorder; Sexual Sadism Disorder; Pedophilic Disorder; Fetishistic Disorder; Transvestic Disorder; Other Specified Paraphilic Disorder; Unspecified Paraphilic Disorder.

Other Mental Disorders

Other Specified Mental Disorder Due to Another Medical Condition; Unspecified Mental Disorder Due to Another Medical Condition; Other Specified Mental Disorder; Unspecified Mental Disorder.

Medication-Induced Movement Disorders and Other Adverse Effects of Medication

Neuroleptic-Induced Parkinsonism Other Medication-Induced Parkinsonism; Neuroleptic Malignant Syndrome; Medication-Induced Acute Dystonia; Medication-Induced Acute Akathisia; Tardive Dyskinesia; Tardive Dystonia Tardive Akathisia; Medication-Induced Postural Tremor; Other Medication-Induced Movement Disorder; Antidepressant Discontinuation Syndrome; Other Adverse Effect of Medication.

Symptoms of Neurological or Psychiatric Diseases and Disorders

Neurological or psychiatric diseases or disorders can manifest as a variety of symptoms. Non-limiting examples of symptoms of neurological or psychiatric diseases or disorders include symptoms such as apathy, depression, anxiety, cognitive impairment, psychosis, aggression, agitation, impulse control disorders, sleep disorders, elevated or irritable mood, hyperactivity, grandiosity, decreased need for sleep, racing thoughts and in some cases, psychosis, anhedonia, sad mood, hopelessness, poor self-esteem, diminished concentration and lethargy, amyotrophic lateral sclerosis, primary lateral sclerosis, progressive muscular atrophy, progressive bulbar (atrophy) palsy, pseudobulbar palsy spinal muscular atrophy diseases (e.g., SMA type I, also called Werdnig-Hoffmann disease, SMA type II, SMA type III, also called Kugelberg-Welander disease, and Kennedy Disease, also called progressive spinobulbar muscular atrophy), Hallervorden-Spatz disease, Seitelberger disease (Infantile Neuroaxonal Dystrophy), adrenoleukodystrophy, Alexander Disease, autosomal dominant cerebellar ataxia (ADCA), pure autonomic failure (Bradbury-Eggleston Syndrome), CADASIL Syndrome, and neuronal ceroids lipofuscinose disorders such as Batten Disease (Spielmeyer-Vogt-Sjögren)), senile dementia, Early Onset Alzheimer's Disease, Alzheimer's type dementia, cognition, memory loss, amnesia/amnestic syndrome, disturbances of consciousness, coma, lowering of attention, speech disorder, agnosia, aphasia, apraxia, Mild Cognitive Impairment (MCI), benign forgetfulness, mild neurocognitive disorder, major neurocognitive disorder, neurocognitive disorder due to disease (e.g., Huntington's Disease, Parkinson's disease, Prion Disease, Traumatic Brain Injury, HIV or AIDS), Binswanger's Disease (subcortical leukoencephalopathy), and Capgras Syndrome; or any other symptoms associated with a neurological or psychiatric disease or disorder disclosed herein.

Pharmaceutical Compositions

In certain embodiments, provided herein is a composition (e.g., a pharmaceutical composition) comprising a compound described herein and a pharmaceutically acceptable excipient or carrier. In some embodiments, provided herein is a method of treating neurological or psychiatric diseases and disorders in a subject in need thereof in a subject, comprising administering an effective amount of a compound or a pharmaceutical composition described herein. Examples of carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present disclosure or pharmaceutical composition thereof) or aid in the manufacturing of the pharmaceutical product (i.e., medicament).

Compositions of the present disclosure may be administered orally, parenterally, by inhalation, topically, rectally, nasally, buccally, sublingually, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. Pharmaceutically acceptable compositions of this disclosure may be orally administered in any orally acceptable dosage form including capsules, tablets, aqueous suspensions or solutions.

The amount of compounds of the present disclosure that may be combined with the carrier materials to produce a composition in a single dosage form will vary depending upon a variety of factors, including the host treated and the particular mode of administration. It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

The compounds and compositions of the disclosure are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds and compositions of the present disclosure will be decided by the attending physician within the scope of sound medical judgment.

Combination Therapy

In some embodiments, compounds disclosed herein provide a method of treating a neurological and/or psychiatric disease or disorder described herein, comprising administering a compound disclosed herein in conjunction with one or more pharmaceutical agents. Suitable pharmaceutical agents that may be used in combination with the compounds of the present invention include anti-Parkinson's drugs, anti-Alzheimer's drugs, anti-depressants, anti-psychotics, anti-ischemics, CNS depressants, anti-cholinergics, nootropics, epilepsy medication, attention (e.g., ADD/ADHD) medications, sleep-promoting medications, wakefulness-promoting medications, and pain medications.

Suitable anti-Parkinson's drugs include dopamine replacement therapy (e.g. L-DOPA, carbidopa, COMT inhibitors such as entacapone or tolcapone), dopamine agonists (e.g. D1 agonists, D2 agonists, mixed D1/D2 agonists, bromocriptine, pergolide, cabergoline, ropinirole, pramipexole, piribedil, or apomorphine in combination with domperidone), histamine H2 antagonists, monoamine oxidase inhibitors (such as selegiline, rasagiline, safinamideand tranylcypromine), certain atypical antipsychotics such as pimavanserin (a non-dopaminergic atypical antipsychotic and inverse agonist of the serotonin 5-HT_{2A} receptor), and amantadine.

In some embodiments, compounds of the invention can be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone or tolcapone, MAO A/B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include beta-secretase inhibitors, gamma-secretase inhibitors, cholinesterase inhibitors such as donepezil, galantamine or rivastigmine, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists.

Specific suitable anti-depressant and anti-anxiety agents include amitriptyline, clomipramine, doxepin, imipramine and trimipramine; amoxapine, desipramine, citalopram, escitalopram, maprotiline, nortriptyline and protriptyline; fluoxetine, fluvoxamine, paroxetine and sertraline; isocarboxazid, phenelzine, tranylcypromine and selegiline; moclobemide: venlafaxine; desvenlafaxine, duloxetine; aprepitant; bupropion, vilazodone, mirtazapine, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, reboxetine, vortioxetine, clorazepate, and ketamine and pharmaceutically acceptable salts thereof. In some embodiments, suitable anti-depressant and anti-anxiety agents are tianeptine, or pharmaceutically acceptable salts thereof.

Suitable anti-psychotic and mood stabilizer agents include D2 antagonists, 5HT2A antagonists, atypical antipsychotics, lithium, and anticonvulsants.

Specific suitable anti-psychotic and mood stabilizer agents include chlorpromazine, fluphenazine, haloperidol, amisulpride, perphenazine, thioridazine, trifluoperazine, aripiprazole, asenapine, clozapine, olanzapine, paliperidone, brexpiprazole, paliperidone, cariprazine, pimavanserin, illoperidone, lumateperone, MIN-101, quetiapine, risperidone, ziprasidone, lurasidone, flupentixol, levomepromazine, pericyazine, perphenazine, pimozide, prochlorperazine, zuclopenthixol, olanzapine and fluoxetine, lithium, carbamazepine, lamotrigine, valproic acid, iloperidone, thiothixene, gabapentin, tiagabine and pharmaceutically acceptable salts thereof.

Suitable epilepsy medications include levetiracetam, oxcarbazepine, clobazam, retigabine, zonisamide, felbamate, esclicarbazepine acetate, lacosamide, carbamazepine, tiagabine, methsuximide, progabide, valproic acid, lamotrigine, brivaracetam, rufinamide, topiramate and perampanel.

Suitable attention medications include methyl phenidate, atomoxetine, guanfacine, D-amphetamine, lisdexamphetamine, methylamphetamine, and clonidine.

Suitable sleep-promoting medications include ramelteon, triazolam, zopiclone, eszopiclone, zolpidem, temazepam, and trazodone.

Suitable wakefulness-promoting medications include Modafinil, D-Amphetamine, caffeine, and armodafinil.

Suitable pain medications include dextromethorphan, tapentadol, buprenorphine, codeine, fentanyl, hydrocodone, hydromorphone, morphine, naloxegol, oxycodone, tramadol, gabapentil, difluprednate, pregabalin, acetyl salicyclic acid, bromfenac, diclofenac, diflunisal, indomethacin, ketorolac, meoxican, and naproxen.

In some embodiments, compounds and compositions disclosed herein may be used in combination with other therapies. Suitable therapies include psychotherapy, cognitive behavioral therapy, electroconvulsive therapy, transcranial magnetic stimulation, vagus nerve stimulation, and deep-brain stimulation.

### EXAMPLES

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon, as appreciated by those skilled in the art. Preferred methods include, but are not limited to, those described below. The reactions are performed in a solvent or solvent mixture appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure. Examples are depicted with relative stereochemistry except where specifically stated otherwise.

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd} ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

The following abbreviations used hereinbelow have the corresponding meanings:

| | | | |
|---|---|---|---|
| ACN | acetonitrile; | Ac₂O | acetic anhydride; |
| Aq | aqueous; | BSA | bovine serum albumin; |
| BINAP | 2,2′-Bis(diphenylphosphino)- | | |
| | 1,1′-binaphthyl; | | |
| Boc | tert-butyloxycarbonyl; | C | Celsius; |
| CH₂Cl₂ | dichloromethane; | Cs₂CO₃ | cesium carbonate; |
| d | doublet; | dd | doublet of doublets; |
| DCE | 1,2-dichloroethane; | DCM | dichloromethane; |
| DIPEA/DIEA | N,N-diisopropylethylamine; | DMF | N,N-dimethylformamide; |
| DMSO | dimethylsulfoxide; | | |
| EDC | 1-ethyl-3-(3- | | |
| | dimethylaminopropyl)carbodiimide; | | |
| EtOAc | ethyl acetate; | EtOH | ethanol; |
| g | gram; | h | hour(s); |
| HATU | 2-(1H-7-azabenzotriazol-1-yl)-1,1,3,3- | | |
| | tetramethyluronium hexafluorophosphate; | | |
| HOAt | 1-hydroxy-7-azabenzotriazole; | | |
| HPLC | high pressure liquid chromatography; | | |
| IBX | 2-iodoxybenzoic acid; | kg | kilogram; |
| L | liter; | LC | liquid chromatography; |
| LCMS | liquid chromatography and mass | | |
| | spectrometry; | | |
| LiOH | Lithium hydroxide; | MeOH | methanol; |
| MS | mass spectrometry; | M | molar; |
| m | multiplet; | min | minutes; |
| mL | milliliter(s); | μM | micromolar; |
| m/z | mass to charge ratio; | nm | nanometer; |
| nM | nanomolar; | N | normal; |
| NMP | N-methylpyrrolidone; | NMR | nuclear magnetic resonance; |
| Pd(OAc)₂ | palladium(II) acetate; | PS | polymer-supported; |
| PG | protecting group; | pTsOH | p-toluenesulfonic acid; |
| rac | racemic; | rt | room temperature; |
| s | singlet; | sat. | saturated; |
| t | triplet; | TEA | triethylamine; |
| TFA | trifluoroacetic acid; | TFE | trifluoroethanol; |
| THF | tetrahydrofuran; | TLC | thin layer chromatography. |

### Example 1. Preparation of (R)-(8-(pyridin-4-yl)chroman-4-yl)methanamine (Compound 1), (S)-(8-(pyridin-4-yl)chroman-4-yl)methanamine (Compound 2), (R)—N-methyl-1-(8-(pyridin-4-yl)chroman-4-yl)methanamine (Compound 3), and (S)—N-methyl-1-(8-(pyridin-4-yl)chroman-4-yl)methanamine (Compound 4)

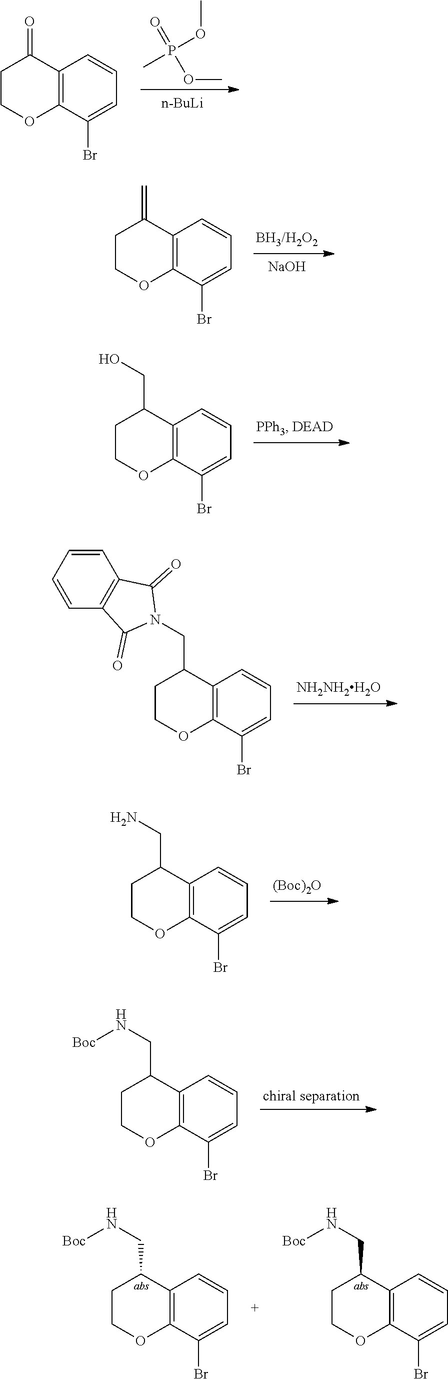

Synthesis of 8-bromo-4-methylenechromane: To a solution of dimethyl methylphosphonate (88.7 g, 715 mmol) in tetrahydrofuran (4 L) was added n-BuLi (2.5 M in tetrahydrofuran, 251.6 mL, 629 mmol) at −60° C. The mixture was stirred at this temperature for 30 minutes and 8-bromochroman-4-one (65 g, 286 mmol) in tetrahydrofuran (300 mL) was added slowly. The mixture was stirred at −50° C. for 2 h, quenched with sat. ammonia chloride solution (300 mL) and extracted with ethyl acetate (550 mL). The organic layer was dried and concentrated in vacuum. The residue was purified by silica gel chromatography using petroleum ether/ethyl acetate (1/1) and then dichloromethane/methanol (10/1) as eluents to give the crude intermediate, which was dissolved in N,N-dimethylformamide (500 mL). To the solution was added water (30 mL), and potassium carbonate (300.3 g, 2200 mmol). The mixture was stirred at 110° C. for 12 h, quenched with water (800 mL), and extracted with ethyl acetate (500 mL×2). The organic layer was dried and concentrated under in vacuum to give a residue, which was again purified by silica gel chromatography using petroleum ether/ethyl acetate (50/1) as an eluent to give 8-bromo-4-methylenechromane (45 g, 51.2%) as a yellow oil. MS(ESI): m/z 225,227 [M+H]⁺.

Synthesis of (8-bromochroman-4-yl)methanol: To a solution of 8-bromo-4-methylenechromane (45 g, 199 mmol) in tetrahydrofuran (100 mL) was added 1 M BH₃·tetrahydrofuran (398 mL, 398 mmol). The mixture was stirred at room temperature for 2 h and sodium hydroxide solution (aq. 2 M, 300 mL) was added followed by water (30% aq. 30 mL). The mixture was stirred at room temperature for 1 h. Ethyl acetate (600 mL) was added to the reaction vessel and the resulting biphasic mixture was separated. The organic phase was triturated with water (200 mL) and brine (200 mL). The combined organic layer was dried and concentrated in vacuum. The resulting oil was purified by flash column chromatography with a gradient elution of petroleum ether/ethyl acetate (100%: 0%) to petroleum ether/ethyl acetate (65%: 35%) to provide (8-bromochroman-4-yl)methanol (40 g, 82.3%) as a colorless oil. MS(ESI): m/z 243, 245 [M+H]⁺.

Synthesis of 2-((8-bromochroman-4-yl)methyl)isoindoline-1,3-dione: To a solution of (8-bromochroman-4-yl)methanol (40 g, 165 mmol) in tetrahydrofuran (600 mL) was added triphenylphosphane (51.9 g, 198 mmol) and diethyl (E)-diazene-1,2-dicarboxylate (34.1 g, 198 mmol) at 0° C. The reaction was stirred at ambient temperature for 1 h and quenched with water (100 mL). The mixture was extracted with ethyl acetate (600 mL×2). The organic layer was dried and concentrated. The crude solid was triturated with methanol (50 mL) to provide 2-((8-bromochroman-4-yl)methyl)isoindoline-1,3-dione (46 g, 74.9%) as a white solid. MS(ESI): m/z 372,374[M+H]⁺.

Synthesis of (8-bromochroman-4-yl)methanamine: To a solution of 2-((8-bromochroman-4-yl)methyl)isoindoline-1,3-dione (46 g, 124 mmol in ethanol (100 mL) was added hydrazine hydrate (37 mL, 740 mmol). The mixture was stirred at 90° C. for 3 h and cooled to room temperature. The solid was filtered off and the filtrate was concentrated to provide (8-bromochroman-4-yl)methanamine (27 g, 90.3%) as a colorless oil. MS(ESI): m/z 242,244 [M+H]⁺.

Synthesis of tert-butyl ((8-bromochroman-4-yl)methyl)carbamate: To a solution of 8(8-bromochroman-4-yl)methanamine (27 g, 112 mmol) in dichloromethane (500 mL) and sodium carbonate solution (500 mL) was added di-tert-butyl dicarbonate (29.3 g, 134 mmol). The reaction was stirred at ambient temperature for 1 h. The separated organic layer was concentrated and purified by silica gel chromatography (petroleum ether/ethyl acetate=2/1) to give tert-butyl ((8-bromochroman-4-yl)methyl)(methyl)carbamate (35 g, 91.4%) as a colorless oil. MS(ESI): m/z 365,367 [M+Na]⁺.

Synthesis of tert-butyl (S)-((8-bromochroman-4-yl)methyl)carbamate (P1) and tert-butyl (R)-((8-bromochroman-4-yl)methyl)carbamate (P2): The racemic tert-butyl ((8-bromochroman-4-yl)methyl)(methyl)carbamate was purified by chiral separation using Instrument: SFC-200 (Thar, Waters); Column: AY 20*250 mm, 10 um (Daicel); Column temperature: 35° C.; Mobile phase: CO2/MEOH (0.2% Methanol Ammonia)=85/15; Flow rate: 100 g/min; Back pressure: 100 bar; Detection wavelength: 214 nm; Cycle time: 3.5 min; Sample solution: 35000 mg dissolved in 600 ml Methanol; Injection volume: 2 ml to get the desired isomer. MS (ESI): m/z 365, 367 [M+Na]⁺. tert-butyl (S)-((8-bromochroman-4-yl)methyl)carbamate (P1): purity: 100%, 214 nm; ee %: 100%, 16.0 g yellow solid obtained, retention time: 2.83 min, yield: 45.7%. tert-butyl (R)-((8-bromochroman-4-yl)methyl)carbamate (P2): purity: 100%, 214 nm; ee %: 100%, 16.0 g yellow solid obtained, retention time: 3.56 min, yield: 45.7%.

### Synthesis of tert-butyl (R)-((8-(4,4,5,5-tetramethyl-1,3,2-dioxaborolan-2-yl)chroman-4-yl)methyl)carbamate

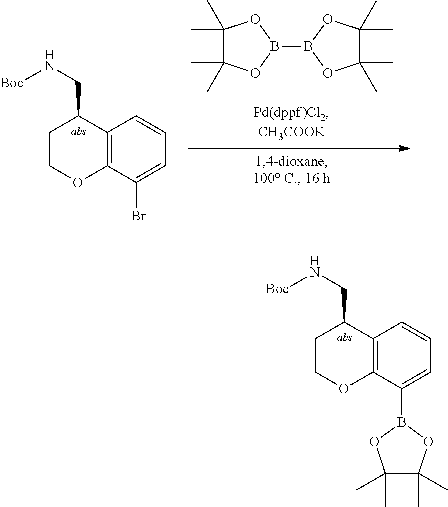

To a solution of tert-butyl N-{[(4R)-8-bromo-3,4-dihydro-2H-1-benzopyran-4-yl]methyl}carbamate (10.0 g, 29.2 mmol, 1.0 eq) and 4,4,5,5-tetramethyl-2-(4,4,5,5-tetramethyl-1,3,2-dioxaborolan-2-yl)-1,3,2-dioxaborolane (8.88 g, 35.0 mmol, 1.2 eq) in 1,4-dioxane (300 mL) was added 1,1′-Bis(diphenylphosphino)ferrocene-palladium(II)dichloride (Pd(dppf)Cl₂) dichloromethane complex (1.19 g, 1.46 mmol, 0.05 eq) and potassium acetate (8.59 g, 87.6 mmol, 3.0 eq) then the mixture is stirred at 100° C. for overnight. Diluted the mixture with ethyl acetate (200 mL) and the solid was filtered off. The filtration was concentrated, and then purified by silica gel column (petrol ether/ethyl acetate=10/1) to give tert-butyl N-{[(4R)-8-(4,4,5,5-tetramethyl-1,3,2-dioxaborolan-2-yl)-3,4-dihydro-2H-1-benzopyran-4-yl]methyl}carbamate (6.2 g, yield 54.8%). (ESI): m/z: 412.1[M+Na]⁺.

### Synthesis of tert-butyl (R)-((8-bromochroman-4-yl)methyl)(methyl)carbamate

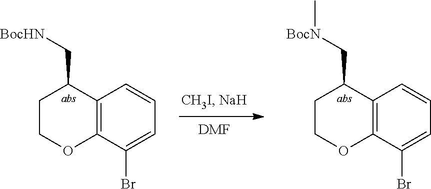

To a solution of tert-butyl (R)-((8-bromochroman-4-yl)methyl)carbamate (1 g, 2.92 mmol) in N,N-dimethylformamide (20 mL) was added NaH (350 mg, 8.76 mmol) at 0° C. After stirred at 0° C. for 30 mins, iodomethane (828 mg, 5.84 mmol) was added. The reaction was stirred at ambient temperature for 2 h. Upon the completion, ethyl acetate (30 mL) and water (50 mL) were added and the mixture was triturated with brine (2×80 mL), dried and concentrated. The resulting oil was purified by flash column chromatography with a gradient elution of petroleum ether (100%) to petroleum ether (90%) and ethyl acetate (10%) to provide tert-butyl (R)-((8-bromochroman-4-yl)methyl)(methyl)carbamate (897 mg, yield: 86%) as a colorless oil. ESI: m/z=256,258[M−100+H]⁺.

### Synthesis of tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate

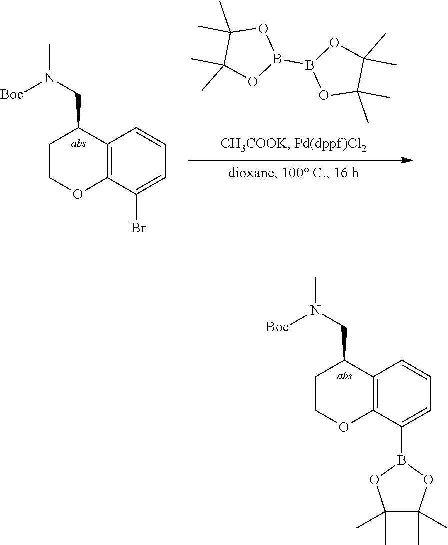

To a solution of tert-butyl N-{[(4R)-8-bromo-3,4-dihydro-2H-1-benzopyran-4-yl]methyl}-N-methylcarbamate (4.5 g, 12.6 mmol, 1.0 eq) and 4,4,5,5-tetramethyl-2-(4,4,5,5-tetramethyl-1,3,2-dioxaborolan-2-yl)-1,3,2-dioxaborolane (4.79 g, 18.9 mmol, 1.5 eq) in 1,4-dioxane (150 mL) was added 1,1′-Bis(diphenylphosphino)ferrocene-palladium(II)dichloride dichloromethane complex (514 mg, 0.630 mmol, 0.05 eq) and potassium acetate (3.70 g, 37.8 mmol, 3.0 eq) then the mixture was stirred at 100° C. for overnight. Water (100 mL) and ethyl acetate (100 mL) was added to the reaction vessel and the resulting biphasic mixture was transferred to a separatory funnel. The layers were separated and the organic phase was triturated with saturated aqueous NaCl (2×150 mL). The combined organics were dried over anhydrous Na₂SO₄, filtered and concentrated in vacuo to afford crude product. The crude product was purified by silica gel column (petrol ether/ethyl acetate=5/1) to get the desired product tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate (5.1 g, yield 100%, purity 95%). MS(ESI): m/z 426 [M+Na]⁺.

### Synthesis of tert-butyl (R)-((8-(pyridin-4-yl)chroman-4-yl)methyl)carbamate

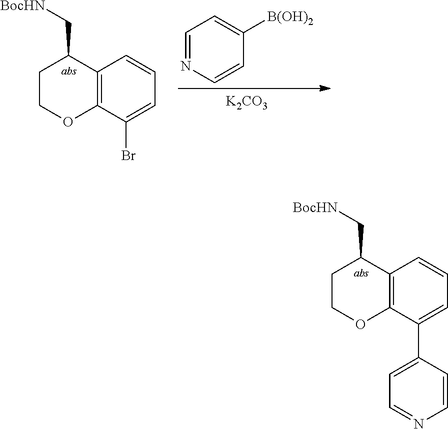

A mixture of tert-butyl (R)-((8-bromochroman-4-yl)methyl)carbamate (3.42 g, 10 mmol), pyridin-4-ylboronic acid (15 mmol, 1.83 g), potassium carbonate (4.14 g, 30 mmol) and Pd(PPh₃)₄ (578 mg, 0.5 mmol) in dioxane/water (30/10 mL) was heated to 100 degrees and stirred overnight. The mixture was diluted with water (50 mL) and then extracted with ethyl acetate (100 mL). The separated organic layer was triturated with brine (50 ml), dried over sodium sulfate, filtered and then concentrated in vacuum to get the crude product which was purified by chromatography with petrol ether/ethyl acetate=1/1 to give tert-butyl (R)-((8-(pyridin-4-yl)chroman-4-yl)methyl)carbamate as a pale yellow solid. (2.8 g, yield: 82%). MS(ESI): m/z 341 [M+H]⁺.

### Synthesis of (R)-(8-(pyridin-4-yl)chroman-4-yl)methanamine dihydrochloride salt (Compound 1)

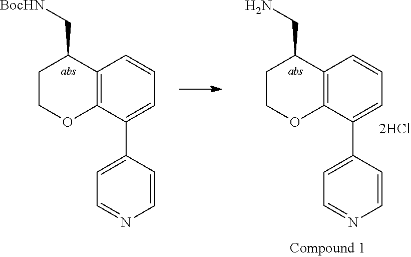 [SMILES: CC(C)(C)OC(=O)NC[C@@H]1CCOc2c(-c3ccncc3)cccc21 ; NC[C@@H]1CCOc2c(-c3ccncc3)cccc21]

A mixture of tert-butyl (R)-((8-(pyridin-4-yl)chroman-4-yl)methyl)carbamate (102 mg, 0.3 mmol) in HCl/ethyl acetate (3N, 10 mL) was stirred at room temperature overnight. The mixture was concentrated in vacuum and then the residue was triturated with ethyl acetate (10 mL) to give (R)-(8-(pyridin-4-yl)chroman-4-yl)methanamine dihydrochloride salt (Compound 1) as a white solid. (60 mg, yield: 63%) MS(ESI): m/z 241 [M+H]⁺. ¹H NMR (400 MHz, DMSO-d₆) δ: 8.93-8.90 (m, 2H), 8.47 (br, 3H), 8.19-8.17 (m, 2H), 7.53-7.51 (m, 1H), 7.46 (dd, J=7.6, 1.6 Hz, 1H), 7.11 (t, J=7.6 Hz, 1H), 4.26-4.24 (m, 2H), 3.34-3.30 (m, 1H), 3.22-3.16 (m, 1H), 3.10-3.05 (m, 1H), 2.21-2.03 (m, 2H).

### Synthesis of tert-butyl (S)-((8-(pyridin-4-yl)chroman-4-yl)methyl)carbamate

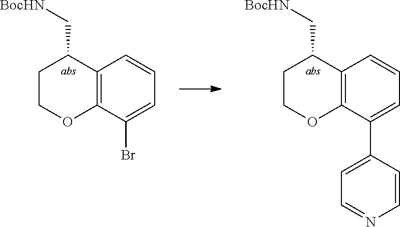 [SMILES: CC(C)(C)OC(=O)NC[C@H]1CCOc2c(Br)cccc21 ; CC(C)(C)OC(=O)NC[C@H]1CCOc2c(-c3ccncc3)cccc21]

A mixture of tert-butyl (S)-((8-bromochroman-4-yl)methyl)carbamate (3.42 g, 10 mmol), pyridin-4-ylboronic acid (15 mmol, 1.83 g), potassium carbonate (4.14 g, 30 mmol) and Pd(PPh₃)₄ (578 mg, 0.5 mmol) in dioxane/water (30/10 mL) was heated to 100 degrees and stirred overnight. The mixture was diluted with water (50 mL) and then extracted with ethyl acetate (100 mL). The separated organic layer was washed with brine (50 ml), dried over sodium sulfate, filtered and then concentrated in vacuum. The crude product was purified by chromatography with petrol ether/ethyl acetate=1/1 to give tert-butyl (S)-((8-(pyridin-4-yl)chroman-4-yl)methyl)carbamate as a pale yellow solid. (2.7 g, yield: 79%). MS(ESI): m/z 341 [M+H]⁺.

### Synthesis of (S)-(8-(pyridin-4-yl)chroman-4-yl)methanamine dihydrochloride salt (Compound 2)

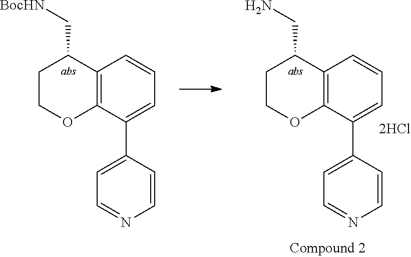 [SMILES: CC(C)(C)OC(=O)NC[C@H]1CCOc2c(-c3ccncc3)cccc21 ; NC[C@H]1CCOc2c(-c3ccncc3)cccc21]

A mixture of tert-butyl (S)-((8-(pyridin-4-yl)chroman-4-yl)methyl)carbamate (102 mg, 0.3 mmol) in HCl/ethyl acetate (3N, 10 mL) was stirred at room temperature overnight. The mixture was concentrated in vacuum and then the residue was triturated with ethyl acetate (10 mL) to give (R)-(8-(pyridin-4-yl)chroman-4-yl)methanamine dihydrochloride salt (Compound 2) as a white solid (60 mg, yield: 63%). MS (ESI): m/z 241 [M+H]⁺. MS (ESI): m/z 241 [M+H]⁺. ¹H NMR (400 MHz, DMSO-d₆) δ: 8.91-8.90 (m, 2H), 8.47 (br, 3H), 8.18 (d, J=6.8 Hz, 2H), 7.53-7.51 (m, 1H), 7.46 (dd, J=7.6, 1.6 Hz, 1H), 7.10 (t, J=7.6 Hz, 1H), 4.26-4.24 (m, 2H), 3.34-3.30 (m, 1H), 3.20-3.16 (m, 1H), 3.10-3.05 (m, 1H), 2.21-2.03 (m, 2H).

### Synthesis of (R)-tert-butyl methyl((8-(pyridin-4-yl)chroman-4-yl)methyl)carbamate

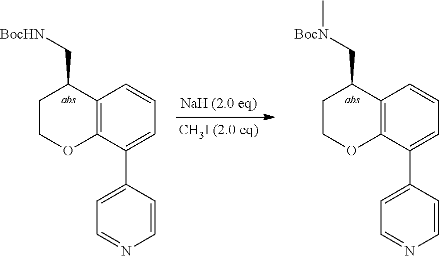 [SMILES: CC(C)(C)OC(=O)NC[C@@H]1CCOc2c(-c3ccncc3)cccc21 ; "CH3I (2.0 eq)" ; CN(C[C@@H]1CCOc2c(-c3ccncc3)cccc21)C(=O)OC(C)(C)C]

To a solution of (R)-tert-butyl ((8-(pyridin-4-yl)chroman-4-yl)methyl)carbamate (200 mg, 587 μmol) in N,N-dimethylformamide (5 mL) was added sodium hydride (28.0 mg, 1.17 mmol) at rt. After stirring at rt for 10 min, iodomethane (166 mg, 1.17 mmol) was added to the mixture and stirred at rt for 30 min. The reaction was quenched with water (20 mL) and the mixture was extracted with ethyl acetate (2×20 mL). The combined organics were dried over anhydrous Na₂SO₄, filtered and concentrated in vacuum. The resulting oil was purified by reverse phase HPLC with a gradient elution of water (100%) and acetonitrile (0%) to water (50%) and acetonitrile (50%) to provide (R)-tert-butylmethyl((8-(pyridin-4-yl)chroman-4-yl)methyl)carbamate (180 mg, yield: 87%) as a yellow solid. MS (ESI): m/z 355 [M+H]⁺.

### Synthesis of (R)—N-methyl-1-(8-(pyridin-4-yl)chroman-4-yl)methanamine dihydrochloride salt (Compound 3)

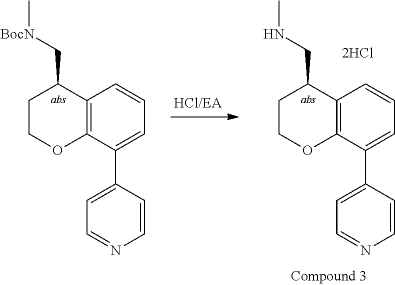 [SMILES: CN(C[C@@H]1CCOc2c(-c3ccncc3)cccc21)C(=O)OC(C)(C)C ; CNC[C@@H]1CCOc2c(-c3ccncc3)cccc21]

To a solution of (R)-tert-butyl methyl((8-(pyridin-4-yl)chroman-4-yl)methyl)carbamate (180 mg, 507 μmol) in ethyl acetate (5 mL) was added HCl/1,4-dioxane (5 mL) at rt. Then the mixture was stirred at rt for 5 h. The mixture was filtered and the precipitate was collected and dried in vacuo to give (R)—N-methyl-1-(8-(pyridin-4-yl)chroman-4-yl)methanamine dihydrochloride salt (Compound 3) (120 mg, yield: 82%) as a yellow solid. MS (ESI): m/z 255 [M+H]⁺. ¹H NMR (400 MHz, CD₃OD) δ: 8.79 (d, J=6.8 Hz, 2H), 8.29 (d, J=6.8 Hz, 2H), 7.52-7.49 (m, 2H), 7.14 (t, J=7.6 Hz, 1H), 4.35-4.29 (m, 2H), 3.43-3.32 (m, 3H), 2.79 (s, 3H), 2.26-2.20 (m, 1H), 2.11-2.06 (m, 1H).

### Synthesis of (S)-tert-butyl methyl((8-(pyridin-4-yl)chroman-4-yl)methyl)carbamate

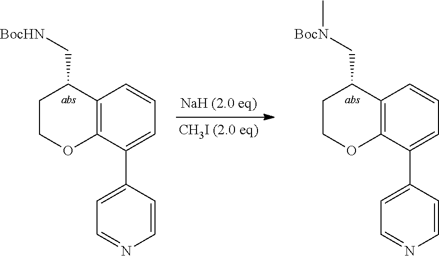 [SMILES: CC(C)(C)OC(=O)NC[C@H]1CCOc2c(-c3ccncc3)cccc21 ; "CH3I (2.0 eq)" ; CN(C[C@H]1CCOc2c(-c3ccncc3)cccc21)C(=O)OC(C)(C)C]

To a solution of (S)-tert-butyl ((8-(pyridin-4-yl)chroman-4-yl)methyl)carbamate (230 mg, 675 μmol) in N,N-dimethylformamide (5 mL) was added sodium hydride (32.1 mg, 1.34 mmol) at rt. After stirring at rt for 10 min, iodomethane (190 mg, 1.34 mmol) was added to the mixture and stirred at rt for 30 min. The reaction was quenched with water (20 mL) and the mixture was extracted with ethyl acetate (2×20 mL). The combined organics were dried over anhydrous Na₂SO₄, filtered and concentrated in vacuo. The resulting oil was purified by reverse phase HPLC with a gradient elution of water (100%) and acetonitrile (0%) to water (50%) and acetonitrile (50%) to provide (S)-tert-butylmethyl((8-(pyridin-4-yl)chroman-4-yl)methyl)carbamate (150 mg, yield: 63%) as a yellow solid. MS (ESI): m/z 355 [M+H]⁺.

### Synthesis of (S)—N-methyl-1-(8-(pyridin-4-yl)chroman-4-yl)methanamine dihydrochloride salt (Compound 4)

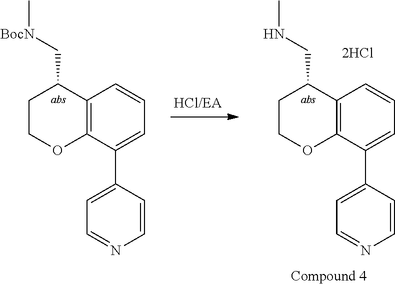 [SMILES: CN(C[C@H]1CCOc2c(-c3ccncc3)cccc21)C(=O)OC(C)(C)C ; CNC[C@H]1CCOc2c(-c3ccncc3)cccc21]

To a solution of (S)-tert-butyl methyl((8-(pyridin-4-yl)chroman-4-yl)methyl)carbamate (150 mg, 423 μmol) in ethyl acetate (5 mL) was added HCl/1,4-dioxane (5 mL) at rt. Then the mixture was stirred at rt for 5 h. The mixture was filtered and the precipitate was collected and dried in vacuum to give (S)—N-methyl-1-(8-(pyridin-4-yl)chroman-4-yl)methanamine dihydrochloride salt (Compound 4) (80 mg, yield: 65%) as a yellow solid. MS (ESI): m/z 255 [M+H]⁺. ¹H NMR (400 MHz, CD₃OD) δ: 8.80 (d, J=6.8 Hz, 2H), 8.30 (d, J=6.8 Hz, 2H), 7.53-7.51 (m, 2H), 7.15 (t, J=7.6 Hz, 1H), 4.36-4.30 (m, 2H), 3.44-3.33 (m, 3H), 2.80 (s, 3H), 2.25-2.21 (m, 1H), 2.13-2.08 (m, 1H).

### Example 2: Synthesis of Compound 5 ((R)-(8-(4-methylpyridin-3-yl)chroman-4-yl)methanamine) and Compound 6

### Synthesis of tert-butyl (R)-((8-(4-methylpyridin-3-yl)chroman-4-yl)methyl)carbamate

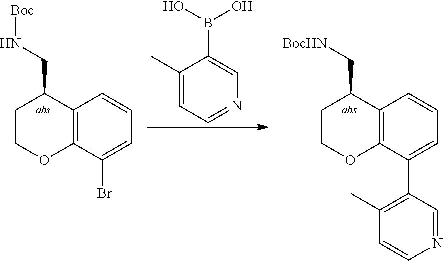

To a solution of tert-butyl (R)-((8-bromochroman-4-yl)methyl)carbamate (382 mg, 1.02 mmol, 1.0 eq) and (4-methylpyridin-3-yl)boronic acid (153 mg, 1.12 mmol, 1.1 eq) in 1,4-dioxane (10 mL)/water (2 ml) was added 1,1′-Bis(diphenylphosphino)ferrocene-palladium(II)(41.7 mg, 0.051 mmol, 0.05 eq) and sodium carbonate (324 mg, 3.06 mmol, 3.0 eq) then the mixture is stirred at 100° C. for 10 h. The mixture was concentrated in vacuum and then purified by silica gel column (dichloromethane/methanol=30/1) to give the target product (300 mg, yield: 83%). MS (ESI): m/z 355 [M+H]⁺.

### Synthesis of Compound 5

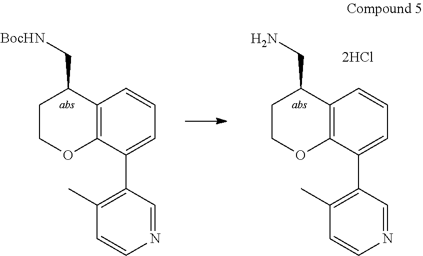 [SMILES: Cc1ccncc1-c1cccc2c1OCC[C@H]2CNC(=O)OC(C)(C)C ; Cc1ccncc1-c1cccc2c1OCC[C@H]2CN]

To a solution of tert-butyl (R)-((8-(4-methylpyridin-3-yl)chroman-4-yl)methyl)carbamate (80 mg, 0.2257 mmol, 1.0 eq) in dichloromethane (5 mL) was added HCl/ethyl acetate (10 ml, 3 N) then the mixture was stirred at room temperature overnight. The mixture was concentrated in vacuum to give the white solid Compound 5 ((R)-(8-(4-methylpyridin-3-yl)chroman-4-yl)methanamine) (47 mg, purity 100%, yield: 63.5%). MS (ESI): m/z 255 [M+H]⁺. ¹H-NMR (500 MHz, CD₃OD) δ 8.73-8.65 (m, 2H), 8.05 (d, J=6 Hz, 1H), 7.49 (d, J=8 Hz, 1H), 7.24-7.13 (m, 2H), 4.25-4.23 (m, 2H), 3.46-3.34 (m, 3H), 2.51 (s, 3H), 2.24-2.05 (m, 2H).

### Synthesis of Compound 6

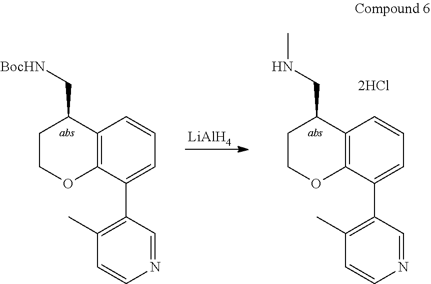

To a solution of tert-butyl (R)-((8-(4-methylpyridin-3-yl)chroman-4-yl)methyl)carbamate (100 mg, 0.282 mmol, 1.0 eq) in tetrahydrofuran (20 mL) was added LiAlH₄ (3 mg, 0.846 mmol, 3.0 eq) then the mixture was reflux for 2 h. The mixture was concentrated in vacuum and then purified by pre-HPLC to give (R)—N-methyl-1-(8-(4-methylpyridin-3-yl)chroman-4-yl)methanamine. The mixture was treated with HCl/ethyl acetate (5 mL, 3N) to give (R)—N-methyl-1-(8-(4-methylpyridin-3-yl)chroman-4-yl)methanaminedihydrochloride salt. (50 mg, purity 99%, yield 52%). MS (ESI): m/z 269 [M+H]⁺. ¹H-NMR (500 MHz, CD₃OD) δ 8.73-8.65 (m, 2H), 8.05 (d, J=6 Hz, 1H), 7.52 (d, J=6.8 Hz, 1H), 7.24-7.13 (m, 2H), 4.25-4.23 (m, 2H), 3.50-3.34 (m, 3H), 2.84 (s, 3H), 2.51 (s, 3H), 2.26-2.09 (m, 2H).

### Example 3: Synthesis of Compound 7, Compound 8

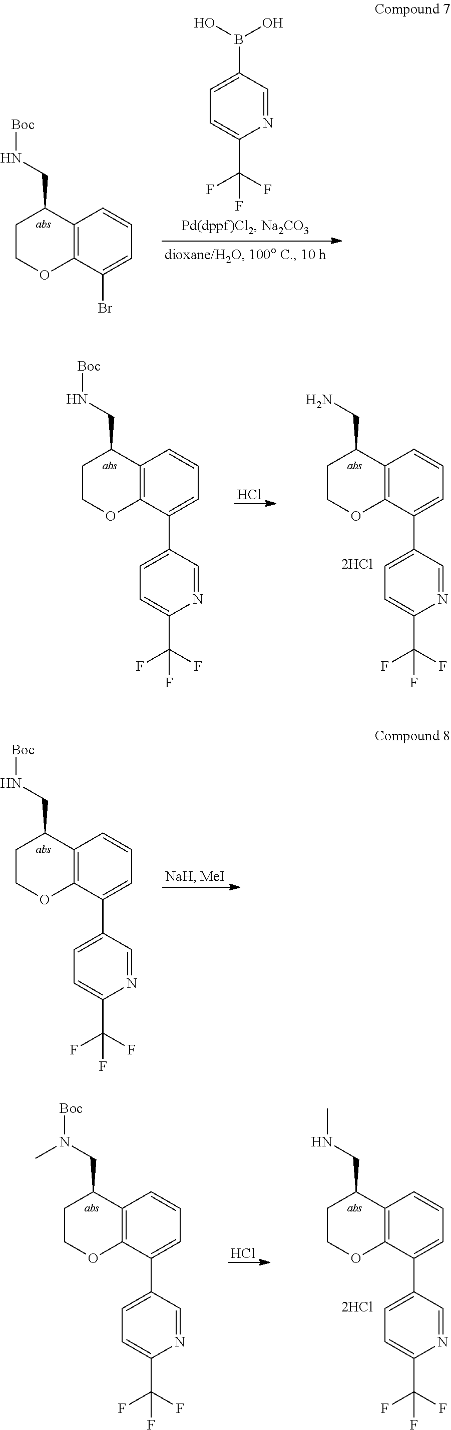

Synthesis of tert-butyl N-{[(4R)-8-(3-methylpyridin-4-yl)-3,4-dihydro-2H-1-benzopyran-4-yl]methyl}carbamate: To a solution of tert-butyl N-{[(4R)-8-bromo-3,4-dihydro-2H-1-benzopyran-4-yl]methyl}-N-methylcarbamate (0.3 g, 0.876 mmol) in dioxane (10 mL) was added Pd(dppf)Cl₂ (64.2 mg, 0.0876 mmol), sodium carbonate (277 mg, 2.62 mmol) and [6-(trifluoromethyl)pyridin-3-yl]boronic acid (215 mg, 1.13 mmol) The reaction mixture was heated to 100° C. and stirred at that temperature for 24 h under nitrogen atmosphere. Ethyl acetate (30 mL) was added to the reaction vessel, and the resulting biphasic mixture was transferred to a separatory funnel. The layers were separated and the organic phase was triturated with brine (4×30 mL). The combined organics were dried over sodium sulfate, filtered and concentrated in vacuum. The resulting solid was purified by flash column chromatography with a gradient elution of petroleum ether (100%) to petroleum ether (70%) and ethyl acetate (30%) to provide tert-butyl (R)-((8-(6-(trifluoromethyl)pyridin-3-yl)chroman-4-yl)methyl)carbamate (180 mg, yield: 37.2%) as a yellow oil. MS (ESI): m/z 409 [M+H]⁺.

Synthesis of 1-[(4R)-8-[6-(trifluoromethyl)pyridin-3-yl]-3,4-dihydro-2H-1-benzopyran-4-yl]methanamine (Compound 7): To a solution of tert-butyl (R)-((8-(6-(trifluoromethyl)pyridin-3-yl)chroman-4-yl)methyl)carbamate (100 mg, 0.244 mmol) was added HCl/ethyl acetate (10 ml) The reaction was stirred at room temperature for 2 h. Upon the completion, the mixture was evaporated in vacuo to dryness, and then freeze-dried to give Compound 7 (57.6 mg yield: 76.5%) as white solid. MS (ESI): m/z 309 [M+H]⁺. ¹H NMR (400 MHz, CD₃OD) δ 8.82 (s, 1H), 8.14 (d, J=8.1 Hz, 1H), 7.84 (d, J=8.1 Hz, 1H), 7.48-7.14 (m, 2H), 7.08 (t, J=7.7 Hz, 1H), 4.48-3.97 (m, 2H), 3.43-3.21 (m, 3H), 2.49-0.83 (m, 2H).

Synthesis of tert-butyl N-methyl-N-{[(4R)-8-[6-(trifluoromethyl)pyridin-3-yl]-3,4-dihydro-2H-1-benzopyran-4-yl]methyl}carbamate: To a solution of tert-butyl N-{[(4R)-8-[6-(trifluoromethyl)pyridin-3-yl]-3,4-dihydro-2H-1-benzopyran-4-yl]methyl}carbamate (120 mg, 0.293 mmol) in N,N-dimethylformamide (10 mL) was added iodomethane (83.1 mg, 0.586 mmol) and sodium hydride (23.4 mg, 0.586 mmol). The reaction was stirred at 0 degree for 2 hours. Water (1.5 mL) was added to the reaction vessel and the resulting biphasic mixture was transferred to a separatory funnel. Ethyl acetate (30 mL) was added to the reaction vessel and the resulting biphasic mixture was transferred to a separatory funnel. The layers were separated and the organic phase was washed with brine (4×30 mL). The combined organics were dried over anhydrous Na₂SO₄, filtered and concentrated in vacuo. The resulting solid was purified by flash column chromatography with a gradient elution of petroleum ether (100%) to petroleum ether (70%) and ethyl acetate (30%) to provide tert-butyl N-methyl-N-{[(4R)-8-[6-(trifluoromethyl)pyridin-3-yl]-3,4-dihydro-2H-1-benzopyran-4-yl]methyl}carbamate (78.3 mg, yield: 63.6%) as a yellow oil. MS (ESI): m/z 423 [M+H]⁺.

Synthesis of methyl({[(4R)-8-[6-(trifluoromethyl)pyridin-3-yl]-3,4-dihydro-2H-1-benzopyran-4-yl]methyl})amine (Compound 8): To a solution of methyl({[(4R)-8-[6-(trifluoromethyl)pyridin-3-yl]-3,4-dihydro-2H-1-benzopyran-4-yl]methyl})amine (80 mg, 0.189 mmol) was added HCl/ethyl acetate (10 ml) The reaction was stirred at room temperature for 2 h. Upon the completion, the mixture was evaporated in vacuo to dryness and then freeze-dried to yield Compound 8 (56.6 mg yield: 92.9%) as white solid. MS (ESI): m/z 323 [M+H]⁺. ¹H NMR (400 MHz, CD₃OD) δ 8.81 (d, J=1.4 Hz, 1H), 8.14 (dd, J=8.1, 1.6 Hz, 1H), 7.84 (d, J=8.1 Hz, 1H), 7.62-7.23 (m, 2H), 7.08 (t, J=7.6 Hz, 1H), 4.48-4.00 (m, 2H), 3.02-2.65 (m, 3H), 2.83 (s, 3H), 2.16 (m, 2H).

### Example 4: Synthesis of Compound 9, Compound 10

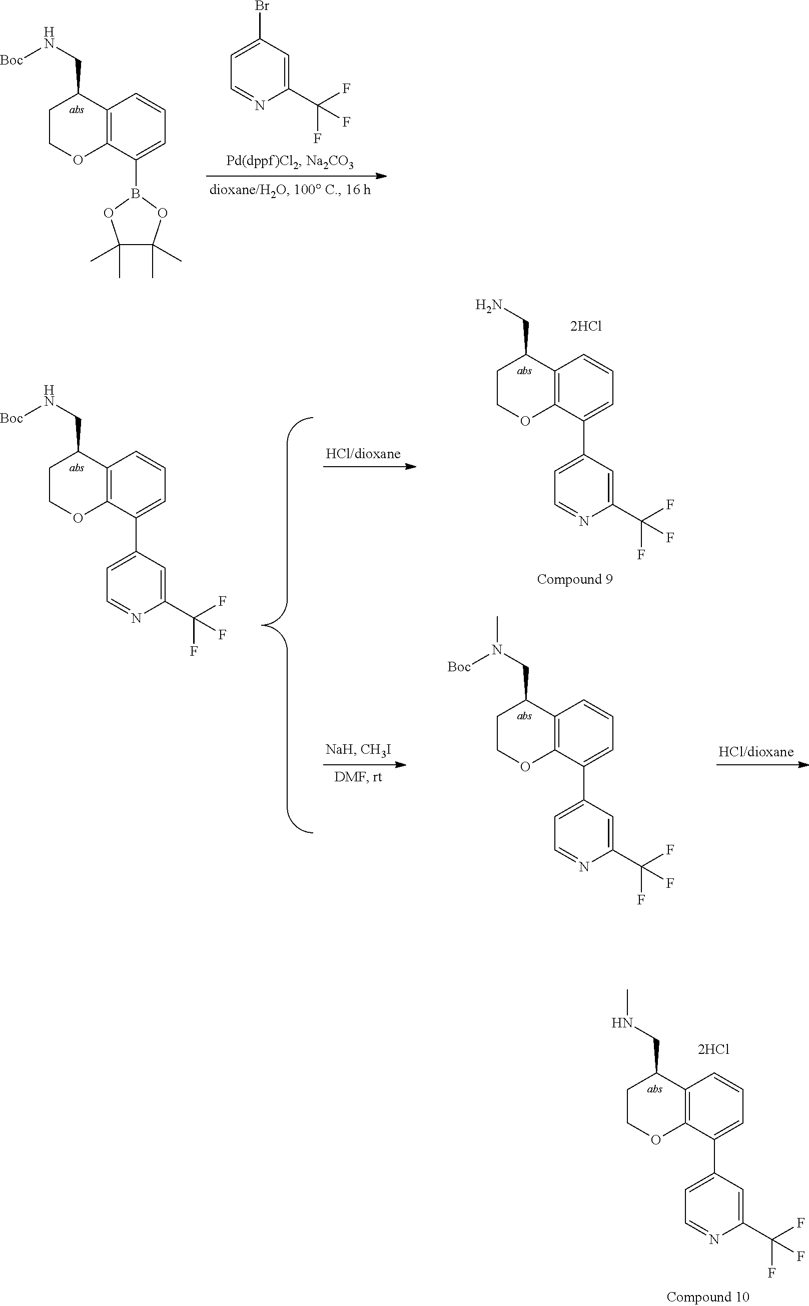 [SMILES: CC(C)(C)OC(=O)NC[C@@H]1CCOc2c(B3OC(C)(C)C(C)(C)O3)cccc21 ; FC(F)(F)c1cc(Br)ccn1 ; CC(C)(C)OC(=O)NC[C@@H]1CCOc2c(-c3ccnc(C(F)(F)F)c3)cccc21 ; CN(C[C@@H]1CCOc2c(-c3ccnc(C(F)(F)F)c3)cccc21)C(=O)OC(C)(C)C ; NC[C@@H]1CCOc2c(-c3ccnc(C(F)(F)F)c3)cccc21 ; CNC[C@@H]1CCOc2c(-c3ccnc(C(F)(F)F)c3)cccc21]

Synthesis of tert-butyl (R)-((8-(2-(trifluoromethyl)pyridin-4-yl)chroman-4-yl)methyl)carbamate: To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (400 mg, 1.02 mmol, 1.0 eq) and 4-bromo-2-(trifluoromethyl)pyridine (253 mg, 1.12 mmol, 1.1 eq) in 1,4-dioxane (10 mL)/water (2 ml) was added 1,1′-Bis(diphenylphosphino)ferrocene-palladium(II) (41.7 mg, 0.051 mmol, 0.05 eq) and sodium carbonate (324 mg, 3.06 mmol, 3.0 eq) then the mixture was stirred at 100° C. for 16 h. The mixture was concentrated, and then the residue was purified by silica gel column (petrol ether/ethyl acetate=3/1) to give the product (390 mg, 93.7% yield). MS (ESI): m/z 409 [M+H]⁺.

Synthesis of Compound 9: To a solution of tert-butyl N-{[(4R)-8-[2-(trifluoromethyl)pyridin-4-yl]-3,4-dihydro-2H-1-benzopyran-4-yl]methyl}carbamate (100 mg, 0.244 mmol, 1.0 eq) in ethyl acetate (5 mL) was added HCl/ethyl acetate (10 ml, 3 N), then the mixture is stirred at room temperature for 4 h. The mixture was filtered, and then the solid was collected, dried in vacuum to give 1-[(4R)-8-[2-(trifluoromethyl)pyridin-4-yl]-3,4-dihydro-2H-1-benzopyran-4-yl]methanamine dihydrochloride (71 mg). MS (ESI): m/z 309 [M+H]⁺. ¹H NMR (400 MHz, CD₃OD) δ 8.72 (d, J=5.2 Hz, 1H), 7.98 (s, 1H), 7.81 (d, J=5.2 Hz, 1H), 7.41-7.37 (m, 2H), 7.11 (t, J=7.6 Hz, 1H), 4.33-4.27 (m, 2H), 3.43-3.39 (m, 1H), 3.32-3.22 (m, 2H), 2.28-2.22 (m, 1H), 2.11-2.05 (m, 1H).

Synthesis of tert-butyl (R)-methyl((8-(2-(trifluoromethyl)pyridin-4-yl)chroman-4-yl)methyl)carbamate: To a solution of tert-butyl N-{[(4R)-8-[2-(trifluoromethyl)pyridin-4-yl]-3,4-dihydro-2H-1-benzopyran-4-yl]methyl}carbamate (120 mg, 0.293 mmol, 1.0 eq) in N,N-dimethylformamide (1.5 mL) was added sodium hydride (29.1 mg, 0.732 mmol, 2.5 eq) then the mixture is stirred at RT for 30 min. Iodomethane (207 mg, 1.46 mmol, 5.0 eq) was added, then the mixture is stirred at room temperature overnight. Water (50 mL) was added, and then the mixture was extracted with ethyl acetate (50 mL×2), Combined the organic phase, and then the organic layer was washed with brine (50 mL), dried over anhydrous sodium sulfate and evaporated. The residue was purified by prep-HPLC to give the product (40 mg, yield 32.5%). MS (ESI): m/z 423 [M+H]⁺.

Synthesis of Compound 10: To a solution of tert-butyl N-methyl-N-{[(4R)-8-[2-(trifluoromethyl)pyridin-4-yl]-3,4-dihydro-2H-1-benzopyran-4-yl]methyl}carbamate (40 mg, 0.0946 mmol, 1.0 eq) in ethyl acetate (5 mL) was added HCl/ethyl acetate (10 ml, 3 N) then the mixture was stirred at room temperature for 4 h. The mixture was concentrated under reduced pressure, and then dried by freeze-drying on lyophilizer to give Compound 10 as a white solid (26.22 mg, purity 100%, yield 86.1%). MS (ESI): m/z 323 [M+H]⁺. ¹H NMR (400 MHz, CD₃OD) δ 8.72 (d, J=5.2 Hz, 1H), 7.97 (d, J=0.8 Hz, 1H), 7.81 (dd, J=1.2, 4.8 Hz, 1H), 7.43-7.37 (m, 2H), 7.11 (t, J=7.6 Hz, 1H), 4.34-4.28 (m, 2H), 3.46-3.34 (m, 3H), 2.83 (s, 3H), 2.29-2.22 (m, 1H), 2.12-2.05 (m, 1H).

### Example 5: Synthesis of Compound 11, Compound 12

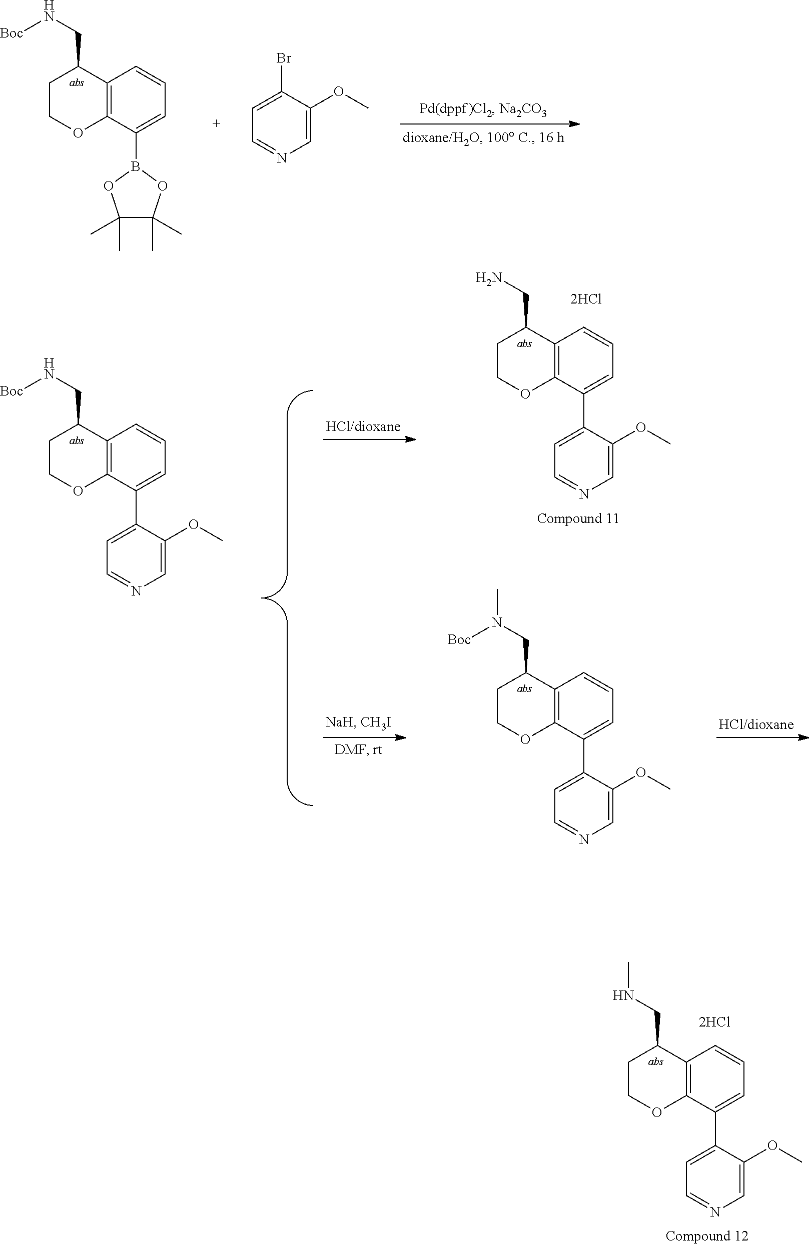

Synthesis of tert-butyl (R)-((8-(2,3-dimethylpyridin-4-yl)chroman-4-yl)methyl)carbamate: To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (400 mg, 1.02 mmol) and 4-bromo-3-methoxypyridine (210 mg, 1.12 mmol) in dioxane (10 mL) and water (2.5 mL) was added Na₂CO₃ (324 mg, 3.06 mmol) and [1,1′-Bis(diphenylphosphino)ferrocene]dichloropalladium(II) (86.0 mg, 102 μmol). The reaction was stirred at 100° C. for 16 h, and then concentrated to afford the crude product. The crude product was purified by flash column chromatography with an isocratic elution of petroleum ether (35%) and ethyl acetate (65%) to provide tertbutyl N-{[(4R)-8-(3-methoxypyridin-4-yl)-3,4-dihydro-2H-1-benzopyran-4-yl]methyl}carbamate (170 mg, 459 μmol, yield: 45.0%) as a yellow oil. MS (ESI): m/z 371 [M+H]⁺.

Synthesis of tert-butyl (R)-((8-(2,3-dimethylpyridin-4-yl)chroman-4-yl)methyl)carbamate dihydrochloride salt (Compound 11): To a solution of tert-butyl N-{[(4R)-8-(3-methoxypyridin-4-yl)-3,4-dihydro-2H-1-benzopyran-4-yl]methyl}carbamate (78 mg, 200 μmol) in methanol (3 mL) was added 4 N HCl/dioxane (1 mL, 4 mmol). The reaction was stirred at ambient temperature for 16 h, and then concentrated to provide [(4R)-8-(3-methoxypyridin-4-yl)-3,4-dihydro-2H-1-benzopyran-4-yl]methanamine dihydrochloride salt (76.0 mg) as a yellow solid. MS (ESI): m/z 271 [M+H]⁺. ¹H NMR (400 MHz, CD₃OD) δ 8.66 (s, 1H), 8.51 (d, J=5.7 Hz, 1H), 7.95 (d, J=5.7 Hz, 1H), 7.43 (d, J=7.7 Hz, 1H), 7.27 (d, J=7.0 Hz, 1H), 7.08 (t, J=7.6 Hz, 1H), 4.20 (dd, J=12.4, 6.8 Hz, 2H), 4.05 (s, 3H), 3.49-3.35 (m, 1H), 3.32 (m, 1H), 3.24-3.13 (m, 1H), 2.20 (m, 1H), 2.11-1.95 (m, 1H).

Synthesis of tert-butyl (R)-((8-(2,3-dimethylpyridin-4-yl)chroman-4-yl)methyl)carbamate: To a solution of tert-butyl N-{[(4R)-8-(3-methoxypyridin-4-yl)-3,4-dihydro-2H-1-benzopyran-4-yl]methyl}carbamate (100 mg, 264 μmol) in dimethylformamide (4 mL) was added sodium hydride (50 mg, 792 μmol) and iodomethane (56.2 mg, 396 μmol). The reaction was stirred at ambient temperature for 16 h. The mixture was purified by pre-HPLC with a gradient elution of water (100%) and acetonitrile (0%) to water (5%) and acetonitrile (95%) to provide 1-[(4R)-8-(3-methoxypyridin-4-yl)-3,4-dihydro-2H-1-benzopyran-4-yl]methanamine (150 mg, yield: 90%) as a white solid. MS (ESI): m/z 385 [M+H]⁺.

Synthesis of (R)-1-(8-(3-methoxypyridin-4-yl)chroman-4-yl)-N-methylmethanamine dihydrochloride salt (Compound 12): To a solution of tert-butyl N-{[(4R)-8-(3-methoxypyridin-4-yl)-3,4-dihydro-2H-1-benzopyran-4-yl]methyl}-N-methylcarbamate (117 mg, 304 μmol) in methanol (5 mL) was added 4N HCl/dioxane (1 mL, 4 mmol). The reaction was stirred at ambient temperature for 16 h. After concentration, the desired product (R)-1-(8-(3-methoxypyridin-4-yl)chroman-4-yl)-N-methylmethanamine dihydrochloride (59.5 mg, yield: 55.0%) as a white solid was obtained. MS (ESI): m/z 285 [M+H]⁺. ¹H NMR (400 MHz, CD₃OD): δ 8.76 (s, 1H), 8.54 (d, J=5.8 Hz, 1H), 7.90 (d, J=6.0 Hz, 1H), 7.43 (d, J=7.5 Hz, 1H), 7.27 (d, J=6.6 Hz, 1H), 7.07 (t, J=7.6 Hz, 1H), 4.44 (s, 3H), 4.19 (dd, J=17.6, 11.1 Hz, 2H), 4.03 (s, 3H), 3.40 (dd, J=12.4, 3.6 Hz, 1H), 3.35-3.31 (m, 1H), 3.27-3.10 (m, 1H), 2.19 (dt, J=13.2, 5.8 Hz, 1H), 2.14-1.95 (m, 1H).

### Example 6: Synthesis of Compound 13, Compound 14

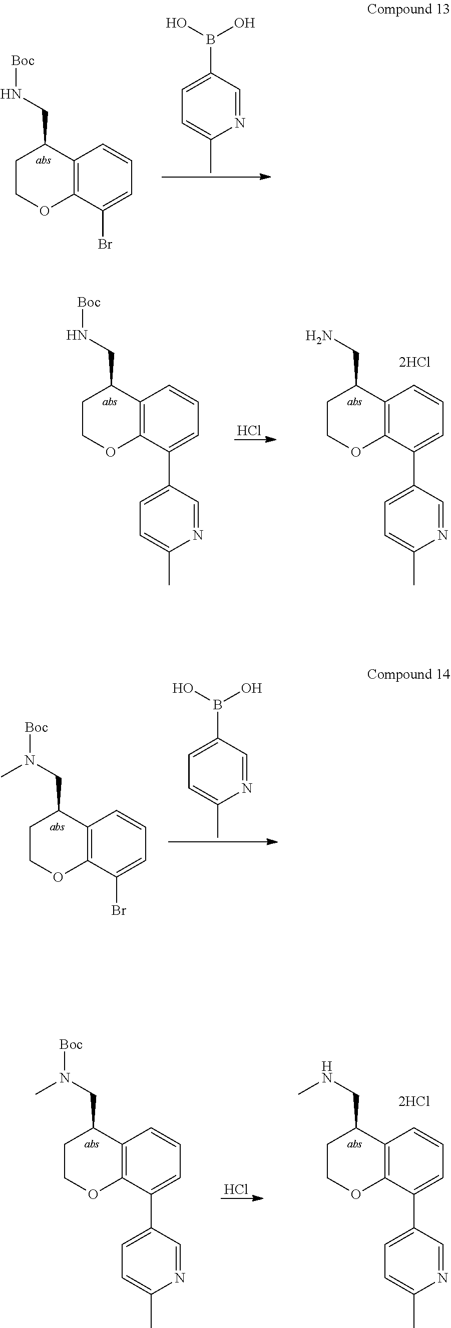

Synthesis of tert-butyl (R)-((8-(6-methylpyridin-3-yl)chroman-4-yl)methyl)carbamate: To a solution of tert-butyl (R)-((8-bromochroman-4-yl)methyl)carbamate (350 mg, 1.02 mmol) in 1,4-dioxane/water (10 ml) was added (6-methylpyridin-3-yl)boronic acid (167 mg, 1.22 mmol), [1,1′-Bis(diphenylphosphino)ferrocene]dichloropalladium(II) (74.6 mg, 102 μmol) and sodium carbonate (216 mg, 2.04 mmol) at rt under N₂ atmosphere. Then the mixture was stirred at 100° C. for 16 h under N₂ atmosphere. Water (20 mL) was added to the reaction vessel and the resulting biphasic mixture was transferred to a separatory funnel. The layers were separated and the aqueous phase was extracted with ethyl acetate (3×20 mL). The combined organics were dried over anhydrous sodium sulphate, filtered and concentrated in vacuo. The resulting mixture was purified by flash column chromatography with an isocratic elution of petroleum ether (100%) and ethyl acetate (0%) to petroleum ether (60%) and ethyl acetate (40%) to provide tert-butyl (R)-((8-(6-methylpyridin-3-yl)chroman-4-yl)methyl)carbamate as a colorless oil (250 mg). MS (ESI): m/z 355 [M+H]⁺.

Synthesis of (R)-(8-(6-methylpyridin-3-yl)chroman-4-yl)methanamine: To a solution of tert-butyl (R)-((8-(6-methylpyridin-3-yl)chroman-4-yl)methyl)carbamate (90 mg, 253 μmol) in ethyl acetate (3 mL) was added 3N HCl/ethyl acetate (3 mL, 9 mmol). Then the mixture was stirred at r.t. for 16 h. After concentration, the residue was triturated with heptane/ethyl acetate (3:1, 4 mL×3). The obtained solid was dried by freeze-drying on lyophilizer to give Compound 13 as a white solid (62 mg, purity: 100%, yield: 90%). MS (ESI): m/z 255 [M+H]⁺. ¹H NMR (500 MHz, CD₃OD): 8.89 (d, J=1.6 Hz, 1H), 8.66 (dd, J=6.4, 1.6 Hz, 1H), 7.69 (d, J=7.2 Hz, 1H), 7.45-7.43 (m, 2H), 7.15 (t, J=6.0 Hz, 1H), 4.33-4.30 (m, 2H), 3.44-3.32 (m, 3H), 2.84 (s, 3H), 2.26-2.23 (m, 1H), 2.12-2.08 (m, 1H).

Synthesis of tert-butyl (R)-methyl((8-(6-methylpyridin-3-yl)chroman-4-yl)methyl)carbamate: To a solution of tert-butyl (R)-((8-(6-methylpyridin-3-yl)chroman-4-yl)methyl)carbamate (160 mg, 0.449 mmol) in 1,4-dioxane/water (10 mL) was added (6-methylpyridin-3-yl)boronic acid (73.6 mg, 0.538 mmol), [1,1′-Bis(diphenylphosphino)ferrocene]dichloropalladium(II) (32.8 mg, 0.05 mmol) and sodium carbonate (95.1 mg, 0.898 mmol) at rt under N₂ atmosphere. Then the mixture was stirred at 100° C. for 16 h under N₂ atmosphere. Water (20 mL) was added to the reaction vessel, and the resulting biphasic mixture was transferred to a separatory funnel. The layers were separated, and the aqueous phase was extracted with ethyl acetate (3×20 mL). The combined organics were dried over anhydrous sodium sulphate, filtered and concentrated in vacuo. The resulting mixture was purified by flash column chromatography with an isocratic elution of petroleum ether (100%) and ethyl acetate (0%) to petroleum ether (60%) and ethyl acetate (40%) to provide tert-butyl (R)-methyl((8-(6-methylpyridin-3-yl)chroman-4-yl)methyl)carbamates a colorless oil (130 mg). MS (ESI): m/z 369 [M+H]⁺.

Synthesis of (R)—N-methyl-1-(8-(6-methylpyridin-3-yl)chroman-4-yl)methanamine: To a solution of tert-butyl (R)-methyl((8-(6-methylpyridin-3-yl)chroman-4-yl)methyl)carbamate (130 mg, 0.352 mmol) in ethyl acetate (3 mL) was added 3N HCl/ethyl acetate (3 mL, 9 mmol). Then the mixture was stirred at room temperature for 16 h. After concentration, the residue was triturated with heptane/ethyl acetate (3:1, 4 mL×3). The obtained solid was dried by freeze-drying on lyophilizer to give Compound 14 as a white solid (93 mg). MS (ESI): m/z 269 [M+H]⁺. ¹H NMR (500 MHz, CD₃OD): 8.89 (d, J=1.6 Hz, 1H), 8.66 (dd, J=6.4, 1.6 Hz, 1H), 7.69 (d, J=7.2 Hz, 1H), 7.45-7.43 (m, 2H), 7.15 (t, J=6.0 Hz, 1H), 4.33-4.30 (m, 2H), 3.44-3.32 (m, 3H), 2.85 (s, 3H), 2.84 (s, 3H), 2.26-2.23 (m, 1H), 2.12-2.08 (m, 1H).

### Example 7: Synthesis of Compound 15, Compound 16

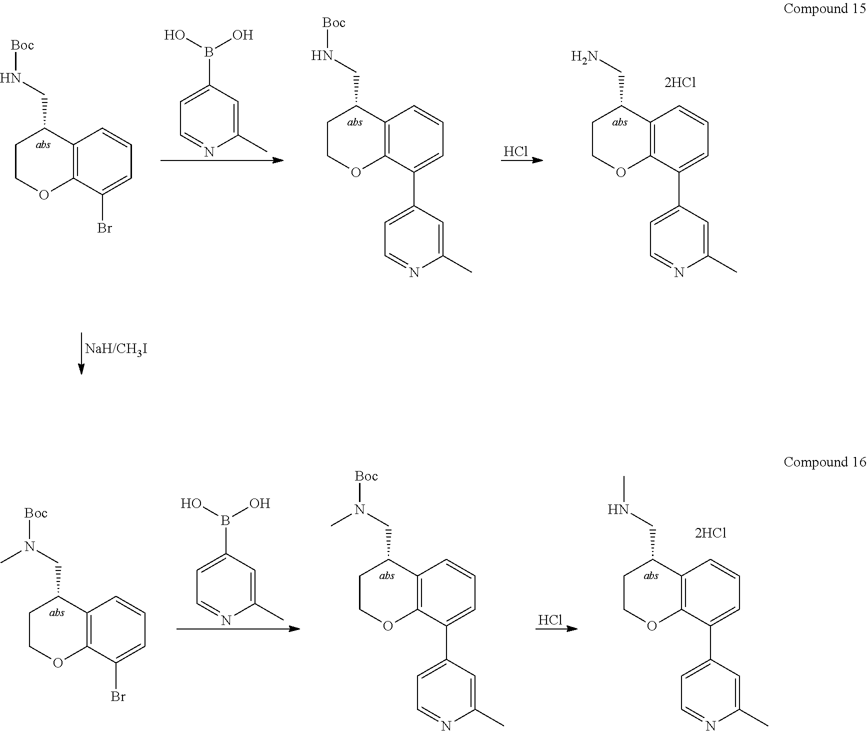

Synthesis of tert-butyl (S)-((8-(2-methylpyridin-4-yl)chroman-4-yl)methyl)carbamate: To a solution of tert-butyl (S)-((8-bromochroman-4-yl)methyl)carbamate (200 mg, 0.584 mmol) in 1,4-dioxane/water (4:1, 8 mL) was added (2-methylpyridin-4-yl)boronic acid (95.8 mg, 0.7 mmol), [1,1′-Bis(diphenylphosphino)ferrocene]dichloropalladium(II) (42.7 mg, 58.4 μmol) and sodium carbonate (122 mg, 1.16 mmol) at rt under N₂ atmosphere. Then the mixture was stirred at 100° C. for 16 h under N₂ atmosphere. Water (20 mL) was added to the reaction vessel and the resulting biphasic mixture was transferred to a separatory funnel. The layers were separated and the aqueous phase was extracted with ethyl acetate (3×20 mL). The combined organics were dried over anhydrous Na₂SO₄, filtered and concentrated in vacuo. The resulting mixture was purified by flash column chromatography with gradual elution of petroleum ether (100%) and ethyl acetate (0%) to petroleum ether (60%) and ethyl acetate (40%) to provide tert-butyl (S)-((8-(2-methylpyridin-4-yl)chroman-4-yl)methyl)carbamate as colorless oil (140 mg). MS (ESI): m/z 355 [M+H]⁺.

Synthesis of (S)-(8-(2-methylpyridin-4-yl)chroman-4-yl)methanamine: To a solution of tert-butyl (S)-((8-(2-methylpyridin-4-yl)chroman-4-yl)methyl)carbamate (140 mg, 0.394 mmol) in ethyl acetate (3 mL) was added 3N HCl/ethyl acetate (3 mL, 9 mmol). Then the mixture was stirred at rt for 16 h. After concentration, the residue was triturated with heptane/ethyl acetate (3:1, 4 ml×3). The obtained solid was dried by freeze-drying on lyophilizer to give Compound 15 as a white solid (90 mg purity: 100%, yield: 90%). MS (ESI): m/z 255 [M+H]⁺. ¹H NMR (500 MHz, CD₃OD) δ: 8.64 (d, J=5.2 Hz, 1H), 8.13-8.10 (m, 2H), 7.49 (dd, J=6.0, 2.4 Hz, 2H), 7.17 (t, J=6.0 Hz, 1H), 4.39-4.30 (m, 2H), 3.44-3.23 (m, 3H), 2.84 (s, 3H), 2.29-2.23 (m, 1H), 2.14-2.09 (m, 1H).

Synthesis of tert-butyl (S)-((8-bromochroman-4-yl)methyl)(methyl)carbamate: To a solution of tert-butyl (S)-((8-bromochroman-4-yl)methyl)carbamate (600 mg, 1.75 mmol) in N,N-dimethylformamide (5 mL) was added sodium hydride (210 mg, 5.25 mmol) at 0° C. After stirring at rt for 30 min, Iodomethane (620 mg, 4.37 mmol) was added to the mixture and stirred at rt for 3 h. Water (20 mL) was added to the reaction vessel and the mixture was extracted with ethyl acetate (3×20 mL). The combined organics were triturated with saturated sodium chloride solution (3×30 mL). The organics were dried over anhydrous sodium sulfate, filtered and concentrated in vacuo to get the crude product which was purified by flash column chromatography with elution of petroleum ether (100%) and ethyl acetate (0%) to petroleum ether (65%) and ethyl acetate (35%) to provide tert-butyl (S)-((8-bromochroman-4-yl)methyl)(methyl)carbamate as a colorless oil. 500 mg, MS (ESI): m/z 302 [M−55]⁺.

Synthesis of tert-butyl (S)-methyl((8-(2-methylpyridin-4-yl)chroman-4-yl)methyl)carbamate: To a solution of tert-butyl (S)-((8-bromochroman-4-yl)methyl)(methyl)carbamate (160 mg, 0.449 mmol) in 1,4-dioxane/water (10 mL) was added (2-methylpyridin-4-yl)boronic acid (61.4 mg, 0.449 mmol), [1,1′-Bis(diphenylphosphino)ferrocene]dichloropalladium(II) (32.8 mg, 0.449 mmol) and sodium carbonate (95.1 mg, 0.898 mmol) at rt under N₂ atmosphere. Then the mixture was stirred at 100° C. for 16 h under N₂ atmosphere. Water (20 mL) was added to the reaction vessel and the resulting biphasic mixture was transferred to a separatory funnel. The layers were separated and the aqueous phase was extracted with ethyl acetate (3×20 mL). The combined organics were dried over anhydrous sodium sulfate, filtered and concentrated in vacuo. The resulting mixture was purified by flash column chromatography with elution of petroleum ether (100%) and ethyl acetate (0%) to petroleum ether (60%) and ethyl acetate (40%) to provide tert-butyl (S)-methyl((8-(2-methylpyridin-4-yl)chroman-4-yl)methyl)carbamate as a colorless oil. 120 mg. MS (ESI): m/z 369 [M+H]⁺.

Synthesis of (S)—N-methyl-1-(8-(2-methylpyridin-4-yl)chroman-4-yl)methanamine: To a solution of tert-butyl (S)-methyl((8-(2-methylpyridin-4-yl)chroman-4-yl)methyl)carbamate (120 mg, 325 μmol) in ethyl acetate (3 mL) was added 3N HCl/ethyl acetate (3 mL, 9 mmol). Then the mixture was stirred at rt for 16 h. After concentration, the residue was triturated with heptane/ethyl acetate (3:1, 4 mL×3). The obtained solid was dried by freeze-drying on lyophilizer to get Compound 16 as a white solid (85 mg, purity: 100%, yield: 97%). MS (ESI): m/z 269 [M+H]⁺. ¹H NMR (500 MHz, CD₃OD): 8.65 (d, J=5.2 Hz, 1H), 8.14-8.11 (m, 2H), 7.54-7.52 (m, 2H), 7.13 (t, J=6.4 Hz, 1H), 4.39-4.30 (m, 2H), 3.48-3.23 (m, 3H), 2.84 (s, 6H), 2.29-2.23 (m, 1H), 2.14-2.09 (m, 1H).

### Example 8: Synthesis of Compound 17

### Synthesis of tert-butyl (R)-((8-(2-methylpyridin-4-yl)chroman-4-yl)methyl)carbamate

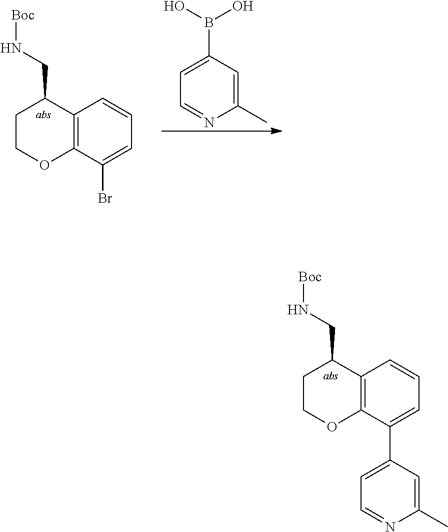

To a solution of tert-butyl (R)-((8-bromochroman-4-yl)methyl)carbamate (350 mg, 1.02 mmol) in 1,4-dioxane/water (8 mL) was added [1,1′-Bis(diphenylphosphino)ferrocene]dichloropalladium(II) (74.6 mg, 0.102 mmol), (2-methylpyridin-4-yl)boronic acid (167 mg, 1.22 mmol) and sodium carbonate (216 mg, 2.04 mmol) at rt under N₂ atmosphere. Then the mixture was stirred at 100° C. for 16 h under N₂ atmosphere. After cooling down to rt, water (10 mL) was added to the reaction vessel and the resulting biphasic mixture was transferred to a separatory funnel. The layers were separated and the aqueous phase was extracted with ethyl acetate (3×20 mL). The combined organics were dried over anhydrous sodium sulfate, filtered and concentrated in vacuo. The resulting mixture was purified by flash column chromatography with an isocratic elution of petroleum ether (100%) and ethyl acetate (0%) to petroleum ether (60%) and ethyl acetate (40%) to provide tert-butyl (R)-((8-(2-methylpyridin-4-yl)chroman-4-yl)methyl)carbamate as a colorless oil. 270 mg. Yield: 67%. MS (ESI): m/z 355 [M+H]⁺.

### Synthesis of (R)-(8-(2-methylpyridin-4-yl)chroman-4-yl)methanamine (Compound 17)

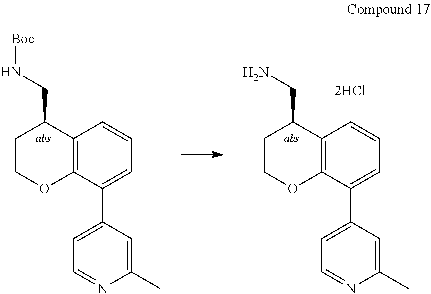 [SMILES: Cc1cc(-c2cccc3c2OCC[C@H]3CNC(=O)OC(C)(C)C)ccn1 ; Cc1cc(-c2cccc3c2OCC[C@H]3CN)ccn1]

To a solution of tert-butyl (R)-((8-(2-methylpyridin-4-yl)chroman-4-yl)methyl)carbamate (70 mg, 0.197 mmol) in ethyl acetate (3 mL) was added 3N HCl/ethyl acetate (3 mL). Then the mixture was stirred at rt for 16 h. After concentration, the residue was triturated with heptane/ethyl acetate (3:1, 4 mL×3). The obtained solid was dried by freeze-drying on lyophilizer to get Compound 17 as a white solid (41 mg, purity: 100%, yield: 81%). MS (ESI): m/z 255 [M+H]⁺. ¹H NMR (500 MHz, CD₃OD): 8.64 (d, J=5.2 Hz, 1H), 8.13-8.10 (m, 2H), 7.49 (dd, J=6.0, 2.4 Hz, 2H), 7.17 (t, J=6.0 Hz, 1H), 4.39-4.30 (m, 2H), 3.44-3.23 (m, 3H), 2.84 (s, 3H), 2.29-2.23 (m, 1H), 2.14-2.09 (m, 1H).

### Example 9: Synthesis of Compound 18

### Synthesis of tert-butyl (R)-methyl((8-(2-methylpyridin-4-yl)chroman-4-yl)methyl)carbamate

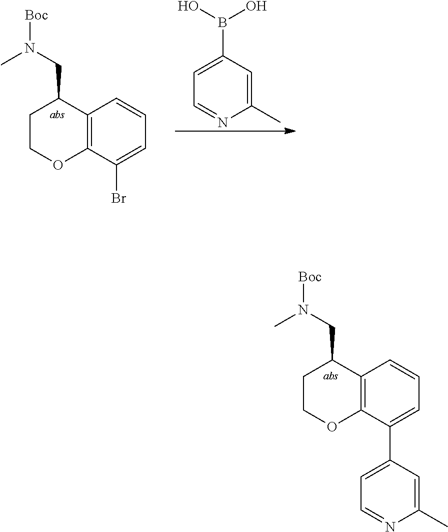

To a solution of tert-butyl (R)-((8-bromochroman-4-yl)methyl)(methyl)carbamate (180 mg, 0.505 mmol) in 1,4-dioxane/water (5 mL) was added (2-methylpyridin-4-yl)boronic acid (82.9 mg, 0.606 mmol), [1,1′-Bis(diphenylphosphino)ferrocene]dichloropalladium(II) (74.6 mg, 0.102 mmol), and sodium carbonate (107 mg, 1.01 mmol) at rt under N₂ atmosphere. Then the mixture was stirred at 100° C. for 16 h under N₂ atmosphere. Water (20 mL) was added to the reaction vessel and the resulting biphasic mixture was transferred to a separatory funnel. The layers were separated and the aqueous phase was extracted with ethyl acetate (3×20 mL). The combined organics were dried over anhydrous Na₂SO₄, filtered and concentrated in vacuo. The resulting mixture was purified by flash column chromatography with an isocratic elution of petroleum ether (100%) and ethyl acetate (0%) to petroleum ether (60%) and ethyl acetate (40%) to provide tert-butyl (R)-methyl((8-(2-methylpyridin-4-yl)chroman-4-yl)methyl)carbamate as a colorless oil. 150 mg. Yield: 80%. MS (ESI): m/z 369 [M+H]⁺.

### Synthesis of (R)—N-methyl-1-(8-(2-methylpyridin-4-yl)chroman-4-yl)methanamine (Compound 18)

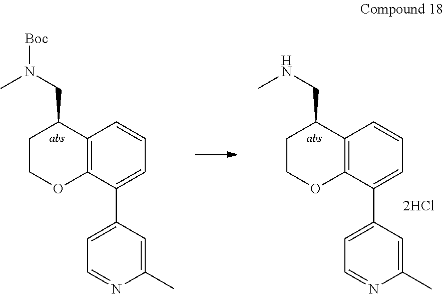

To a solution of tert-butyl (R)-((8-(2-methylpyridin-4-yl)chroman-4-yl)methyl)carbamate (150 mg, 0.407 mmol) in ethyl acetate (3 mL) was added 3N HCl/ethyl acetate (3 mL). Then the mixture was stirred at rt for 16 h. After concentration, the residue was triturated with heptane/ethyl acetate (3:1, 4 mL×3). The obtained solid was by freeze-drying on lyophilizer to get Compound 18 as a white solid (108 mg, purity: 100%, yield: 99%). MS (ESI): m/z 269 [M+H]⁺. ¹H NMR (500 MHz, CD₃OD): 8.65 (d, J=5.2 Hz, 1H), 8.14-8.11 (m, 2H), 7.54-7.52 (m, 2H), 7.13 (t, J=6.4 Hz, 1H), 4.39-4.30 (m, 2H), 3.48-3.23 (m, 3H), 2.84 (s, 6H), 2.29-2.23 (m, 1H), 2.14-2.09 (m, 1H).

### Example 10: Synthesis of Compound 19

### Synthesis of tert-butyl (R)-((8-(2,5-dimethylpyridin-4-yl)chroman-4-yl)methyl)carbamate

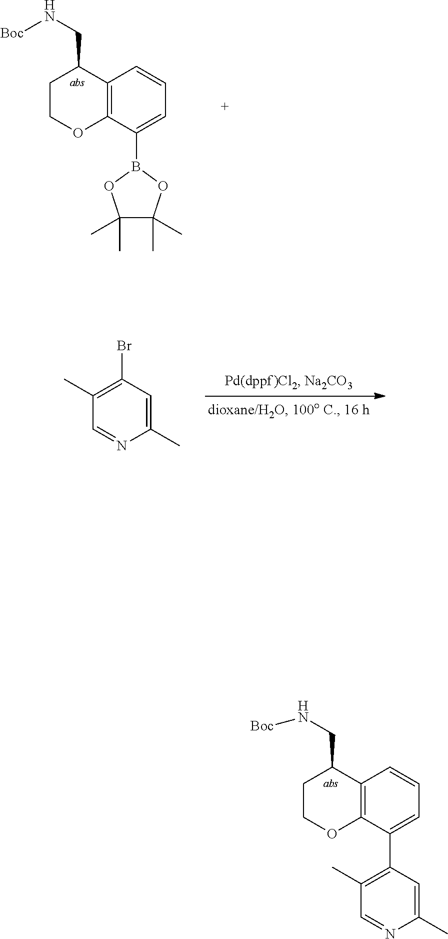

To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (400 mg, 1.02 mmol) and 4-bromo-2,5-dimethylpyridine (207 mg, 1.12 mmol) in dioxane (10 mL) was added water (2 mL), [1,1′-Bis(diphenylphosphino)ferrocene]dichloropalladium(II) (84.3 mg, 102 μmol) and sodium carbonate (324 mg, 3.06 mmol). The reaction was stirred at 100° C. for 16 h. Water (10 mL) was added to the reaction vessel and the resulting biphasic mixture was transferred to a separatory funnel. The layers were separated and the organic phase was extracted with ethyl acetate (3×15 mL) and saturated aqueous NaCl (2×10 mL). The combined organics were dried over anhydrous Na₂SO₄, filtered and concentrated in vacuo to afford crude product. The crude product was purified by flash column chromatography with an isocratic elution of petroleum ether (30%) and ethyl acetate (70%) to provide tert-butyl-N-{[(4R)-8-(2,5-dimethylpyridin-4-yl)-3,4-dihydro-2H-1-benzopyran-4-yl]methyl}carbamate (180 mg, 488 μmol, yield: 48%) as a yellow oil. MS (ESI): m/z=369.4.

### Synthesis of (R)-(8-(2,5-dimethylpyridin-4-yl)chroman-4-yl)methanamine dihydrochloride (Compound 19)

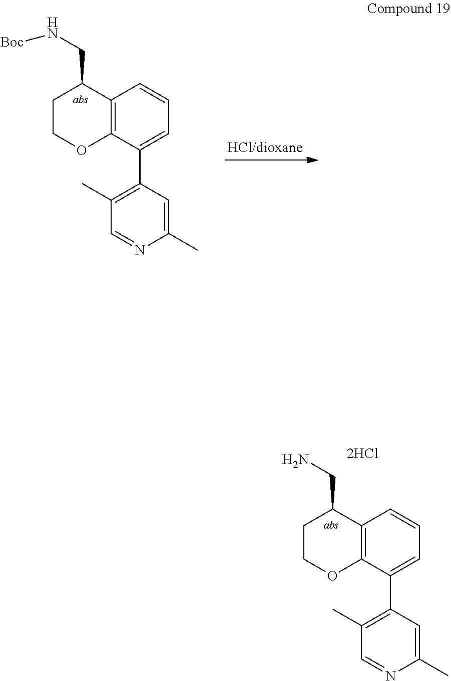 [SMILES: Cc1cc(-c2cccc3c2OCC[C@H]3CNC(=O)OC(C)(C)C)c(C)cn1 ; Cc1cc(-c2cccc3c2OCC[C@H]3CN)c(C)cn1]

To a solution of tert-butyl N-{[(4R)-8-(2,5-dimethylpyridin-4-yl)-3,4-dihydro-2H-1-benzopyran-4-yl]methyl}carbamate (50 mg, 132 μmol) in methanol (2 mL) was added 4 N HCl/dioxane (1 mL, 4 mmol). The reaction was stirred at ambient temperature for 16 h. The mixture was concentrated to provide (R)-(8-(2,5-dimethylpyridin-4-yl)chroman-4-yl)methanamine dihydrochloride (23.5 mg, yield: 52.2%) as a white solid. MS (ESI): m/z=269[M+H]⁺. ¹H NMR (400 MHz, CD3OD) δ 8.61 (s, 1H), 7.74 (s, 1H), 7.46 (d, J=6.1 Hz, 1H), 7.26-6.99 (m, 2H), 4.47-4.09 (m, 2H), 3.49-3.38 (m, 1H), 3.32 (s, 1H), 3.26-3.11 (m, 1H), 2.76 (s, 3H), 2.30 (s, 3H), 2.23-2.11 (m, 1H), 2.04 (dd, J=14.5, 4.2 Hz, 1H).

### Example 11. Synthesis of Compound 20

### Synthesis of tert-butyl N-{[(4R)-8-(3-methylpyridin-4-yl)-3,4-dihydro-2H-1-benzopyran-4-yl]methyl}carbamate

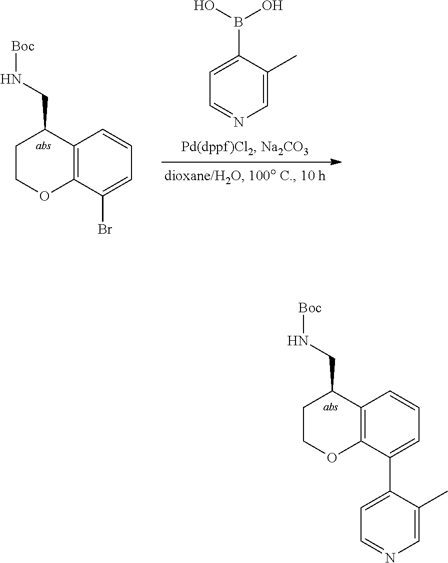

To a solution of tert-butyl N-{[(4R)-8-bromo-3,4-dihydro-2H-1-benzopyran-4-yl]methyl}-N-methylcarbamate (0.3 g, 0.876 mmol) in dioxane (10 mL) was added Pd(dppf)Cl₂ (64.2 mg, 0.0876 mmol), Na₂CO₃ (277 mg, 2.62 mmol) and (3-methylpyridin-4-yl)boronic acid (149 mg, 1.09 mmol). The reaction mixture was heated to 100° C. and stirred at that temperature for 24 h under nitrogen atmosphere. Ethyl acetate (30 mL) was added to the reaction vessel and the resulting biphasic mixture was transferred to a separatory funnel. The layers were separated and the organic phase was triturated with saturated aqueous NaCl (30 mL×4). The combined organics were dried over anhydrous Na₂SO₄, filtered and concentrated in vacuo. The resulting solid was purified by flash column chromatography with a gradient elution of petroleum ether (100%) to petroleum ether (70%) and ethyl acetate (30%) to provide tert-butyl N-{[(4R)-8-(3-methylpyridin-4-yl)-3,4-dihydro-2H-1-benzopyran-4-yl]methyl}carbamate (111 mg, yield: 37.2%) as a yellow oil. (ESI) m/z: 355[M+H]⁺.

### Synthesis of (R)-(8-(3-methylpyridin-4-yl)chroman-4-yl)methanamine (Compound 20)

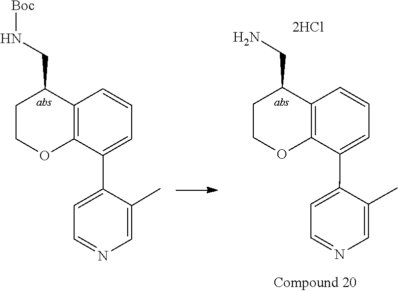 [SMILES: Cc1cnccc1-c1cccc2c1OCC[C@H]2CNC(=O)OC(C)(C)C ; Cc1cnccc1-c1cccc2c1OCC[C@H]2CN]

To a solution of tert-butyl N-{[(4R)-8-(3-methylpyridin-4-yl)-3,4-dihydro-2H-1-benzopyran-4-yl]methyl}carbamate (70 mg, 0.197 mmol) was added 3N HCl/ethyl acetate (10 mL, 30 mmol). The reaction was stirred at room temperature for 2 h. Upon the completion, the mixture was evaporated in vacuo to dryness and then dried by freeze dryer to yield Compound 20 (13.6 mg yield: 27.1%) as white solid. (ESI) m/z: 255 [M+H]⁺. 1H NMR (400 MHz, CD₃OD) δ 8.79 (s, H), 7.90 (d, J=5.6 Hz, 1H), 7.48 (d, J=7.2 Hz, 1H), 7.22-7.14 (m, 2H), 4.25-4.23 (m, 2H), 3.46-3.24 (m, 3H), 2.37 (s, 3H), 2.23-2.19 (m, 1H), 2.09-2.4 (m, 1H).

### Example 12: Synthesis of Compound 21

### Synthesis of tert-butyl (R)-methyl((8-(3-methylpyridin-4-yl)chroman-4-yl)methyl)carbamate

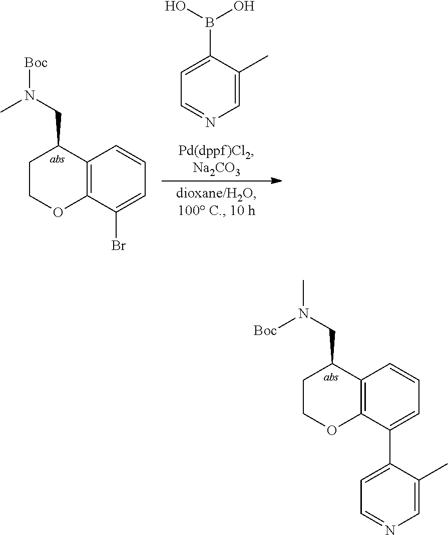

To a solution of tert-butyl N-{[(4R)-8-bromo-3,4-dihydro-2H-1-benzopyran-4-yl]methyl}-N-methylcarbamate (0.12 g, 0.336 mmol) in dioxane (10 mL) was added Pd(dppf)Cl₂ (24.5 mg, 0.0336 mmol), Na₂CO₃ (106 mg, 1 mmol) and (3-methylpyridin-4-yl)boronic acid (59.7 mg, 0.436 mmol). The reaction mixture was heated to 100° C. and stirred at that temperature for 24 h under nitrogen atmosphere. Ethyl acetate (30 mL) was added to the reaction vessel, and the resulting biphasic mixture was transferred to a separatory funnel. The layers were separated and the organic phase was triturated with saturated aqueous NaCl (4×30 mL). The combined organics were dried over anhydrous Na₂SO₄, filtered and concentrated in vacuo. The resulting solid was purified by flash column chromatography with a gradient elution of petroleum ether (100%) to petroleum ether (70%) and ethyl acetate (30%) to provide tert-butyl N-methyl-N-{[(4R)-8-(3-methylpyridin-4-yl)-3,4-dihydro-2H-1-benzopyran-4-yl]methyl}carbamate (61.2 mg, yield: 49.7%) as a yellow oil. (ESI) m/z: 367[M+H]⁺.

### Synthesis of methyl({[(4R)-8-(3-methylpyridin-4-yl)-3,4-dihydro-2H-1-benzopyran-4-yl]methyl})amine (Compound 21)

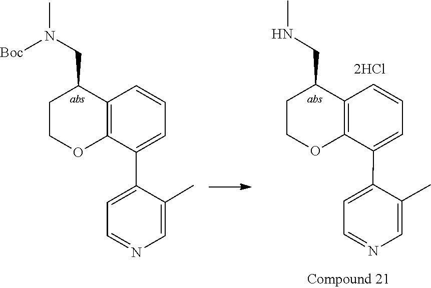 [SMILES: Cc1cnccc1-c1cccc2c1OCC[C@H]2CN(C)C(=O)OC(C)(C)C ; CNC[C@@H]1CCOc2c(-c3ccncc3C)cccc21]

To a solution of tert-butyl N-methyl-N-{[(4R)-8-(3-methylpyridin-4-yl)-3,4-dihydro-2H-1-benzopyran-4-yl]methyl}carbamate (90 mg, 0.197 mmol) was added 3N HCl/ethyl acetate (5 mL) The reaction was stirred at room temperature for 2 h. Upon the completion, the mixture was evaporated in vacuo to dryness and then dried on freeze dryer to yield Compound 21 (42.7 mg yield: 65.2%) as white solid. (ESI) m/z: 269 [M+H]⁺. ¹H NMR (400 MHz, CD₃OD) δ 9.02-8.45 (m, 2H), 7.89 (d, J=6.0 Hz, 1H), 7.50 (dd, J=7.6, 1.5 Hz, 1H), 7.31-6.97 (m, 2H), 4.20 (dt, J=48.2, 24.1 Hz, 2H), 3.62-3.34 (m, 3H), 2.91 (d, J=70.7 Hz, 3H), 2.65-1.71 (m, 5H).

### Example 13: Synthesis of Compound 22, Compound 23, Compound 24 and Compound 25

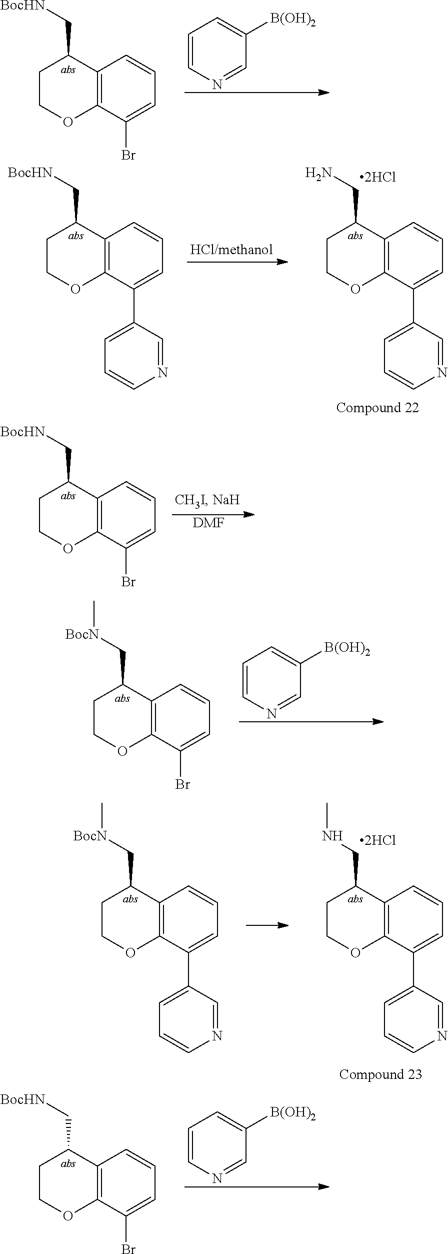

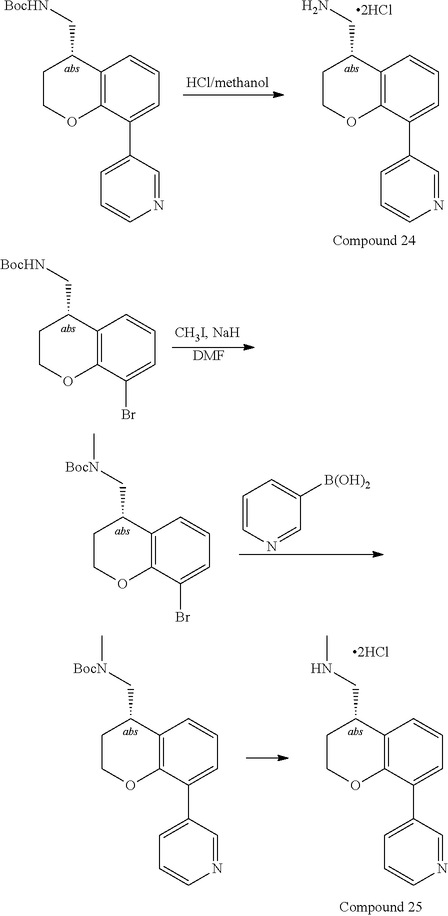

Synthesis of tert-butyl (R)-((8-(pyridin-3-yl) chroman-4-yl) methyl)carbamate: To a solution of tert-butyl (R)-((8-bromochroman-4-yl)methyl)carbamate (0.342 g, 999 μmol) in 1,4-dioxane/water (4:1, 6.5 mL) was added (pyridin-3-yl)boronic acid (183 mg, 1.49 mmol), sodium carbonate (316 mg, 2.99 mmol) and 1,1′-Bis(diphenylphosphino)ferrocene-palladium(II) (81.5 mg, 99.8 μmol). The reaction mixture was heated to 100° C., and stirred at that temperature for 16 h. Upon the completion, ethyl acetate (20 mL) and water (10 mL) were added to the mixture and the organic phase was washed with water (50 mL), dried and concentrated. The crude product was purified by flash column chromatography with a gradient elution of petroleum ether (100%) to petroleum ether (65%) and ethyl acetate (35%) to provide tert-butyl (R)-((8-(pyridin-3-yl)chroman-4-yl)methyl)carbamate (210 mg, yield: 61.9%) as a colorless oil. ESI: m/z=341[M+H]⁺.

Synthesis of Compound 22: A solution of tert-butyl (R)-((8-(pyridin-3-yl) chroman-4-yl) methyl)carbamate (0.06 g, 176 μmol) in HCl/methanol (4M, 2 mL) was stirred at ambient temperature for overnight. Upon the completion, the solvent was removed, and the solid was dried by freeze dryer to get Compound 22 as an off white solid (46.39 mg, yield: 84%). (ESI): m/z=241[M+H]⁺. ¹H NMR (400 MHz, CD₃OD) δ 9.11 (d, J=1.8 Hz, 1H), 8.88-8.79 (m, 2H), 8.17 (dd, J=8.0, 6 Hz, 1H), 7.51-7.43 (m, 2H), 7.17 (t, J=7.6 Hz, 1H), 4.34-4.30 (m, 2H), 3.45-3.36 (m, 2H), 3.29-3.19 (m, 1H), 2.32-2.19 (m, 1H), 2.14-2.08 (m, 1H).

Synthesis of tert-butyl (R)-methyl((8-(pyridin-3-yl)chroman-4-yl)methyl)carbamate: To a solution of tert-butyl (R)-((8-bromochroman-4-yl)methyl)(methyl)carbamate (0.25 g, 701 μmol) in 1,4-dioxane/water (4:1, 6.5 mL) was added (pyridin-3-yl)boronic acid (103 mg, 841 μmol), sodium carbonate (222 mg, 2.10 mmol) and 1,1′-Bis(diphenylphosphino)ferrocene-palladium(II)dichloride dichloromethane complex (57.2 mg, 70.1 μmol). The reaction mixture was heated to 100° C. and stirred at that temperature for 16 h. Upon the completion, ethyl acetate (50 mL) was added and the mixture was washed with water (50 mL), dried and concentrated. The crude product was purified by flash column chromatography with a gradient elution of petroleum ether (100%) to petroleum ether (70%) and ethyl acetate (30%) to provide tert-butyl (R)-methyl((8-(pyridin-3-yl)chroman-4-yl)methyl)carbamate (150 mg, yield: 60.4%) as a colorless oil. ESI: m/z=356[M+H]⁺.

Synthesis of Compound 23: A solution of tert-butyl (R)-methyl((8-(pyridin-3-yl)chroman-4-yl)methyl)carbamate (0.15 g, 423 μmol) in HCl/methanol (4M, 20 mL) was stirred at rt for overnight. Upon the completion, the solvent was removed and the solid was dried by freeze dryer to get Compound 23 as an off white solid (117.66 mg). (ESI): m/z=256[M+H]⁺. ¹H NMR (400 MHz, CD₃OD) δ 9.11 (d, J=1.4 Hz, 1H), 8.84 (dd, J=10, 3.8 Hz, 2H), 8.18 (dd, J=8.2, 6.0 Hz, 1H), 7.56-7.41 (m, 2H), 7.17 (t, J=7.8 Hz, 1H), 4.40-4.25 (m, 2H), 3.45-3.36 (m, 3H), 2.84 (s, 3H), 2.30-2.24 (m, 1H), 2.19-2.08 (m, 1H).

Synthesis of tert-butyl (S)-((8-(pyridin-3-yl) chroman-4-yl)methyl)carbamate: To a solution of tert-butyl (S)-((8-bromochroman-4-yl)methyl)carbamate (0.3 g, 876 μmol) in 1,4-dioxane/water (4:1, 7.5 mL) was added (pyridin-3-yl)boronic acid (129 mg, 1.05 mmol), sodium carbonate (277 mg, 2.62 mmol) and 1,1′-Bis(diphenylphosphino)ferrocene-palladium(II)dichloride dichloromethane complex (71.5 mg, 87.6 μmol). The reaction mixture was heated to 100° C. and stirred at that temperature for 16 h under N2 atmosphere. Upon the completion, ethyl acetate (50 mL) was added and the mixture was washed with water (50 mL), dried and concentrated. The resulting oil was purified by flash column chromatography with a gradient elution of petroleum ether (100%) to petroleum ether (70%) and ethyl acetate (30%) to provide tert-butyl (S)-((8-(pyridin-3-yl)chroman-4-yl)methyl)carbamate (170 mg, yield: 57%) as a colorless oil. ESI: m/z=341[M+H]⁺.

Synthesis of Compound 24: A solution of tert-butyl (S)-((8-(pyridin-3-yl)chroman-4-yl)methyl)carbamate (0.17 g, 499 μmol) in HCl/methanol (4M, 20 mL) was stirred at rt for overnight. Upon the completion, the solvent was removed and the solid was dried by freeze dryer. Compound 24 as an off white solid (130.84 mg, yield: 82.6%) was obtained. (ESI): m/z=241[M+H]⁺. ¹H NMR (400 MHz, CD₃OD) δ 9.11 (d, J=1.2 Hz, 1H), 8.84-8.81 (m, 2H), 8.14 (dd, J=8.0, 6 Hz, 1H), 7.48-7.39 (m, 2H), 7.14 (t, J=7.6 Hz, 1H), 4.36-4.24 (m, 2H), 3.41 (dd, J=12.6, 4.1 Hz, 1H), 3.37˜3.33 (m, 1H), 3.26-3.18 (m, 1H), 2.28-2.22 (m, 1H), 2.13-2.08 (m, 1H).

Synthesis of tert-butyl (S)-methyl((8-(pyridin-3-yl)chroman-4-yl)methyl)carbamate: To a solution of tert-butyl (S)-((8-bromochroman-4-yl)methyl)(methyl)carbamate (0.25 g, 701 μmol) in 1,4-dioxane/water (4:1, 6.5 mL) was added (pyridin-3-yl)boronic acid (103 mg, 841 μmol), sodium carbonate (222 mg, 2.10 mmol) and 1,1′-Bis(diphenylphosphino)ferrocene-palladium(II)dichloride dichloromethane complex (57.2 mg, 70.1 μmol). The reaction mixture was heated to 100° C. and stirred at that temperature for 16 h. Upon the completion, ethyl acetate was added and the mixture was washed with water (2×50 mL), dried and concentrated. The resulting oil was purified by flash column chromatography with a gradient elution of petroleum ether (100%) to petroleum ether (70%) and ethyl acetate (30%) to provide tert-butyl (S)-methyl((8-(pyridin-3-yl)chroman-4-yl)methyl)carbamate (150 mg, yield: 60.4%) as a colorless oil. ESI: m/z=356[M+H]⁺.

Synthesis of Compound 25: A solution of tert-butyl (S)-methyl((8-(pyridin-3-yl)chroman-4-yl)methyl)carbamate (0.15 g, 423 μmol) in HCl/methanol (4M, 20 mL) was stirred at rt for overnight. Upon the completion, the solvent was removed and the solid was dried by freeze dryer. Compound 25 as an off white solid (66.45 mg, ee %: 100%, yield: 48.1%) was obtained. (ESI): m/z=256[M+H]⁺. ¹H NMR (400 MHz, CD₃OD) δ 9.10 (d, J=2 Hz, 1H), 8.86-8.74 (m, 2H), 8.14 (dd, J=8.2, 6.0 Hz, 1H), 7.52-7.40 (m, 2H), 7.14 (t, J=7.8 Hz, 1H), 4.39-4.23 (m, 2H), 3.49-3.32 (m, 3H), 2.84 (s, 3H), 2.30-2.24 (m, 1H), 2.16-2.10 (m, 1H).

### Example 14: Synthesis of Compound 26

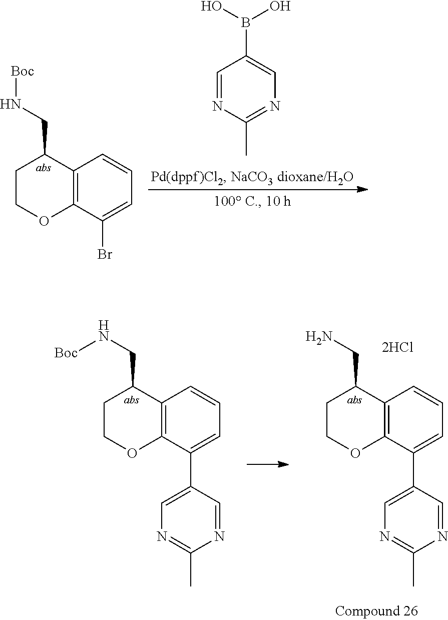

Synthesis of (R)-tert-butyl (8-(2-methylpyrimidin-5-yl)chroman-4-yl)methylcarbamate: A solution of tert-butyl (R)-((8-bromochroman-4-yl)methyl)carbamate (200 mg, 0.586 mmol), (2-methylpyrimidin-5-yl)boronic acid (0.08 g, 0.58 mmol, 1.0 equiv.), 1,1′-Bis(diphenylphosphino)ferrocene-palladium(II)dichloride dichloromethane complex (0.06 g, 0.087 mmol, 0.15 equiv.) and Na₂CO₃ (0.15 g, 1.45 mmol, 2.5 equiv) in dioxane/water (20 mL, 4:1) was stirred at 100° C. for 10 h. The reaction mixture was concentrated, washed with water (50 mL), extracted with ethyl acetate (100 mL×3), dried and concentrated, and purified by silica gel column (petrol ether/ethyl acetate=3/1) to give (R)-tert-butyl (8-(2-methylpyrimidin-5-yl)chroman-4-yl)methylcarbamate (0.1 g, yield: 48%). (ESI): m/z=356[M+H]⁺.

Synthesis of Compound 26: To a solution of (R)-tert-butyl (8-(2-methylpyrimidin-5-yl)chroman-4-yl)methylcarbamate (0.1 g, 0.28 mmol) in HCl/dioxane (20 mL, 4N) was stirred at rt for 3 h, monitored by LCMS, it was concentrated to get the crude, which was triturated with ethyl acetate (5 mL) to get Compound 26 (30 mg, yield: 42%). (ESI): m/z=256 [M+H]⁺. ¹H NMR (400 MHz, CD₃OD) δ 8.83 (s, 2H), 7.36-7.30 (m, 2H), 7.08 (t, J=8.0, 1 Hz, 1H), 4.29-4.25 (m, 2H), 3.37-3.33 (m, 1H), 3.27-3.30 (m, 1H), 3.17-3.14 (m, 1H), 2.74 (s, 3H), 2.24-2.17 (m, 1H), 2.09-2.04 (m, 1H).

### Example 15: Synthesis of Compound 27 and Compound 28

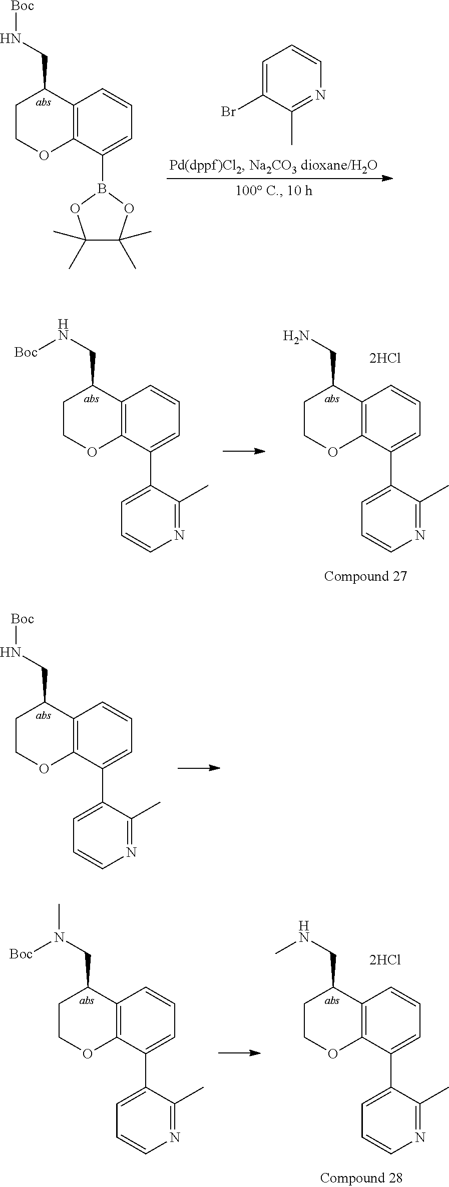

Synthesis of (R)-tert-butyl (8-(2-methylpyridin-3-yl)chroman-4-yl)methylcarbamate: A solution of 3-bromo-2-methylpyridine (0.09 g, 0.51 mmol, 1.0 equiv.), 1,1′-Bis(diphenylphosphino)ferrocene-palladium(II)dichloride dichloromethane complex 0.056 g, 0.077 mmol, 0.15 equiv.), Na₂CO₃ (0.13 g, 1.27 mmol, 2.5 equiv) and tert-butyl N-{[(4R)-8-(4,4,5,5-tetramethyl-1,3,2-dioxaborolan-2-yl)-3,4-dihydro-2H-1-benzopyran-4-yl]methyl}carbamate (0.2 g, 0.51 mmol, 1.0 equiv.) in dioxane/water (20 mL, 4:1) was stirred at 100° C. for 10 h. The reaction mixture was concentrated, and then water (50 mL) was added. The mixture was extracted with ethyl acetate (100 mL), dried and concentrated to get the crude product which was purified by silica gel column (petrol ether/ethyl acetate=3/1) to give (R)-tert-butyl (8-(2-methylpyridin-3-yl)chroman-4-yl)methylcarbamate (0.1 g, yield: 83%). (ESI): m/z=355[M+H]⁺

Synthesis of Compound 27: To a solution of (R)-tert-butyl (8-(2-methylpyridin-3-yl)chroman-4-yl)methylcarbamate (0.15 g, 0.42 mmol) in HCl/dioxane (20 mL, 4N) was stirred at rt for 3 h. It was concentrated to get the crude product which was triturated with ethyl acetate (5 mL), the solid was collected and dried to get Compound 27, 35 mg, yield: 33%. (ESI): m/z=255 [M+H]⁺. ¹H NMR (400 MHz, DMSO-d6) δ 8.78-8.76 (m, 1H), 8.43 (bs, 3H), 8.35 (d, J=8.0 Hz, 1H), 7.95-7.93 (m, 1H), 7.47 (d, J=7.5 Hz, 1H), 7.20-7.18 (m, 1H), 7.07 (t, J=7.5 Hz, 1H), 4.15 (t, J=5.0 Hz, 2H), 3.32-3.30 (m, 1H), 3.24-3.20 (m, 1H), 3.08 (bs, 1H), 2.58 (s, 3H), 2.15-2.11 (m, 1H), 2.04-2.0 (m, 1H).

Synthesis of (R)-tert-butyl methyl((8-(2-methylpyridin-3-yl)chroman-4-yl)methyl)carbamate: To a solution of (R)-tert-butyl (8-(2-methylpyridin-3-yl)chroman-4-yl)methylcarbamate (0.2 g, 0.56 mmol, 1.0 eq) in N,N-dimethylformamide (20 mL) was added sodium hydride (33 mg, 0.84 mmol, 1.5 eq, 60%), it was stirred at rt for 0.5 h, then iodomethane (0.16 g, 1.12 mmol, 2.0 eq) was added to the mixture. It was stirred for 3 h, monitored by TLC, and then the mixture was added ethyl acetate (50×2 mL). The mixture was washed with water (50 mL). The combined organic layer was washed with brine, dried over sodium sulfate, filtered and then concentrated in vacuum. The crude product was purified by chromatography on silica gel (petrol ether: ethyl acetate=3:1) to give (R)-tert-butyl methyl((8-(2-methylpyridin-3-yl)chroman-4-yl)methyl)carbamate (0.15 g, yield: 72%). (ESI): m/z=369 [M+H]⁺

Synthesis of Compound 28: To a solution of (R)-tert-butyl methyl((8-(2-methylpyridin-3-yl)chroman-4-yl)methyl)carbamate (0.15 g, 0.41 mmol) in HCl/dioxane (20 mL, 4N) was stirred at rt for 3 h, it was concentrated to get the crude product which was triturated with ethyl acetate (5 mL), the solid was collected and then dried to get Compound 28 (62 mg, yield: 57%). (ESI): m/z=269 [M+H]⁺. ¹H NMR (400 MHz, DMSO-d6) δ 9.13-9.07-8.76 (bs, 2H), 8.76 (d, J=4.8 Hz, 1H), 8.28 (d, J=7.0 Hz, 1H), 7.88 (t, J=6.8 Hz, 1H), 7.47 (d, J=6.4 Hz, 1H), 7.19-7.17 (m, 1H), 7.08 (t, J=7.2 Hz, 1H), 4.20-4.13 (m, 2H), 3.38-3.32 (m, 1H), 3.27-3.20 (m, 2H), 2.67 (t, J=3.2 Hz, 3H), 2.53 (s, 3H), 2.17-2.14 (m, 1H), 2.07-2.04 (m, 1H).

### Example 16: Synthesis of Compound 29

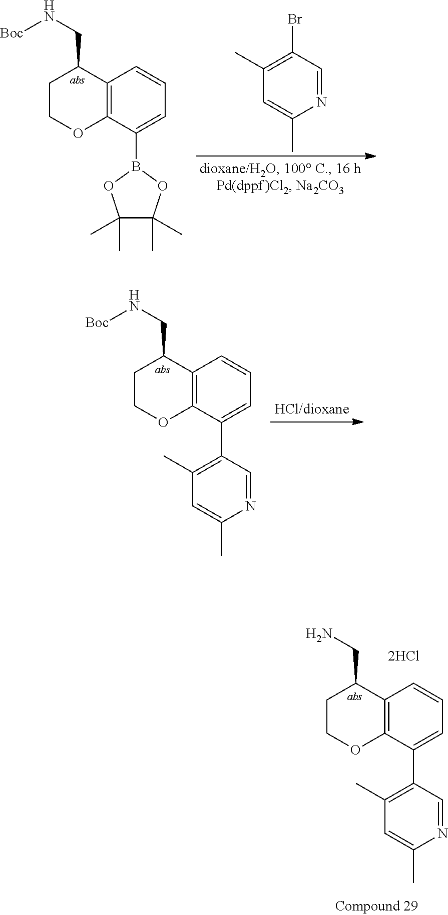

Synthesis of tert-butyl (R)-((8-(4,6-dimethylpyridin-3-yl)chroman-4-yl)methyl)carbamate: To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (400 mg, 1.02 mmol, 1.0 eq) and 4-5-bromo-2,4-dimethylpyridine (210 mg, 1.12 mmol, 1.1 eq) in 1,4-dioxane (10 mL)/water (2 ml) was added 1,1′-Bis(diphenylphosphino)ferrocene-palladium(II)(41.7 mg, 0.051 mmol, 0.05 eq) and sodium carbonate (324 mg, 3.06 mmol, 3.0 eq). Then the mixture was stirred at 100° C. for 16 h. The mixture was concentrated in vacuum and then purified by silica gel column (dichloromethane/methanol=30/1) to give the crude product which was purified by reverse phase (water/CH₃CN/NH₄HCO₃=65:35:0.1) to give the product (250 mg, yield 66.6%). (ESI): m/z=369.1[M+H]⁺

Synthesis of Compound 29: To a solution of tert-butyl N-{[(4R)-8-(4,6-dimethylpyridin-3-yl)-3,4-dihydro-2H-1-benzopyran-4-yl]methyl}carbamate (60 mg, 0162 mmol, 1.0 eq) in EtOAc (5 mL) was added 1,4-dioxane/HCl (10 ml, 4 N), then the mixture was stirred at R.T. for overnight. The mixture was concentrated under reduced pressure. The crude product was triturated in petrol ether (20 mL) with ultrasonic wave for 5 minutes. The solid was collected and then dried on lyophilizer to give the desired product hydrochloride1-[(4R)-8-(4,6-dimethylpyridin-3-yl)-3,4-dihydro-2H-1-benzopyran-4-yl]methanamine (35.2 mg, purity 98.04%, yield 79.4%) as a white solid. (ESI): m/z=269.3[M+H]⁺. ¹H NMR (400 MHz, CD₃OD) δ 8.46 (s, 1H), 7.87 (s, 1H), 7.46 (dd, J=1.2, 7.6 Hz, 1H), 7.20 (dd, J=1.8, 7.4 Hz, 1H), 7.13 (t, J=7.6 Hz, 1H), 4.23-4.21 (m, 2H), 3.46-3.42 (m, 1H), 3.35-3.33 (m, 1H), 3.26-3.20 (m, 1H), 2.80 (s, 3H), 2.44 (s, 3H), 2.22-2.18 (m, 1H), 2.08-2.03 (m, 1H).

### Example 17: Synthesis of Compound 30

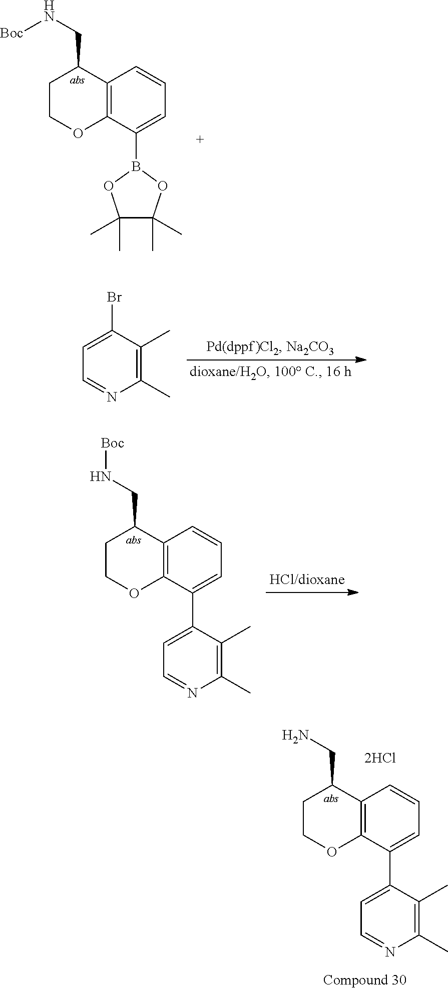

Synthesis of tert-butyl (R)-((8-(2,3-dimethylpyridin-4-yl)chroman-4-yl)methyl)carbamate: To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (400 mg, 1.02 mmol) and 4-bromo-2,3-dimethylpyridine (218 mg, 1.12 mmol) in dioxane (10 mL) and water (2.5 mL) was added Na₂CO₃ (324 mg, 3.06 mmol) and 1,1′-Bis(diphenylphosphino)ferrocene-palladium(II)dichloride dichloromethane complex (84.2 mg, 102 μmol). The reaction was stirred at 100° C. for 16 h. Water (10 mL) and EtOAc (10 mL) were added to the reaction vessel and the resulting biphasic mixture was transferred to a separatory funnel. The layers were separated and the organic phase was washed with saturated aqueous NaCl (2×15 mL). The combined organics were dried over anhydrous Na₂SO₄, filtered and concentrated in vacuo to afford crude product which was purified by flash column chromatography with an isocratic elution of petroleum ether (30%) and ethyl acetate (70%) to obtain tert-butyl N-{[(4R)-8-(2,3-dimethylpyridin-4-yl)-3,4-dihydro-2H-1-benzopyran-4-yl]methyl}carbamate (223 mg, yield: 59.4%) as a yellow oil. MS (ESI): m/z=369 [M+H]⁺.

Synthesis of (R)-(8-(2,3-dimethylpyridin-4-yl)chroman-4-yl)methanamine dihydrochloride (Compound 30): To a solution of tert-butyl N-{[(4R)-8-(2,3-dimethylpyridin-4-yl)-3,4-dihydro-2H-1-benzopyran-4-yl]methyl}carbamate (100 mg, 257 μmol) in methanol (3 mL) was added 4N HCl/dioxane (2 mL, 8 mmol). The reaction was stirred at ambient temperature for 6 h. After concentration, the desired product 1-[(4R)-8-(2,3-dimethylpyridin-4-yl)-3,4-dihydro-2H-1-benzopyran-4-yl]methanamine dihydrochloride salt (46.4 mg, 136 μmol, yield: 52.9%) as a white solid was obtained. MS (ESI): m/z=269[M+H]⁺. ¹H NMR (400 MHz, CD₃OD) δ 8.50 (d, J=6.1 Hz, 1H), 7.66 (d, J=5.9 Hz, 1H), 7.45 (dd, J=7.3, 1.7 Hz, 1H), 7.28-7.02 (m, 2H), 4.20 (d, J=3.9 Hz, 2H), 3.49-3.38 (m, 1H), 3.31 (s, 1H), 3.23 (s, 1H), 2.78 (s, 3H), 2.27 (s, 3H), 2.18 (s, 1H), 2.07-1.99 (m, 1H).

### Example 18: Synthesis of Compound 31

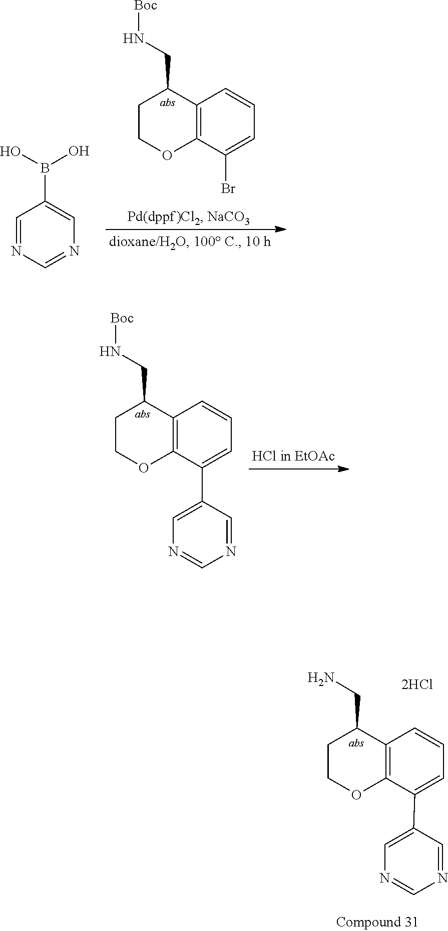

Synthesis of tert-butyl (R)-((8-(pyrimidin-5-yl)chroman-4-yl)methyl)carbamate: A solution of tert-butyl (R)-((8-bromochroman-4-yl)methyl)carbamate (200 mg, 0.5843 mmol) and (pyrimidin-5-yl)boronic acid (86.8 mg, 701 μmol) in dioxane (5 mL) and water (1 mL) was added 1,1′-Bis(diphenylphosphino)ferrocene-palladium(II)dichloride dichloromethane complex (95.8 mg, 116 μmol) and Na₂CO₃ (185 mg, 1.75 mmol). The reaction was stirred at 90° C. under argon for 16 h. Water (10 mL) and ethyl acetate (10 mL) was added to the reaction vessel and the resulting biphasic mixture was transferred to a separatory funnel. The layers were separated and the organic phase was washed with saturated aqueous NaCl (2×15 mL). The combined organics were dried over anhydrous Na₂SO₄, filtered and concentrated in vacuo to afford crude product. The crude product was purified by flash column chromatography with a gradient elution of petroleum ether (100%) to petrol ether/ethyl acetate=1:1 to provide tert-butyl (R)-((8-(pyrimidin-5-yl)chroman-4-yl)methyl)carbamate (143 mg, yield: 71.8%) as a yellow oil. MS(ESI): m/z=342 [M+H]⁺.

Synthesis of Compound 31: To a solution of tert-butyl (R)-((8-(pyrimidin-5-yl)chroman-4-yl)methyl)carbamate (146 mg, 0.4276 mmol) in ethyl acetate (5 mL) was added HCl in ethyl acetate (10 mL, 3M, 30.0 mmol). The reaction was stirred at ambient temperature for 3 h. The reaction was filtered, and the solid was triturated with ethyl acetate (10 mL) and then dried by freeze drying to provide Compound 31 (98.4 mg) as a white solid. MS(ESI): m/z 242 [M+H]⁺. ¹H NMR (400 MHz, DMSO) δ 9.15 (s, 1H), 8.94 (s, 2H), 8.39 (d, J=22.6 Hz, 3H), 7.53 (s, 1H), 7.40 (d, J=7.6 Hz, 1H), 7.36-7.27 (m, 1H), 7.05 (t, J=7.6 Hz, 1H), 4.29-4.11 (m, 2H), 3.28 (d, J=4.7 Hz, 1H), 3.19 (d, J=5.4 Hz, 1H), 3.07 (d, J=5.9 Hz, 1H), 2.28-2.08 (m, 1H), 2.08-1.94 (m, 1H).

### Example 19: Synthesis of Compound 32

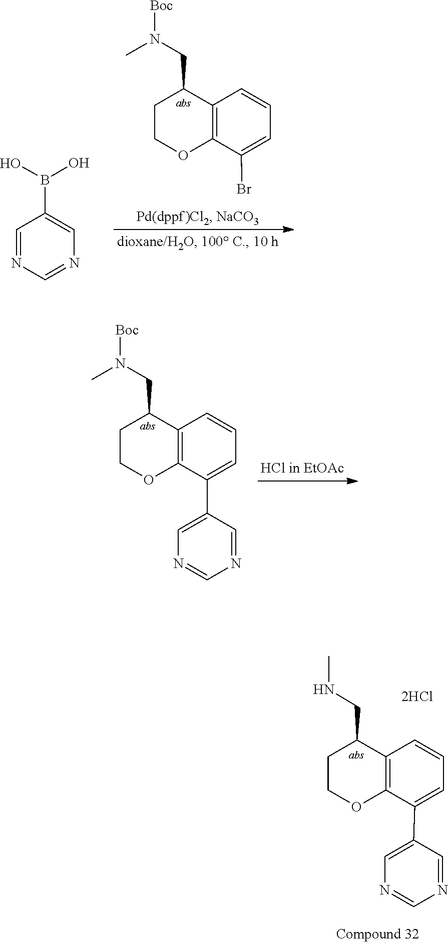

Synthesis of tert-butyl (R)-methyl((8-(pyrimidin-5-yl)chroman-4-yl)methyl)carbamate: A solution of tert-butyl (R)-((8-bromochroman-4-yl)methyl)(methyl)carbamate (200 mg, 0.5843 mmol) and (pyrimidin-5-yl)boronic acid (83.4 mg, 701 μmol) in dioxane (5 mL) and water (1 mL) was added 1,1′-Bis(diphenylphosphino)ferrocene-palladium(II)dichloride dichloromethane complex (92.5 mg, 112 μmol) and Na₂CO₃ (178 mg, 1.68 mmol). The reaction was stirred at 90° C. under argon for 16 h. Water (10 mL) and ethyl acetate (10 mL) was added to the reaction vessel and the resulting biphasic mixture was transferred to a separatory funnel. The layers were separated and the organic phase was triturated with saturated aqueous NaCl (2×15 mL). The combined organics were dried over anhydrous Na₂SO₄, filtered and concentrated in vacuo to afford crude product which was purified by flash column chromatography with a gradient elution of petroleum ether to petrol ether/ethyl acetate=1:1 to provide tert-butyl (R)-methyl((8-(pyrimidin-5-yl)chroman-4-yl)methyl)carbamate (156 mg, yield: 78.3%) as a yellow oil. MS(ESI): m/z=356 [M+H]⁺.

Synthesis of Compound 32: To a solution of tert-butyl (R)-methyl((8-(pyrimidin-5-yl)chroman-4-yl)methyl)carbamate (156 mg, 0.4388 mmol) in ethyl acetate (5 mL) was added HCl in ethyl acetate (10 mL, 3M, 30.0 mmol). The reaction was stirred at ambient temperature for 2 h. The reaction was filtered, and the solid was triturated with ethyl acetate (10 mL) and dried by freeze drying to provide Compound 32 (91.9 mg, 0.3601 mmol) as a white solid. MS(ESI): m/z 256 [M+H]⁺. ¹H NMR (400 MHz, DMSO) δ 9.35 (s, 2H), 9.15 (s, 1H), 8.94 (s, 2H), 7.43 (d, J=7.5 Hz, 1H), 7.34 (d, J=7.5 Hz, 1H), 7.05 (t, J=7.6 Hz, 1H), 6.67 (s, 1H), 4.30-4.10 (m, 2H), 3.38 (s, 1H), 3.31-3.09 (m, 2H), 2.60 (s, 3H), 2.33-2.15 (m, 1H), 2.15-1.96 (m, 1H).

### Example 20: Synthesis of Compound 33

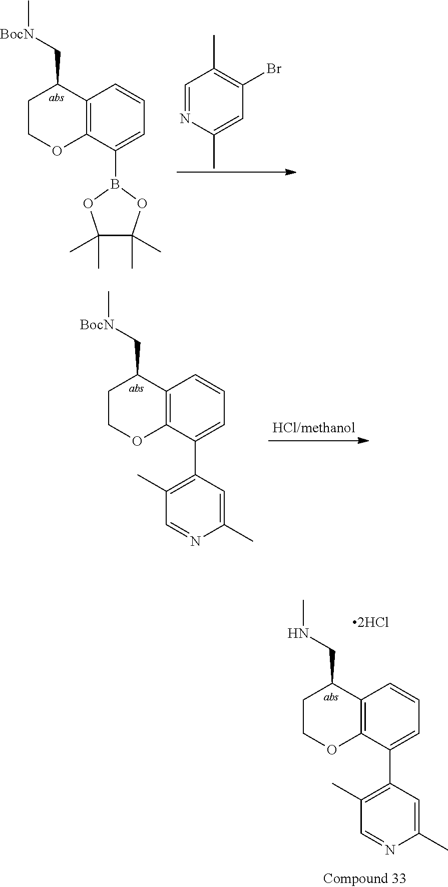

Synthesis of tert-butyl (R)-((8-(2,5-dimethylpyridin-4-yl)chroman-4-yl)methyl)(methyl)carbamate: To a solution of tert-butyl (R)-methyl((8-(4,4,5,5-tetramethyl-1,3,2-dioxaborolan-2-yl)chroman-4-yl)methyl)carbamate (337 mg, 1.05 mmol) and 4-bromo-2,5-dimethylpyridine (226.9 mg, 1.12 mmol) in dioxane (10 mL) was added H₂O (2 mL), 1,1′-Bis(diphenylphosphino)ferrocene-palladium(II)dichloride dichloromethane complex (173 mg, 210 μmol) and Na₂CO₃ (333 mg, 3.15 mmol). The reaction was stirred at 100° C. for 16 h. Upon the completion, EtOAc (30 mL) and water (50 mL) was added and the organic phase was washed with brine (80 mL), dried and concentrated. The resulting oil was purified by flash column chromatography with a gradient elution of petroleum ether (100%) to petroleum ether (30%) and EtOAc (70%) to tert-butyl (R)-((8-(2,5-dimethylpyridin-4-yl)chroman-4-yl)methyl)(methyl)carbamate (127 mg, yield: 40.1%) as a yellow oil. (ESI): m/z=383[M+H]⁺.

Synthesis of Compound 33: To a solution of tert-butyl (R)-((8-(2,5-dimethylpyridin-4-yl)chroman-4-yl)methyl)(methyl)carbamate (127 mg, 332 μmol) in methanol (3 mL) was added HCl/methanol (4M, 10 mL, 40 mmol). The reaction was stirred at ambient temperature for 16 h. Upon the completion, the solvent was removed and the solid was dried by freeze dryer. Compound 33 as an off white solid (56.26 mg, yield: 48%) was obtained. (ESI): m/z=283[M+H]⁺. ¹H NMR (500 MHz, MeOD) δ 8.61 (s, 1H), 7.74 (s, 1H), 7.51 (d, J=7.5 Hz, 1H), 7.19˜7.13 (m, 2H), 4.24 (d, J=6 Hz, 2H), 3.48˜3.35 (m, 3H), 2.84 (s, 3H), 2.79 (s, 3H), 2.32 (s, 3H), 2.22˜2.1203 (m, 2H).

### Example 21: Synthesis of Compound 34, Compound 35

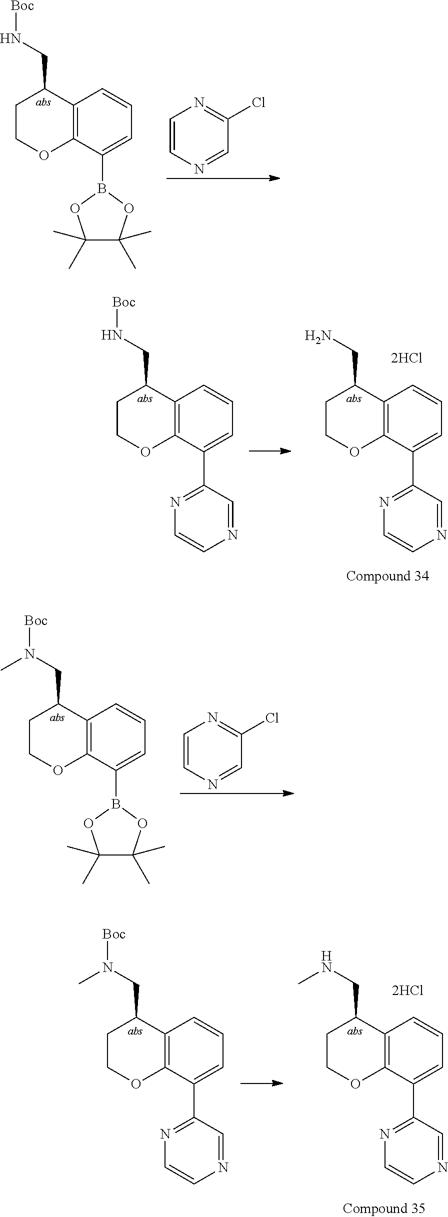

Synthesis of (R)-tert-butyl (8-(pyrazin-2-yl)chroman-4-yl)methylcarbamate: A solution of 2-chloropyrazine (0.06 g, 0.51 mmol, 1.0 equiv.), 1,1′-Bis(diphenylphosphino)ferrocene-palladium(II)dichloride dichloromethane complex (0.056 g, 0.077 mmol, 0.15 equiv.), Na₂CO₃ (0.134 g, 1.27 mmol, 2.5 equiv) and (R)-tert-butyl(8-(4,4,5,5-tetramethyl-1,3,2-dioxaborolan-2-yl)chroman-4-yl)methylcarbamate (0.2 g, 0.51 mmol, 1.0 equiv.) in dioxane/water (20 mL, 4:1) was stirred at 100° C. for 10 h. The reaction mixture was concentrated, water (50 mL) was added, and then extracted with ethyl acetate (100 mL), the organic phase was dried and concentrated to get the crude product which was purified by silica gel column (petrol ether/ethyl acetate=3/1) to give (R)-tert-butyl (8-(pyrazin-2-yl)chroman-4-yl)methylcarbamate (0.1 g, yield: 83%). (ESI): m/z=342[M+H]⁺

Synthesis of Compound 34: To a solution of (R)-tert-butyl (8-(pyrazin-2-yl)chroman-4-yl)methylcarbamate (0.1 g, 0.29 mmol) in HCl/dioxane (20 mL, 4N, 80 mmol) was stirred at rt for 3 h. After concentration, the crude product was triturated with ethyl acetate (10 mL). The solid was collected and dried to get Compound 34 (33 mg, yield: 47%). (ESI): m/z=242 [M+H]⁺. ¹H NMR (400 MHz, CD₃OD) δ 9.13 (s, 1H), 8.77-8.76 (m, 1H), 8.57 (d, J=2.8 Hz, 1H), 7.72-7.69 (m, 1H), 7.42-7.40 (m, 1H), 7.13 (t, J=8.0 Hz, 1H), 4.36-4.32 (m, 2H), 3.50-3.40 (m, 2H), 3.32-3.22 (m, 1H), 2.28-2.24 (m, 1H), 2.11-2.05 (m, 1H).

Synthesis of (R)-tert-butyl methyl((8-(pyrazin-2-yl)chroman-4-yl)methyl)carbamate: A solution of 2-chloropyrazine (0.057 g, 0.5 mmol, 1.0 equiv.), 1,1′-Bis(diphenylphosphino)ferrocene-palladium(II)dichloride dichloromethane complex (0.054 g, 0.05 mmol, 0.15 equiv.), Na₂CO₃ (0.131 g, 1.24 mmol, 2.5 equiv) and (R)-tert-butyl methyl((8-(4,4,5,5-tetramethyl-1,3,2-dioxaborolan-2-yl)chroman-4-yl)methyl)carbamate (0.2 g, 0.50 mmol, 1.0 equiv.) in dioxane/water (20 mL, 4:1) was stirred at 100° C. for 10 h. The reaction mixture was concentrated, water (50 mL) was added, then extracted with ethyl acetate (50 mL), the organic phase was dried and concentrated to get the crude product which was purified by silica gel column (petrol ether/ethyl acetate=3/1) to give (R)-tert-butyl methyl((8-(pyrazin-2-yl)chroman-4-yl)methyl)carbamate (0.07 g, yield: 40%). (ESI): m/z=356[M+H]⁺

Synthesis of Compound 35: To a solution of (R)-tert-butyl methyl((8-(pyrazin-2-yl)chroman-4-yl)methyl)carbamate (0.07 g, 0.20 mmol) in HCl/dioxane (20 mL, 4N) was stirred at rt for 3 h, and then concentrated to get the crude product which was triturated with ethyl acetate (5 mL), the solid was collected and dried to get Compound 35 (15 mg, yield: 23%). (ESI): m/z=256 [M+H]⁺. ¹H NMR (400 MHz, CD₃OD) δ 9.17 (s, 1H), 8.80 (bs, 1H), 8.60 (bs, 1H), 7.74-7.71 (m, 1H), 7.47-7.44 (m, 1H), 7.13 (t, J=8.0 Hz, 1H), 4.37-4.33 (m, 2H), 3.47-3.36 (m, 3H), 2.83 (s, 3H), 2.29-2.27 (m, 1H), 2.14-2.13 (m, 1H).

### Example 22: Synthesis of Compound 36, Compound 37

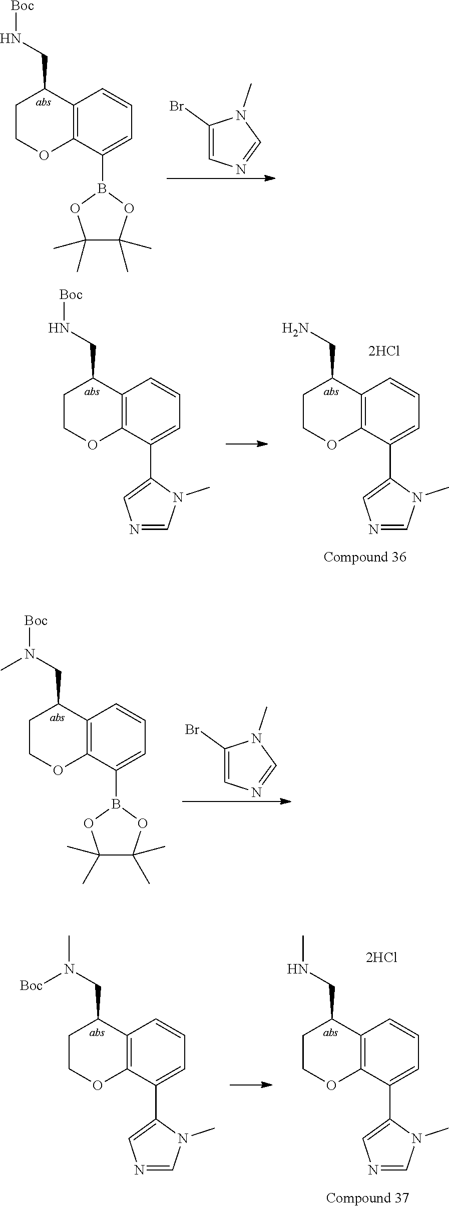

Synthesis of (R)-tert-butyl (8-(1-methyl-1H-imidazol-5-yl)chroman-4-yl)methylcarbamate: A solution of 5-bromo-1-methyl-1H-imidazole (0.08 g, 0.51 mmol, 1.0 equiv.), 1,1′-Bis(diphenylphosphino)ferrocene-palladium(II)dichloride dichloromethane complex (0.056 g, 0.077 mmol, 0.15 equiv.), Na₂CO₃ (0.135 g, 1.29 mmol, 2.5 equiv) and (R)-tert-butyl methyl((8-(4,4,5,5-tetramethyl-1,3,2-dioxaborolan-2-yl)chroman-4-yl)methyl)carbamate (0.2 g, 0.51 mmol, 1.0 equiv.) in dioxane/water (20 mL, 4:1) was stirred at 100° C. for 10 h. The reaction mixture was concentrated, and then water (50 mL) was added, the mixture was extracted with ethyl acetate (50 mL×3), the organic phase was dried and concentrated to get the crude product which was purified by silica gel column (petrol ether/ethyl acetate=3/1) to give (R)-tert-butyl (8-(1-methyl-1H-imidazol-5-yl)chroman-4-yl)methylcarbamate (0.11 g, yield: 62%). (ESI): m/z=344[M+H]⁺

Synthesis of Compound 36: To a solution of (R)-tert-butyl (8-(1-methyl-1H-imidazol-5-yl)chroman-4-yl)methylcarbamate (0.11 g, 0.32 mmol) in HCl/dioxane (20 mL, 4N) was stirred at rt for 3 h. It was concentrated to get the crude product which was triturated with ethyl acetate (5 mL), the solid was collected, and then dried to get Compound 36 (50 mg, yield: 64%). (ESI): m/z=244[M+H]⁺. ¹H NMR (400 MHz, CD₃OD) δ 9.00 (s, 1H), 7.56-7.51 (m, 2H), 7.32-7.29 (m, 1H), 7.12 (t, J=8.0 Hz, 1H), 4.32-4.28 (m, 2H), 3.78 (s, 3H), 3.44-3.41 (m, 1H), 3.35-3.32 (m, 1H), 3.32-3.23 (m, 1H), 2.24-2.20 (m, 1H), 2.10-2.09 (m, 1H).

Synthesis of (R)-tert-butyl methyl((8-(1-methyl-1H-imidazol-5-yl)chroman-4-yl)methyl)carbamate: A solution of 5-bromo-1-methyl-1H-imidazole (0.080 g, 0.5 mmol, 1.0 equiv.), 1,1′-Bis(diphenylphosphino)ferrocene-palladium(II)dichloride dichloromethane complex (0.054 g, 0.074 mmol, 0.15 equiv.), Na₂CO₃ (0.131 g, 1.24 mmol, 2.5 equiv) and (R)-tert-butyl methyl((8-(4,4,5,5-tetramethyl-1,3,2-dioxaborolan-2-yl)chroman-4-yl)methyl)carbamate (0.2 g, 0.50 mmol, 1.0 equiv.) in dioxane/water (20 mL, 4:1) was stirred at 100° C. for 10 h. The reaction mixture was concentrated, and then water (50 mL) was added, the mixture was extracted with ethyl acetate (50 mL×3), the organic phase was dried and concentrated to get the crude product which was purified by silica gel column (petrol ether/ethyl acetate=3/1) to give (R)-tert-butyl methyl((8-(1-methyl-1H-imidazol-5-yl)chroman-4-yl)methyl)carbamate (0.06 g, yield: 34%). (ESI): m/z=358[M+H]⁺

Synthesis of Compound 37: To a solution of (R)-tert-butyl methyl((8-(1-methyl-1H-imidazol-5-yl)chroman-4-yl)methyl)carbamate (0.06 g, 0.17 mmol) in HCl/dioxane (20 mL, 4N) was stirred at rt for 3 h. It was concentrated to get the crude product which was triturated with ethyl acetate (5 mL), the solid was collected, and then dried to get Compound 37 (12 mg, yield: 28%). (ESI): m/z=258 [M+H]⁺. 1H NMR (400 MHz, CD₃OD) δ 9.00 (s, 1H), 7.56-7.53 (m, 2H), 7.32-7.30 (m, 1H), 7.13 (t, J=7.6 Hz, 1H), 4.34-4.27 (m, 2H), 3.78 (s, 3H), 3.44-3.41 (m, 3H), 2.83 (s, 3H), 2.29-2.21 (m, 1H), 2.14-2.07 (m, 1H).

### Example 23: Synthesis of Compound 38, Compound 39

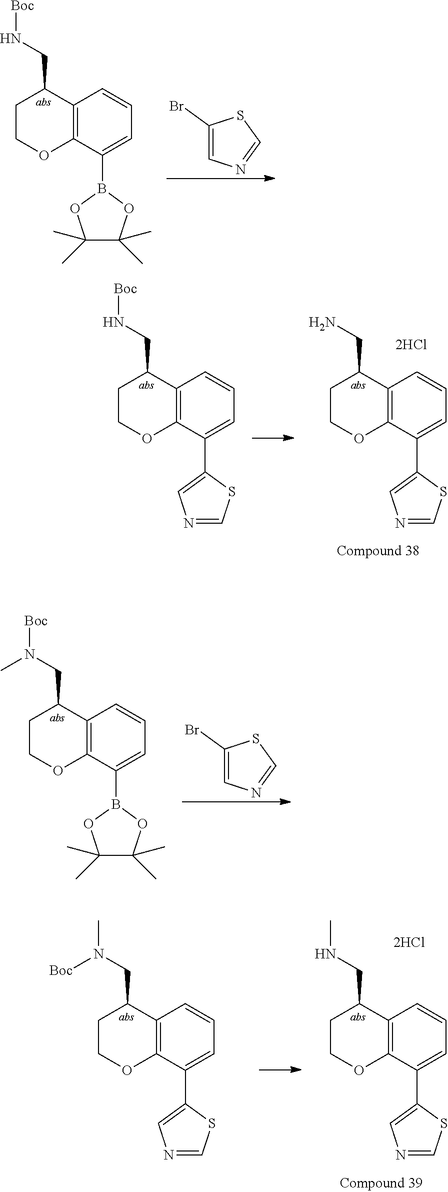

Synthesis of (R)-tert-butyl (8-(thiazol-5-yl)chroman-4-yl)methylcarbamate: A solution of 5-bromo-1,3-thiazole (0.09 g, 0.52 mmol, 1.0 equiv.), 1,1′-Bis(diphenylphosphino)ferrocene-palladium(II)dichloride dichloromethane complex (0.056 g, 0.077 mmol, 0.15 equiv.), Na₂CO₃ (0.136 g, 1.29 mmol, 2.5 equiv) and (R)-tert-butyl (8-(4,4,5,5-tetramethyl-1,3,2-dioxaborolan-2-yl)chroman-4-yl)methylcarbamate (0.2 g, 0.51 mmol, 1.0 equiv.) in dioxane/water (20 mL, 4:1) was stirred at 100° C. for 10 h, monitored by LC-MS. The reaction mixture was concentrated, then water (50 mL) was added, the mixture was extracted with ethyl acetate (50 mL), the organic phase was dried and concentrated to get the crude product which was purified by silica gel column (petrol ether/ethyl acetate=3/1) to give (R)-tert-butyl (8-(thiazol-5-yl)chroman-4-yl)methylcarbamate (0.1 g, yield: 56%). (ESI): m/z=347[M+H]⁺

Synthesis of Compound 38: To a solution of (R)-tert-butyl (8-(thiazol-5-yl)chroman-4-yl)methylcarbamate (0.1 g, 0.29 mmol) in HCl/dioxane (20 mL, 4N) was stirred at rt for 3 h. It was concentrated to get the crude product which was triturated with ethyl acetate (6 mL), the solid was collected, and then dried to get Compound 38 (21 mg, yield: 30%). (ESI): m/z=247 [M+H]⁺. ¹H NMR (400 MHz, CD₃OD) δ 9.86 (bs, 1H), 8.79 (s, 1H), 8.84 (d, J=8.0, 6 Hz, 1H), 7.44 (d, J=7.2 Hz, 1H), 7.14 (t, J=7.6 Hz, 1H), 4.57-4.48 (m, 2H), 3.43-3.36 (m, 2H), 3.27-3.22 (m, 1H), 2.30-2.26 (m, 1H), 2.17-2.13 (m, 1H).

Synthesis of (R)-tert-butyl (8-(thiazol-5-yl)chroman-4-yl)methylcarbamate: A solution of 5-bromo-1,3-thiazole (0.082 g, 0.5 mmol, 1.0 equiv.), 1,1′-Bis(diphenylphosphino)ferrocene-palladium(II)dichloride dichloromethane complex (0.055 g, 0.075 mmol, 0.15 equiv.), Na₂CO₃ (0.131 g, 1.24 mmol, 2.5 equiv) and (R)-tert-butyl methyl((8-(4,4,5,5-tetramethyl-1,3,2-dioxaborolan-2-yl)chroman-4-yl)methyl)carbamate (0.2 g, 0.50 mmol, 1.0 equiv.) in dioxane/water (20 mL, 4:1) was stirred at 100° C. for 10 h, monitored by LC-MS. The reaction mixture was concentrated, and then water (50 mL) was added, the mixture was extracted with ethyl acetate (50 mL×3), the organic phase was dried and concentrated to get the crude product which was purified by silica gel column (petrol ether/ethyl acetate=4/1) to give (R)-tert-butyl (8-(thiazol-5-yl)chroman-4-yl)methylcarbamate (0.07 g, yield: 39%). (ESI): m/z=361[M+H]⁺

Synthesis of Compound 39: To a solution of (R)-tert-butyl (8-(thiazol-5-yl)chroman-4-yl)methylcarbamate (0.07 g, 0.194 mmol) in HCl/dioxane (20 mL, 4N) was stirred at rt for 3 h. The mixture was concentrated, the solid was collected and triturated with ethyl acetate (5 mL) to give Compound 39 (30 mg, yield: 30%). (ESI): m/z=261 [M+H]⁺. 1H NMR (400 MHz, CD₃OD) δ 9.66 (bs, 1H), 8.67 (s, 1H), 7.79-7.77 (m, 1H), 7.45 (d, J=7.2 Hz, 1H), 7.10 (t, J=7.6 Hz, 1H), 4.53-4.43 (m, 2H), 3.48-3.35 (m, 3H), 2.83 (s, 3H), 2.29-2.20 (m, 2H).

### Example 24: Synthesis of Compound 40

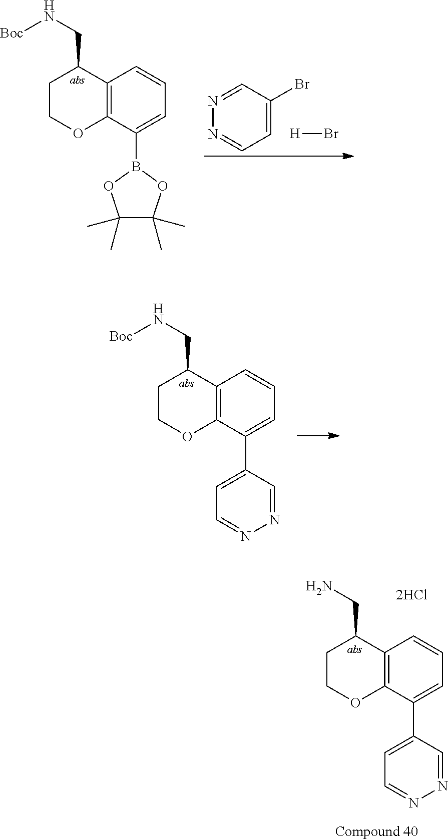

Synthesis of tert-butyl (R)-((8-(pyridazin-4-yl)chroman-4-yl)methyl)carbamate: A solution of tert-butyl (R)-((8-(4,4,5,5-tetramethyl-1,3,2-dioxaborolan-2-yl)chroman-4-yl)methyl)carbamate (200 mg, 0.5137 mmol) and 4-bromopyridazine hydrobromide (147 mg, 616 μmol) in dioxane (5 mL) and water (1 mL) was added 1,1′-Bis(diphenylphosphino)ferrocene-palladium(II)dichloride dichloromethane complex (83.2 mg, 102 μmol) and sodium carbonate (271 mg, 2.56 mmol). The reaction was stirred at 90° C. under argon for 16 h. Water (10 mL) and ethyl acetate (10 mL) was added to the reaction vessel and the resulting biphasic mixture was transferred to a separatory funnel. The layers were separated and the organic phase was triturated with saturated aqueous NaCl (2×15 mL). The combined organic layers were dried over anhydrous Na₂SO₄, filtered and concentrated in vacuo to afford crude product. The crude product was purified by flash column chromatography with a gradient elution of petroleum ether to petrol ether/ethyl acetate=1:1 to provide tert-butyl (R)-((8-(pyridazin-4-yl)chroman-4-yl)methyl)carbamate (80.0 mg, 0.2343 mmol, yield: 45.7%) as a yellow oil. MS(ESI): m/z 342 [M+H]⁺.

Synthesis of Compound 40: To a solution of tert-butyl (R)-((8-(pyridazin-4-yl)chroman-4-yl)methyl)carbamate (80 mg, 0.2343 mmol) in ethyl acetate (2 mL) was added HCl in ethyl acetate (5 mL, 3M, 15.0 mmol). The reaction was stirred at ambient temperature for 2 h. The reaction was filtered and the solid was triturated with ethyl acetate (10 mL). The solid was dissolved in water and dried by lyophilization to provide Compound 40 (54.6 mg, 0.2265 mmol) as a white solid. MS(ESI): m/z 242 [M+H]⁺. ¹H NMR (400 MHz, CD₃OD) δ 9.87 (s, 1H), 9.56 (d, J=5.8 Hz, 1H), 8.74 (s, 1H), 7.67 (d, J=7.8 Hz, 1H), 7.59 (d, J=7.7 Hz, 1H), 7.23 (t, J=7.7 Hz, 1H), 4.54-4.24 (m, 2H), 3.54-3.36 (m, 2H), 3.29-3.20 (m, 1H), 2.38-2.21 (m, 1H), 2.21-2.05 (m, 1H).

### Example 25: Synthesis of Compound 41

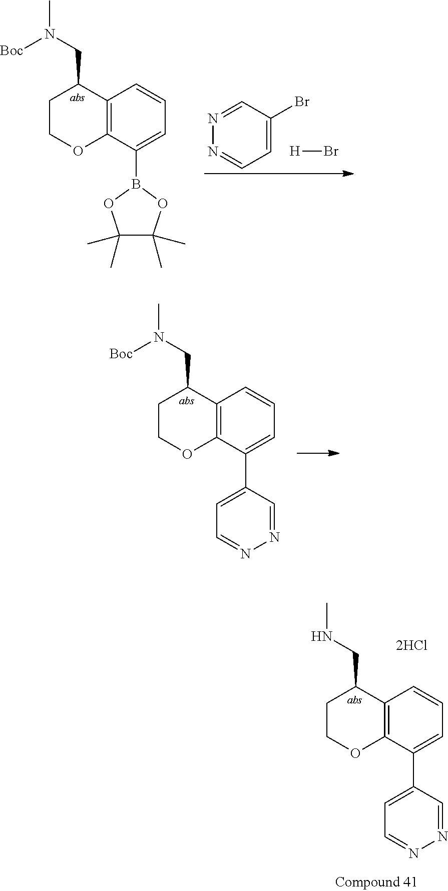

Synthesis of tert-butyl (R)-methyl((8-(pyridazin-4-yl)chroman-4-yl)methyl)carbamate: A solution of tert-butyl (R)-methyl((8-(4,4,5,5-tetramethyl-1,3,2-dioxaborolan-2-yl)chroman-4-yl)methyl)carbamate (200 mg, 0.5137 mmol) and 4-bromopyridazine hydrobromide (142 mg, 594 μmol) in dioxane (5 mL) and water (1 mL) was added 1,1′-Bis(diphenylphosphino)ferrocene-palladium(II)dichloride dichloromethane complex (80.9 mg, 99.1 μmol) and sodium carbonate (261 mg, 2.47 mmol). The reaction was stirred at 90° C. under argon for 16 h. Water (10 mL) and ethyl acetate (10 mL) was added to the reaction vessel and the resulting biphasic mixture was transferred to a separatory funnel. The layers were separated and the organic phase was washed with saturated aqueous NaCl (2×15 mL). The combined organics were dried over anhydrous Na₂SO₄, filtered and concentrated in vacuo to afford crude product. The crude product was purified by flash column chromatography with a gradient elution of petroleum ether to petrol ether/ethyl acetate=1:1 to provide tert-butyl (R)-methyl((8-(pyridazin-4-yl)chroman-4-yl)methyl)carbamate (81.1 mg, 0.2283 mmol, yield: 46.3%) as a yellow oil. MS(ESI): m/z 356 [M+H]⁺.

Synthesis of Compound 41: To a solution of tert-butyl (R)-methyl((8-(pyridazin-4-yl)chroman-4-yl)methyl)carbamate (82 mg, 0.2307 mmol) in ethyl acetate (2 mL) was added HCl in ethyl acetate (1.53 mL, 3N, 4.61 mmol). The reaction was stirred at ambient temperature for 2 h. The reaction was filtered and the solid was triturated with ethyl acetate (10 mL). The solid was dissolved in water and then dried by freeze dryer to provide Compound 41 (46.1 mg, 0.1807 mmol) as a white solid. MS(ESI): m/z 256 [M+H]⁺. ¹H NMR (400 MHz, CD₃OD) δ 9.89 (s, 1H), 9.57 (d, J=5.7 Hz, 1H), 8.76 (s, 1H), 7.68 (d, J=7.6 Hz, 1H), 7.62 (d, J=7.6 Hz, 1H), 7.23 (t, J=7.7 Hz, 1H), 4.45-4.39 (m, 2H), 3.49-3.3 (m, 3H), 2.84 (s, 3H), 2.37-2.24 (m, 1H), 2.24-2.12 (m, 1H).

### Example 26: Synthesis of Compound 42

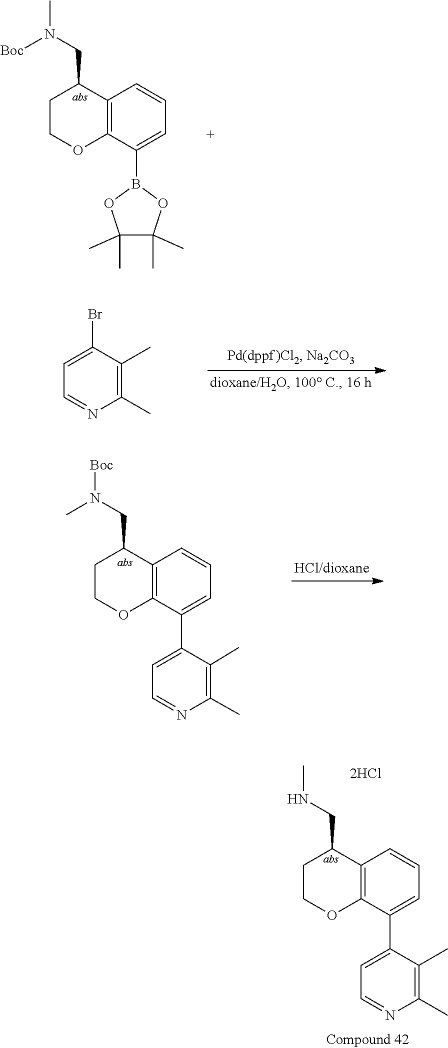

Synthesis of tert-butyl (R)-((8-(2,3-dimethylpyridin-4-yl)chroman-4-yl)methyl)carbamate: To a solution of methyl({[(4R)-8-(4,4,5,5-tetramethyl-1,3,2-dioxaborolan-2-yl)-3,4-dihydro-2H-1-benzopyran-4-yl]methyl})amine (337 mg, 1.05 mmol) and 4-bromo-2,5-dimethylpyridine (208 mg, 1.12 mmol) in dioxane (10 mL), then water (2 mL), 1,1′-Bis(diphenylphosphino)ferrocene-palladium(II)dichloride dichloromethane complex (173 mg, 210 μmol) and Na₂CO₃ (333 mg, 3.15 mmol) were added. The reaction was stirred at 100° C. for 16 h. Water (10 mL) was added to the reaction vessel and the resulting biphasic mixture was transferred to a separatory funnel. The layers were separated and the organic phase was washed with water (3×15 mL) and brine (2×10 mL). The combined organic layers were dried over anhydrous Na₂SO₄, filtered and concentrated in vacuum to afford crude product which was purified by flash column chromatography with an isocratic elution of petroleum ether (30%) and ethyl acetate (70%) to provide [(4R)-8-(2,5-dimethylpyridin-4-yl)-3,4-dihydro-2H-1-benzopyran-4-yl]methyl}(methyl)amine (148 mg, 527 μmol, yield: 50.0%) as a yellow oil. MS (ESI): m/z=382.9[M+H]⁺.

Synthesis of [(4R)-8-(2,3-dimethylpyridin-4-yl)-3,4-dihydro-2H-1-benzopyran-4-yl]methyl}(methyl)amine hydrochloride salt: To a solution of tert-butyl N-{[(4R)-8-(2,3-dimethylpyridin-4-yl)-3,4-dihydro-2H-1-benzopyran-4-yl]methyl}-N-methylcarbamate (152 mg, 397 μmol) in methanol (3 mL) was added 4N HCl/dioxane (2 mL, 8 mmol). The reaction was stirred at ambient temperature for 16 h. The mixture was concentrated to provide [(4R)-8-(2,3-dimethylpyridin-4-yl)-3,4-dihydro-2H-1-benzopyran-4-yl]methyl}(methyl)amine hydrochloride salt (Compound 42) as a yellow solid. (77.7 mg, yield: 69.3%). MS (ESI): m/z=282.9[M+H]⁺. ¹H NMR (400 MHz, CD₃OD) δ 8.52 (d, J=5.9 Hz, 1H), 7.70 (d, J=5.8 Hz, 1H), 7.49 (d, J=6.9 Hz, 1H), 7.21-7.05 (m, 2H), 4.21 (s, 2H), 3.42 (t, J=22.3 Hz, 3H), 2.81 (d, J=8.2 Hz, 6H), 2.28 (s, 3H), 2.19 (s, 1H), 2.08 (d, J=12.4 Hz, 1H).

### Example 27: Synthesis of Compound 43

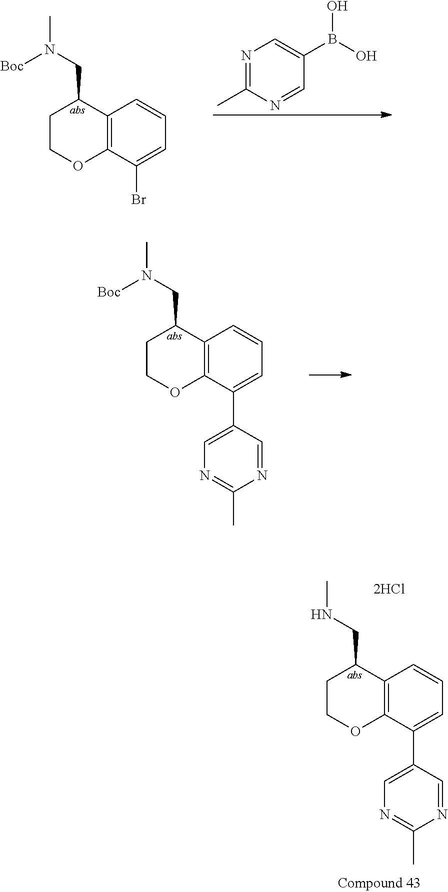

Synthesis of tert-butyl (R)-methyl((8-(2-methylpyrimidin-5-yl)chroman-4-yl)methyl)carbamate: A solution of tert-butyl (R)-((8-bromochroman-4-yl)methyl)(methyl)carbamate (200 mg, 0.5613 mmol) and (2-methylpyrimidin-5-yl)boronic acid (92.8 mg, 673 μmol) in dioxane (5 mL) and water (1 mL) was added 1,1′-Bis(diphenylphosphino)ferrocene-palladium(II)dichloride dichloromethane complex (45.8 mg, 56.1 μmol) and Na₂CO₃ (178 mg, 1.68 mmol). The reaction was stirred at 90° C. under argon for 16 h. Water (10 mL) and ethyl acetate (10 mL) was added to the reaction vessel and the resulting biphasic mixture was transferred to a separatory funnel. The layers were separated and the organic phase was triturated with saturated aqueous NaCl (2×15 mL). The combined organics were dried over anhydrous Na₂SO₄, filtered and concentrated in vacuo to afford crude product. The crude product was purified by flash column chromatography with a gradient elution of petroleum ether to petrol ether/ethyl acetate=1:1 to provide tert-butyl (R)-methyl((8-(2-methylpyrimidin-5-yl)chroman-4-yl)methyl)carbamate (148 mg, 0.4019 mmol, yield: 71.4%) as a yellow oil. MS(ESI): m/z 370 [M+H]⁺.

Synthesis of Compound 43: To a solution of tert-butyl (R)-methyl((8-(2-methylpyrimidin-5-yl)chroman-4-yl)methyl)carbamate (150 mg, 0.4059 mmol) in ethyl acetate (5 mL) was added HCl in ethyl acetate (10 mL, 3M, 30.0 mmol). The reaction was stirred at ambient temperature for 3 h. The reaction was filtered and the solid was triturated with ethyl acetate (10 mL) and freeze drying on freeze dryer to provide Compound 43 (25.9 mg, 0.09645 mmol) as a white solid. MS(ESI): m/z 270 [M+H]⁺. HCl salt ¹H NMR (400 MHz, CD₃OD) δ 9.24 (s, 2H), 7.38 (d, J=7.8 Hz, 2H), 7.05 (t, J=7.7 Hz, 1H), 4.32-4.12 (m, 2H), 3.38-3.23 (m, 3H), 2.84 (s, 3H), 2.72 (s, 3H), 2.23-2.07 (m, 1H), 2.07-1.96 (m, 1H). Free base: ¹H NMR (400 MHz, CD₃OD) δ 8.80 (s, 2H), 7.32-7.26 (m, 1H), 7.24-7.19 (m, 1H), 7.00 (t, J=7.6 Hz, 1H), 4.26-4.14 (m, 2H), 3.15-3.03 (m, 1H), 2.92 (dd, J=12.2, 4.6 Hz, 1H), 2.86-2.76 (m, 1H), 2.71 (s, 3H), 2.46 (s, 3H), 2.16-1.98 (m, 2H).

### Example 28: Synthesis of Compound 44

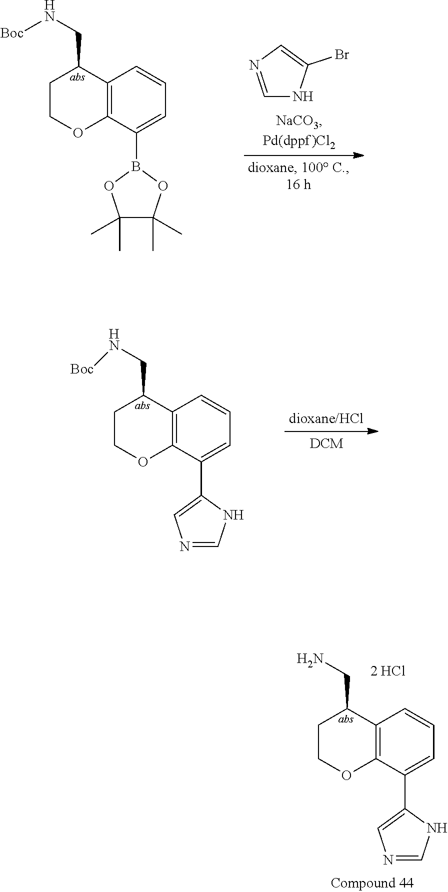 [SMILES: CC(C)(C)OC(=O)NC[C@@H]1CCOc2c(B3OC(C)(C)C(C)(C)O3)cccc21 ; Brc1cnc[nH]1 ; CC(C)(C)OC(=O)NC[C@@H]1CCOc2c(-c3cnc[nH]3)cccc21 ; NC[C@@H]1CCOc2c(-c3cnc[nH]3)cccc21]

Synthesis of tert-butyl (R)-((8-(1H-imidazol-5-yl)chroman-4-yl)methyl)carbamate: To a solution of tert-butyl N-{[(4R)-8-(4,4,5,5-tetramethyl-1,3,2-dioxaborolan-2-yl)-3,4-dihydro-2H-1-benzopyran-4-yl]methyl}carbamate (200 mg, 0.513 mmol, 1.0 eq) and 5-bromo-1H-imidazole (113 mg, 0.769 mmol, 1.5 eq) in dioxane (20 mL) and water (4 ml), was added 1,1′-Bis(diphenylphosphino)ferrocene-palladium(II)dichloride dichloromethane complex (117 mg, 0.102 mmol, 0.2 eq), K₂CO₃ (211 mg, 1.53 mmol, 3.0 eq), then the mixture is stirred at 100° C. for 16 h. Water (10 mL) and ethyl acetate (10 mL) was added to the reaction vessel and the resulting biphasic mixture was transferred to a separatory funnel. The layers were separated and the organic phase was triturated with saturated aqueous NaCl (2×15 mL). The combined organics were dried over anhydrous Na₂SO₄, filtered and concentrated in vacuo to afford crude product which was purified by silica gel column (dichloromethane/methanol=10/1) to give the product (100 mg, purity 95%). ESI-MS: ESI m/z: 330.1[M+H]⁺

Synthesis of Compound 44: To a solution of tert-butyl N-{[(4R)-8-(1H-imidazol-5-yl)-3,4-dihydro-2H-1-benzopyran-4-yl]methyl}carbamate (60 mg, 0.182 mmol, 1.0 eq) in dichloromethane (8 mL) was added HCl/1,4-dioxane (8 ml, 4 M) then the mixture is stirred at RT for 4 h. The reaction was filtered and the solid was washed with ethyl acetate (10 mL) and freeze drying on freeze dryer to provide 1-[(4R)-8-(1H-imidazol-5-yl)-3,4-dihydro-2H-1-benzopyran-4-yl]methanamine (Compound 44) (45.31 mg, purity 100%, yield 108%) as a white solid. MS(ESI): m/z 230 [M+H]⁺. ¹H NMR (400 MHz, CD₃OD) δ 8.99 (d, J=1.2 Hz, 1H), 7.94 (d, J=1.2 Hz, 1H), 7.63 (dd, J=1.2, 8.0 Hz, 1H), 7.41 (dd, J=0.4, 7.6 Hz, 1H), 7.12 (t, J=7.8 Hz, 1H), 4.50-4.40 (m, 2H), 3.43-3.35 (m, 2H), 3.26-3.21 (m, 1H), 2.28-2.24 (m, 1H), 2.15-2.14 (m, 1H).

### Example 29: Synthesis of Compound 45

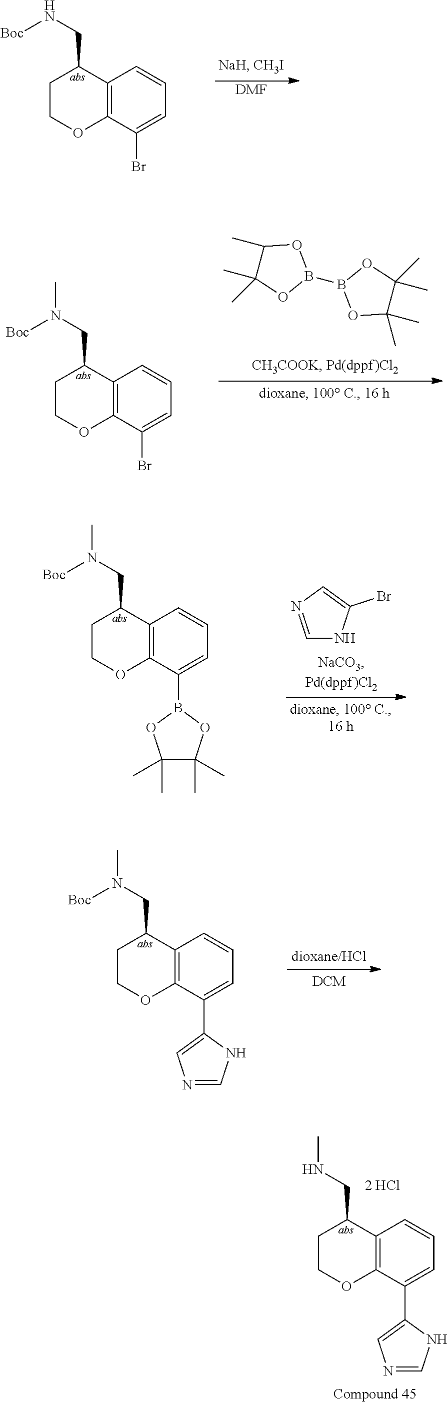

Synthesis of tert-butyl (R)-((8-bromochroman-4-yl)methyl)(methyl)carbamate: To a solution of tert-butyl N-{[(4R)-8-bromo-3,4-dihydro-2H-1-benzopyran-4-yl]methyl}carbamate (8.0 g, 23.3 mmol, 1.0 eq) in N,N-dimethylformamide (80 mL) was added NaH (2.8 g, 69.9 mmol, 3.0 eq) then the mixture was stirred at RT for 30 mins, and then CH₃I (414 mg, 2.92 mmol, 5.0 eq) was added, then the mixture is stirred at RT for 3 h. Water (100 mL) and ethyl acetate (100 mL) was added to the reaction vessel and the resulting biphasic mixture was transferred to a separatory funnel. The layers were separated and the organic phase was triturated with saturated aqueous NaCl (2×150 mL). The combined organics were dried over anhydrous Na₂SO₄, filtered and concentrated in vacuo to afford crude product which was purified by silica gel column (petrol ether/ethyl acetate=10/1) to give the product (7.6 g, 89.4%). ESI-MS: m/z=378.0[M+Na]⁺

Synthesis of tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate: To a solution of tert-butyl N-{[(4R)-8-bromo-3,4-dihydro-2H-1-benzopyran-4-yl]methyl}-N-methylcarbamate (4.5 g, 12.6 mmol, 1.0 eq) and 4,4,5,5-tetramethyl-2-(4,4,5,5-tetramethyl-1,3,2-dioxaborolan-2-yl)-1,3,2-dioxaborolane (4.79 g, 18.9 mmol, 1.5 eq) in 1,4-dioxane (150 mL) was added 1,1′-Bis(diphenylphosphino)ferrocene-palladium(II)dichloride dichloromethane complex (514 mg, 0.630 mmol, 0.05 eq) and potassium acetate (3.70 g, 37.8 mmol, 3.0 eq) then the mixture was stirred at 100° C. for overnight. Water (100 mL) and ethyl acetate (100 mL) was added to the reaction vessel and the resulting biphasic mixture was transferred to a separatory funnel. The layers were separated and the organic phase was triturated with saturated aqueous NaCl (2×150 mL). The combined organics were dried over anhydrous Na₂SO₄, filtered and concentrated in vacuo to afford crude product. The crude product was purified by silica gel column (petrol ether/ethyl acetate=5/1) to get the desired product tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate (5.1 g, yield 100%, purity 95%). MS(ESI): m/z 426 [M+Na]⁺.

Synthesis of tert-butyl N-{[(4R)-8-(1H-imidazol-5-yl)-3,4-dihydro-2H-1-benzopyran-4-yl]methyl}-N-methylcarbamate: To a solution of tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate (300 mg, 0.743 mmol, 1.0 eq) and 5-bromo-1H-imidazole (163 mg, 1.11 mmol, 1.5 eq) in dioxane (30 mL) and water (6 ml), was added 1,1′-Bis(diphenylphosphino)ferrocene-palladium(II)dichloride dichloromethane complex (171 mg, 0.148 mmol, 0.2 eq), potassium carbonate (306 mg, 2.2 mmol, 3.0 eq), then the mixture is stirred at 100° C. for 16 h. The mixture was concentrated and then purified by reverse phase to give tert-butyl N-{[(4R)-8-(1H-imidazol-5-yl)-3,4-dihydro-2H-1-benzopyran-4-yl]methyl}-N-methylcarbamate (50 mg, yield 19.6%). MS(ESI): m/z 344 [M+H]⁺.

Synthesis of Compound 45: To a solution of tert-butyl N-{[(4R)-8-(1H-imidazol-5-yl)-3,4-dihydro-2H-1-benzopyran-4-yl]methyl}-N-methylcarbamate (50 mg, 0.145 mmol, 1.0 eq) in dichloromethane (5 mL) was added HCl/1,4-dioxane (5 ml, 4 M) then the mixture is stirred at room temperature for 4 h. The mixture was concentrated in vacuum, then dissolved in water (3 mL) and then dried on freeze-dryer to obtain {[(4R)-8-(1H-imidazol-5-yl)-3,4-dihydro-2H-1-benzopyran-4-yl]methyl}(methyl)amine hydrochloride as a white solid. (16.05 mg, purity 98.80%, yield 45.4%). MS(ESI): m/z 244 [M+H]⁺. ¹H NMR (400 MHz, CD₃OD) δ 8.99 (d, J=1.2 Hz, 1H), 7.94 (d, J=1.2 Hz, 1H), 7.64 (dd, J=1.2, 7.6 Hz, 1H), 7.43 (dd, J=1.2, 7.6 Hz, 1H), 7.12 (t, J=7.8 Hz, 1H), 4.51-4.41 (m, 2H), 3.47-3.34 (m, 3H), 2.83 (s, 3H), 2.29-2.27 (m, 1H), 2.17-2.16 (m, 1H).

### Example 30: Synthesis of Compound 46

### Synthesis of tert-butyl N-methyl-N-{[(4R)-8-(2-methylpyrimidin-4-yl)-3,4-dihydro-2H-1-benzopyran-4-yl]methyl}carbamate

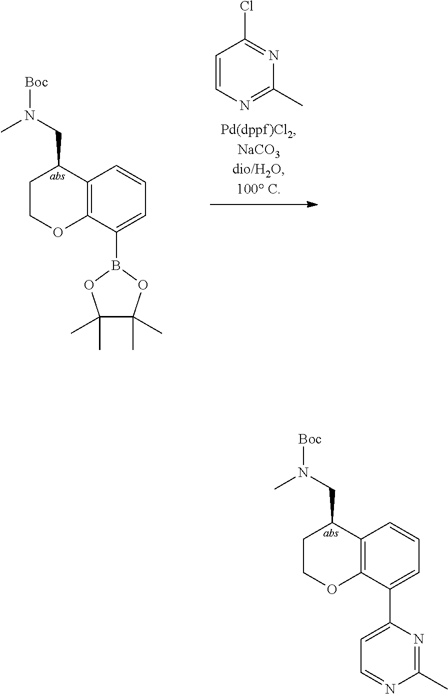

To a solution of tert-butyl N-methyl-N-{[(4R)-8-(4,4,5,5-tetramethyl-1,3,2-dioxaborolan-2-yl)-3,4-dihydro-2H-1-benzopyran-4-yl]methyl}carbamate (0.2 g, 0.495 mmol) in dioxane (10 mL) was added 1,1′-Bis(diphenylphosphino)ferrocene-palladium(II)dichloride dichloromethane complex (36.3 mg, 0.0495 mmol) and Na₂CO₃ (156 mg, 1.48 mmol), 4-chloro-2-methylpyrimidine (63.6 mg, 0.495 mmol) The reaction mixture was heated to 100° C. and stirred at that temperature for 24 h under nitrogen atmosphere. Ethyl acetate (30 mL) was added to the reaction vessel and the resulting biphasic mixture was transferred to a separatory funnel. The layers were separated and the organic phase was washed with saturated aqueous NaCl (4×30 mL). The combined organics were dried over anhydrous Na₂SO₄, filtered and concentrated in vacuo. The resulting solid was purified by flash column chromatography with a gradient elution of petroleum ether (100%) to petroleum ether (60%) and Ethyl acetate (40%) to provide tert-butyl (R)-methyl((8-(2-methylpyrimidin-4-yl)chroman-4-yl)methyl)carbamate (58.9 mg, yield: 32.3%) as a yellow oil. MS(ESI): m/z 370 [M+H]⁺.

### Synthesis of (R)—N-methyl-1-(8-(2-methylpyrimidin-4-yl)chroman-4-yl)methanamine (Compound 46)

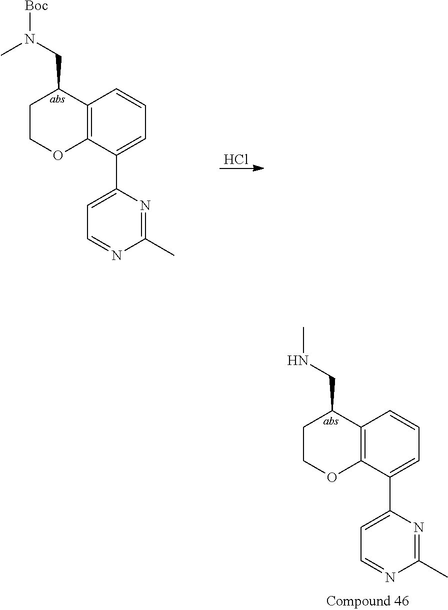

To a solution of tert-butyl (R)-methyl((8-(2-methylpyrimidin-4-yl)chroman-4-yl)methyl)carbamate (90 mg, 0.197 mmol) was added HCl/ethyl acetate (10 ml) The reaction was stirred at room temperature for 2 h. Upon the completion, the mixture was evaporated in vacuo to dryness and then dried on freeze-dryer to yield Compound 46 (35.6 mg yield: 81.6%) as white solid. MS(ESI): m/z 270 [M+H]⁺. 1H NMR (500 MHz, CD₃OD) δ 8.84 (d, J=6.1 Hz, 1H), 8.35 (d, J=6.1 Hz, 1H), 8.23-7.91 (m, 1H), 7.53 (t, J=21.8 Hz, 1H), 7.15 (t, J=7.7 Hz, 1H), 4.41 (ddd, J=21.2, 15.1, 8.1 Hz, 2H), 3.50-3.33 (m, 3H), 2.85 (d, J=32.5 Hz, 6H), 2.32-2.13 (m, 2H).

### Example 31: Synthesis of Compound 47

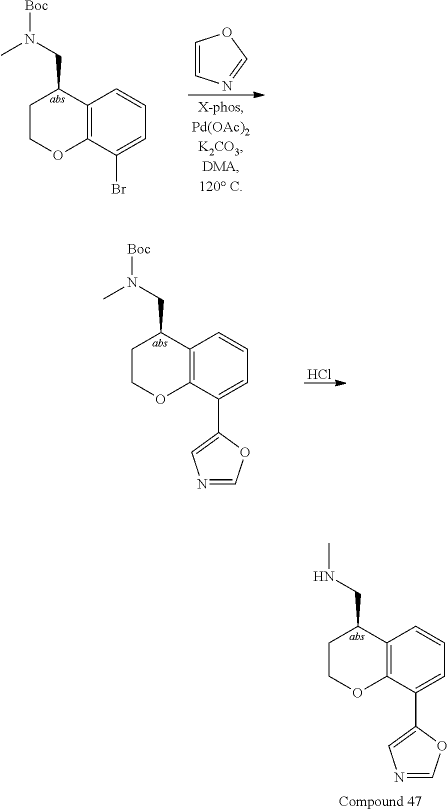 [SMILES: CN(C[C@@H]1CCOc2c(Br)cccc21)C(=O)OC(C)(C)C ; c1cocn1 ; CN(C[C@@H]1CCOc2c(-c3cnco3)cccc21)C(=O)OC(C)(C)C ; Cl ; CNC[C@@H]1CCOc2c(-c3cnco3)cccc21]

Synthesis of tert-butyl N-methyl-N-{[(4R)-8-(3-methylpyridin-4-yl)-3,4-dihydro-2H-1-benzopyran-4-yl]methyl}carbamate: To a solution of tert-butyl N-methyl-N-{[(4R)-8-(1,3-oxazol-5-yl)-3,4-dihydro-2H-1-benzopyran-4-yl]methyl}carbamate (0.2 g, 0.561 mmol) in N,N-dimethylformamide (10 mL) was added Pd(OAc)₂ (12.5 mg, 0.0561 mmol) and K₂CO₃ (231 mg, 1.68 mmol) and X-phos (26.7 mg, 0.0561 mmol), (3-methylpyridin-4-yl)boronic acid (58 mg, 0.841 mmol). The reaction mixture was heated to 120° C. and stirred at that temperature for 24 h under nitrogen atmosphere. Ethyl acetate (30 mL) was added to the reaction vessel and the resulting biphasic mixture was transferred to a separatory funnel. The layers were separated and the organic phase was triturated with saturated aqueous NaCl (4×30 mL). The combined organics were dried over anhydrous Na₂SO₄, filtered and concentrated in vacuo. The resulting solid was purified by flash column chromatography with a gradient elution of petroleum ether (100%) to petroleum ether (70%) and ethyl acetate (30%) to provide tert-butyl N-methyl-N-{[(4R)-8-(3-methylpyridin-4-yl)-3,4-dihydro-2H-1-benzopyran-4-yl]methyl}carbamate (14.7 mg, yield: 7.62%) as a yellow oil. MS(ESI): m/z 345 [M+H]⁺.

Synthesis of methyl({[(4R)-8-(1,3-oxazol-5-yl)-3,4-dihydro-2H-1-benzopyran-4-yl]methyl})amine (Compound 47): To a solution of tert-butyl methyl({[(4R)-8-(1,3-oxazol-5-yl)-3,4-dihydro-2H-1-benzopyran-4-yl]methyl})amine (40 mg, 0.116 mmol) was added HCl/ethyl acetate (10 ml) The reaction was stirred at room temperature for 2 h. Upon the completion, the mixture was evaporated in vacuo to dryness and then freeze-dried to yield Compound 47 (28 mg yield: 98.9%) as a white solid. (ESI) m/z: 245 [M+H]⁺. ¹H NMR (400 MHz, CD₃OD) δ 8.35 (s, 1H), 7.72 (dd, J=7.8, 1.5 Hz, 1H), 7.58 (s, 1H), 7.28 (d, J=6.5 Hz, 1H), 7.06 (t, J=7.7 Hz, 1H), 4.50-4.41 (m, 2H), 3.44-3.38 (m, 3H) 2.80 (s, 3H), 2.28-2.11 (m, 2H).

### Example 32: Synthesis of Compound 48, Compound 49

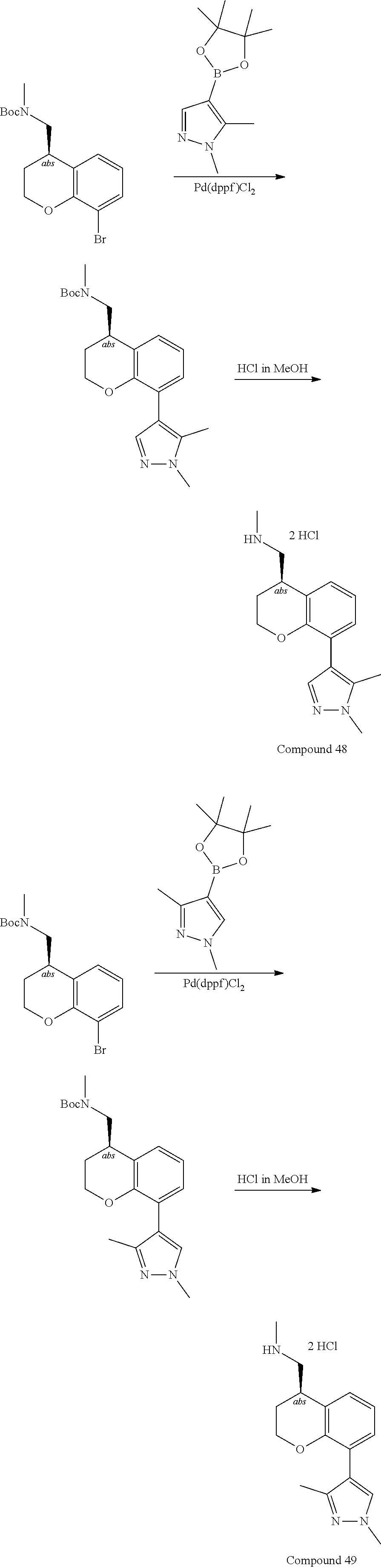

Synthesis of tert-butyl (R)-((8-(1,5-dimethyl-1H-pyrazol-4-yl)chroman-4-yl)methyl)(methyl)carbamate: To a solution of tert-butyl (R)-((8-bromochroman-4-yl)methyl)(methyl)carbamate (100 mg, 0.281 mmol) in dioxane (1 mL) and water (0.25 mL) was added [1,1′-bis(diphenylphosphino)ferrocene]dichloropalladium(II) (20.4 mg, 28.0 μmol), potassium carbonate (116 mg, 841 μmol) and 1,5-dimethyl-4-(4,4,5,5-tetramethyl-1,3,2-dioxaborolan-2-yl)-1H-pyrazole (74.6 mg, 336 μmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-((8-(1,5-dimethyl-1H-pyrazol-4-yl)chroman-4-yl)methyl)(methyl)carbamate (43.5 mg, yield: 42%) as a yellow oil. MS (ESI) m/z: 372 [M+H]⁺.

Synthesis of Compound 48: A solution of tert-butyl (R)-((8-(1,5-dimethyl-1H-pyrazol-4-yl)chroman-4-yl)methyl)(methyl)carbamate (50 mg, 0.135 mmol) in HCl in methanol (4 M, 1 mL) was stirred at ambient temperature for 16 h. The reaction was concentrated in vacuum and the residue was triturated with ethyl acetate (10 mL) and the obtained solid was dried by lyophilization to afford Compound 48 (24.2 mg, yield: 52%) as a white solid. (ESI) m/z: 272[M+H]⁺. ¹H NMR (500 MHz, DMSO) δ 9.12 (s, 2H), 7.43 (s, 1H), 7.22 (d, J=7.5 Hz, 1H), 7.04 (d, J=6.4 Hz, 1H), 6.92 (t, J=7.6 Hz, 1H), 4.24-4.05 (m, 2H), 3.77 (s, 3H), 3.36-3.26 (m, 1H), 3.26-3.14 (m, 2H), 2.60 (t, J=5.2 Hz, 3H), 2.18 (s, 3H), 2.18-2.12 (m, 1H), 2.07-1.97 (m, 1H).

Synthesis of tert-butyl (R)-((8-(1,3-dimethyl-1H-pyrazol-4-yl)chroman-4-yl)methyl)(methyl)carbamate: To a solution of tert-butyl (R)-((8-bromochroman-4-yl)methyl)(methyl)carbamate (100 mg, 0.281 mmol) in dioxane (1 mL) and water (0.25 mL) was added [1,1′-bis(diphenylphosphino)ferrocene]dichloropalladium(II) (20.4 mg, 28.0 μmol), potassium carbonate (116 mg, 841 μmol) and 1,3-dimethyl-4-(4,4,5,5-tetramethyl-1,3,2-dioxaborolan-2-yl)-1H-pyrazole (74.6 mg, 336 μmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-((8-(1,3-dimethyl-1H-pyrazol-4-yl)chroman-4-yl)methyl)(methyl)carbamate (67.9 mg, yield: 65%) as a yellow oil. MS (ESI) m/z: 372 [M+H]⁺.

Synthesis of Compound 49: A solution of tert-butyl (R)-((8-(1,3-dimethyl-1H-pyrazol-4-yl)chroman-4-yl)methyl)(methyl)carbamate (70 mg, 0.188 mmol) in HCl in methanol (4 M, 2 mL) was stirred at ambient temperature for 16 h. The reaction was concentrated in vacuum and the residue was triturated with ethyl acetate (10 mL) and the obtained solid was dried by lyophilization to afford Compound 49 (22.4 mg, yield: 35%) as a white solid. (ESI) m/z: 272[M+H]⁺. ¹H NMR (500 MHz, DMSO) δ 9.16 (s, 2H), 7.74 (s, 1H), 7.21 (d, J=7.6 Hz, 1H), 7.10 (d, J=7.4 Hz, 1H), 6.91 (t, J=7.6 Hz, 1H), 4.23-4.16 (m, 1H), 4.12 (t, J=9.9 Hz, 1H), 3.35-3.27 (m, 1H), 3.27-3.12 (m, 2H), 2.60 (t, J=5.2 Hz, 3H), 2.19-2.15 (m, 1H), 2.14 (s, 3H), 2.07-1.97 (m, 1H).

### Example 33: Synthesis of Compound 50, Compound 51, Compound 52

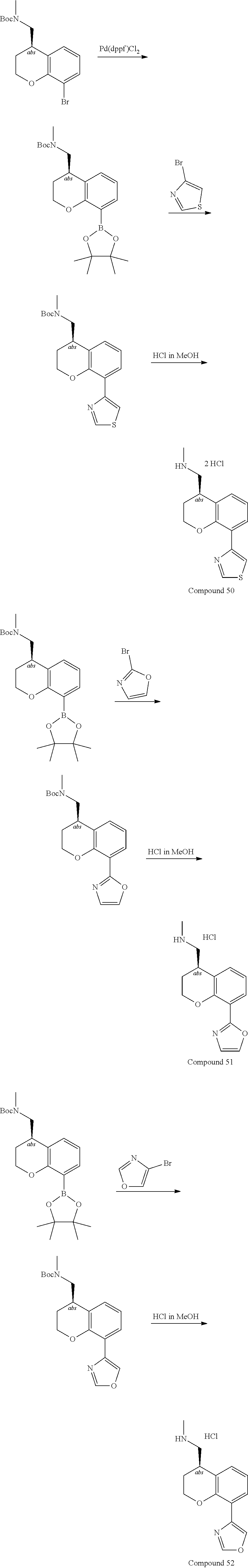

Synthesis of tert-butyl (R)-methyl((8-(4,4,5,5-tetramethyl-1,3,2-dioxaborolan-2-yl)chroman-4-yl)methyl)carbamate: To a solution of tert-butyl (R)-((8-bromochroman-4-yl)methyl)(methyl)carbamate (1.8 g, 5.05 mmol) in dioxane (20 mL) was added [1,1′-bis(diphenylphosphino)ferrocene]dichloropalladium(II) (369 mg, 505 μmol), potassium acetate (1.48 g, 15.1 mmol) and bis(pinacolato)diboron (1.53 g, 6.06 mmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-methyl((8-(4,4,5,5-tetramethyl-1,3,2-dioxaborolan-2-yl)chroman-4-yl)methyl)carbamate (1.0 g, yield: 49%) as a yellow oil. MS (ESI) m/z: 426 [M+Na]⁺.

Synthesis of tert-butyl (R)-methyl((8-(thiazol-4-yl)chroman-4-yl)methyl)carbamate: To a solution of tert-butyl (R)-methyl((8-(4,4,5,5-tetramethyl-1,3,2-dioxaborolan-2-yl)chroman-4-yl)methyl)carbamate (100 mg, 0.248 mmol) in dioxane (1 mL) and water (0.25 mL) was added [1,1′-bis(diphenylphosphino)ferrocene]dichloropalladium(II) (18.1 mg, 24.8 μmol), potassium carbonate (102 mg, 744 μmol) and 4-bromothiazole (48.7 mg, 0.2974 mmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-methyl((8-(thiazol-4-yl)chroman-4-yl)methyl)carbamate (79.2 mg, yield: 89%) as a yellow oil. MS (ESI) m/z: 361 [M+H]⁺.

Synthesis of Compound 50: A solution of tert-butyl (R)-methyl((8-(thiazol-4-yl)chroman-4-yl)methyl)carbamate (70 mg, 0.188 mmol) in HCl in methanol (4 M, 2 mL) was stirred at ambient temperature for 16 h. The reaction was concentrated in vacuum and the residue was triturated with ethyl acetate (10 mL) and the obtained solid was dried by lyophilization to afford Compound 50 (22.4 mg, yield: 35%) as a white solid. (ESI) m/z: 262[M+H]⁺. ¹H NMR (400 MHz, CD₃OD) δ 9.95 (d, J=2.3 Hz, 1H), 8.32 (d, J=2.3 Hz, 1H), 7.69 (dd, J=7.7, 1.4 Hz, 1H), 7.48 (d, J=7.7 Hz, 1H), 7.12 (t, J=7.7 Hz, 1H), 4.50-4.31 (m, 2H), 3.51-3.32 (m, 3H), 2.81 (s, 3H), 2.32-2.22 (m, 1H), 2.19-2.05 (m, 1H).

Synthesis of tert-butyl (R)-methyl((8-(oxazol-2-yl)chroman-4-yl)methyl)carbamate: To a solution of tert-butyl (R)-methyl((8-(4,4,5,5-tetramethyl-1,3,2-dioxaborolan-2-yl)chroman-4-yl)methyl)carbamate (100 mg, 0.248 mmol) in dioxane (1 mL) and water (0.25 mL) was added [1,1′-bis(diphenylphosphino)ferrocene]dichloropalladium(II) (18.1 mg, 24.8 μmol), potassium carbonate (102 mg, 744 μmol) and 2-bromooxazole (43.9 mg, 0.297 mmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-methyl((8-(oxazol-2-yl)chroman-4-yl)methyl)carbamate (70 mg, yield: 82%) as a yellow oil. MS (ESI) m/z: 345 [M+H]⁺.

Synthesis of Compound 51: A solution of tert-butyl (R)-methyl((8-(oxazol-2-yl)chroman-4-yl)methyl)carbamate (70 mg, 0.203 mmol) in HCl in methanol (4 M, 2 mL) was stirred at ambient temperature for 16 h. The reaction was concentrated in vacuum and the residue was triturated with ethyl acetate (10 mL) and the obtained solid was dried by lyophilization to afford Compound 51 (33.4 mg, yield: 59%) as a white solid. (ESI) m/z: 245[M+H]⁺. ¹H NMR (400 MHz, CD₃OD) δ 8.41 (s, 1H), 8.01 (d, J=7.8 Hz, 1H), 7.85 (s, 1H), 7.68 (d, J=7.3 Hz, 1H), 7.21 (t, J 7.8 Hz, 1H), 4.63-4.42 (m, 2H), 3.48-3.34 (m, 3H), 2.81 (s, 3H), 2.36-2.12 (m, 2H).

Synthesis of tert-butyl (R)-methyl((8-(oxazol-4-yl)chroman-4-yl)methyl)carbamate: To a solution of tert-butyl (R)-methyl((8-(4,4,5,5-tetramethyl-1,3,2-dioxaborolan-2-yl)chroman-4-yl)methyl)carbamate (200 mg, 0.496 mmol) in dioxane (1 mL) and water (0.25 mL) was added [1,1′-bis(diphenylphosphino)ferrocene]dichloropalladium(II) (36.2 mg, 496 μmol), potassium carbonate (204 mg, 1.488 mol) and 4-bromooxazole (87.8 mg, 0.594 mmol). The reaction was stirred at 100° C. under nitrogen for 5 h. The reaction was concentrated in vacuum and the residue was purified by flash column chromatography with petroleum ether (100%) to petroleum ether (80%) and ethyl acetate (20%) to provide tert-butyl (R)-methyl((8-(oxazol-4-yl)chroman-4-yl)methyl)carbamate (80 mg, yield: 47%) as a yellow oil. MS (ESI) m/z: 345 [M+H]⁺.

Synthesis of Compound 52: A solution of tert-butyl (R)-methyl((8-(oxazol-4-yl)chroman-4-yl)methyl)carbamate (80 mg, 0.232 mmol) in HCl in Methanol (4 M, 2 mL) was stirred at ambient temperature for 16 h. The reaction was concentrated in vacuum and the residue was triturated with ethyl acetate (10 mL) and the obtained solid was dried by lyophilization to afford Compound 52 (6.4 mg, yield: 10%) as a white solid. (ESI) m/z: 245[M+H]⁺. ¹H NMR (400 MHz, CD₃OD) δ 9.08 (s, 1H), 8.88 (s, 1H), 7.69-7.43 (m, 1H), 7.22 (d, J=6.6 Hz, 1H), 7.11-6.91 (m, 1H), 4.50-4.22 (m, 2H), 3.44-3.32 (m, 3H), 2.79 (s, 3H), 2.34-2.15 (m, 1H), 2.15-1.98 (m, 1H).

### Example 34: Synthesis of Compound 53

### Synthesis of tert-butyl (R)-((8-(4-fluorophenyl)chroman-4-yl)methyl)(methyl)carbamate

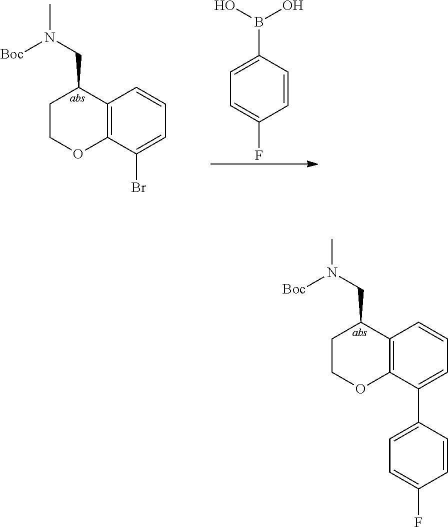 [SMILES: CN(C[C@@H]1CCOc2c(Br)cccc21)C(=O)OC(C)(C)C ; OB(O)c1ccc(F)cc1 ; CN(C[C@@H]1CCOc2c(-c3ccc(F)cc3)cccc21)C(=O)OC(C)(C)C]

To a solution of tert-butyl (R)-((8-bromochroman-4-yl)methyl)(methyl)carbamate (110 mg, 308 μmol) in dioxane (6 mL) and water (3 mL) was added (4-fluorophenyl)boronic acid (43.0 mg, 308 μmol), potassium carbonate (85.1 mg, 616 μmol) and 1,1′-bis(diphenylphosphino)ferrocene-palladium(II) (50.4 mg, 61.6 μmol). The mixture was stirred at 100° C. for 2 hours and concentrated in vacuum. The residue was extracted with ethyl acetate (20 mL). The organic layer was concentrated and purified by silica gel chromatography (petroleum ether/ethyl acetate=3/1) to give tert-butyl (R)-((8-(4-fluorophenyl)chroman-4-yl)methyl)(methyl)carbamate (95 mg, yield: 83.0%)) as a yellow oil. MS (ESI) m/z: 394[M+Na]⁺.

### Synthesis of (R)-1-(8-(4-fluorophenyl)chroman-4-yl)-N-methylmethanamine (Compound 53)

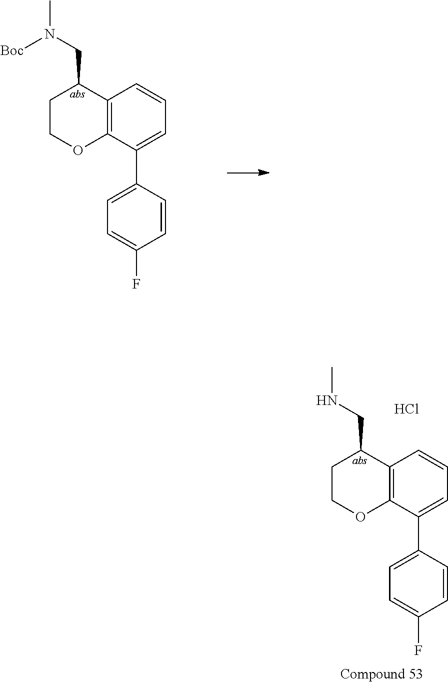 [SMILES: CN(C[C@@H]1CCOc2c(-c3ccc(F)cc3)cccc21)C(=O)OC(C)(C)C ; CNC[C@@H]1CCOc2c(-c3ccc(F)cc3)cccc21 ; Cl]

To a solution of tert-butyl (R)-((8-(4-fluorophenyl)chroman-4-yl)methyl)(methyl) carbamate (90 mg, 242 μmol) in methanol (3 mL) was added HCl/methanol (3 mL, 3 N). The mixture was stirred at room temperature for 3 hours and concentrated. The residue was dried by lyophilization to give Compound 53 (53.5 mg, yield: 71.7%) as a white solid. MS (ESI) m/z: 272[M+H]⁺. NMR: 1H NMR (400 MHz, CD₃OD) δ 7.50-7.40 (m, 2H), 7.25-7.19 (m, 2H), 7.13-7.05 (m, 2H), 6.99 (t, J=7.6 Hz, 1H), 4.31-4.11 (m, 2H), 3.50-3.32 (m, 3H), 2.80 (s, 3H), 2.30-2.12 (m, 1H), 2.06-1.95 (m, 1H).

### Example 35: Synthesis of Compound 54

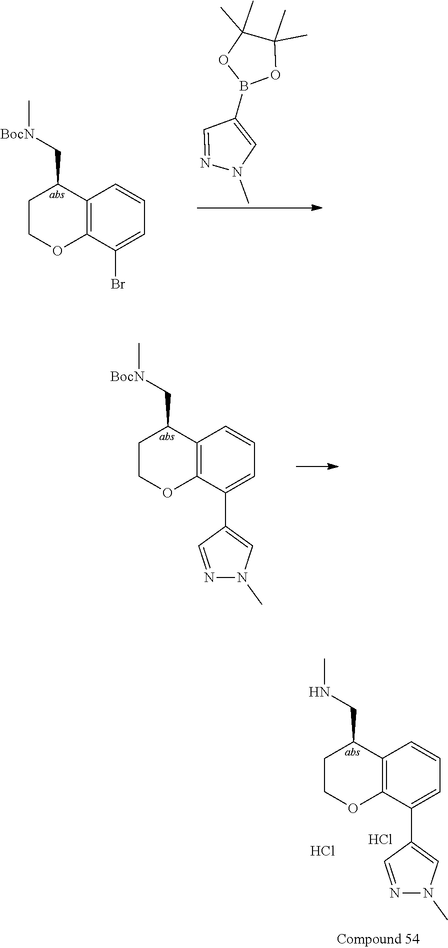 [SMILES: CN(C[C@@H]1CCOc2c(Br)cccc21)C(=O)OC(C)(C)C ; Cn1cc(B2OC(C)(C)C(C)(C)O2)cn1 ; CN(C[C@@H]1CCOc2c(-c3cnn(C)c3)cccc21)C(=O)OC(C)(C)C ; Cl ; CNC[C@@H]1CCOc2c(-c3cnn(C)c3)cccc21 ; Cl]

Synthesis of tert-butyl (R)-methyl((8-(1-methyl-1H-pyrazol-4-yl)chroman-4-yl)methyl)carbamate: To a solution of tert-butyl (R)-((8-bromochroman-4-yl)methyl)(methyl)carbamate (100 mg, 280 μmol) in dioxane (1 mL) and water (0.25 mL) was added [1,1′-bis(diphenylphosphino)ferrocene]dichloropalladium(II) (20.4 mg, 28.0 μmol), potassium carbonate (116 mg, 840 μmol) and 1-methyl-4-(4,4,5,5-tetramethyl-1,3,2-dioxaborolan-2-yl)-1H-pyrazole (75.0 mg, 360 μmol). The reaction was stirred at 100° C. under N₂ for 1 h. The reaction mixture was concentrated and purified by chromatography with a gradient elution of petroleum ether (100%) and ethyl acetate (0%) to petroleum ether (40%) and ethyl acetate (60%) to provide tert-butyl (R)-methyl((8-(1-methyl-1H-pyrazol-4-yl)chroman-4-yl)methyl)carbamate (100 mg, yield: 100%) as a yellow solid. MS (ESI) m/z: 358.3 [M+H]⁺.

Synthesis of (R)—N-methyl-1-(8-(1-methyl-1H-pyrazol-4-yl)chroman-4-yl)methanamine (Compound 54): To a solution of tert-butyl (R)-methyl((8-(1-methyl-1H-pyrazol-4-yl)chroman-4-yl)methyl)carbamate (108 mg, 302 μmol) in HCl (10 mL, 30.0 mmol) was stirred at room temperature for 12 h. The reaction mixture was concentrated and diluted with methanol (0.25 mL), and triturated with ethyl acetate (10 mL) to yield (R)—N-methyl-1-(8-(1-methyl-1H-pyrazol-4-yl)chroman-4-yl)methanamine (38.5 mg, yield: 49.5%) as a white solid. MS (ESI) m/z: 258.2 [M+H]⁺. ¹H NMR (500 MHz, CD3OD) δ 8.49 (s, 1H), 8.44 (s, 1H), 7.54 (d, J=7.6 Hz, 1H), 7.24 (d, J=7.6 Hz, 1H), 7.00 (t, J=7.6 Hz, 1H), 4.52-4.27 (m, 2H), 4.10 (s, 3H), 3.43-3.31 (m, 3H), 2.79 (s, 3H), 2.23 (td, J=9.9, 4.8 Hz, 1H), 2.08 (d, J=14.4 Hz, 1H).

### Example 36: Synthesis of Compound 55

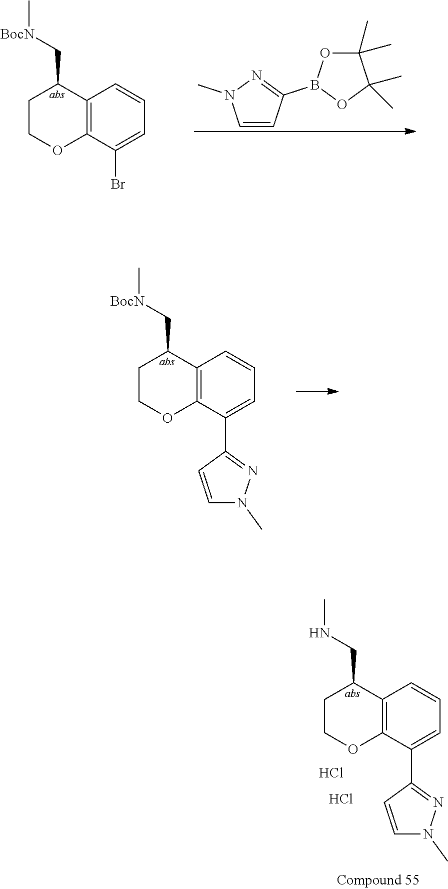

Synthesis of tert-butyl (R)-((8-bromochroman-4-yl)methyl)(methyl)carbamate: To a solution of tert-butyl (R)-((8-bromochroman-4-yl)methyl)(methyl)carbamate (100 mg, 280 mol) in dioxane (1 mL) and water (0.25 mL) was added [1,1′-bis(diphenylphosphino)ferrocene]dichloropalladium(II) (20.4 mg, 28.0 μmol), potassium carbonate (116 mg, 840 μmol) and 1-methyl-3-(4,4,5,5-tetramethyl-1,3,2-dioxaborolan-2-yl)-1H-pyrazole (75.0 mg, 360 μmol). The reaction was stirred at 100° C. under N₂ for 1 h. The reaction mixture was concentrated and purified by chromatography with a gradient elution of petroleum ether (100%) and ethyl acetate (0%) to petroleum ether (40%) and ethyl acetate (60%) to provide tert-butyl (R)-methyl((8-(1-methyl-1H-pyrazol-3-yl)chroman-4-yl)methyl)carbamate (100 mg, yield: 100%) as a yellow solid. MS (ESI) m/z: 358.3 [M+H]⁺.

Synthesis of (R)—N-methyl-1-(8-(1-methyl-1H-pyrazol-3-yl)chroman-4-yl)methanamine: To a solution of tert-butyl (R)-methyl((8-(1-methyl-1H-pyrazol-3-yl)chroman-4-yl)methyl)carbamate (106 mg, 296 μmol) in 3M HCl/methanol (10 mL) was stirred at room temperature for 12 h. The reaction mixture was concentrated and diluted with methanol (0.25 mL), and triturated with ethyl acetate (10 mL) to yield (R)—N-methyl-1-(8-(1-methyl-1H-pyrazol-3-yl)chroman-4-yl)methanamine (Compound 55) (55.9 mg, yield: 73.4%) as a white solid. MS (ESI) m/z: 258.2 [M+H]⁺. ¹H NMR (400 MHz, MeOD) δ 8.16-8.11 (m, 1H), 7.70 (d, J=7.6 Hz, 1H), 7.46 (s, 1H), 7.16-7.00 (m, 2H), 4.56-4.29 (m, 2H), 4.16 (d, J=9.2 Hz, 3H), 3.48-3.31 (m, 3H), 2.80 (s, 3H), 2.34-2.19 (m, 1H), 2.14 (s, 1H).

### Example 37: Synthesis of Compound 56

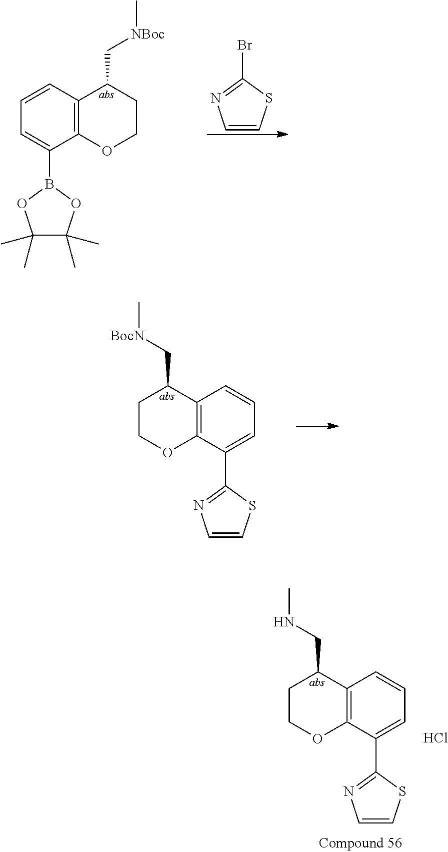 [SMILES: CN(C[C@@H]1CCOc2c(B3OC(C)(C)C(C)(C)O3)cccc21)C(=O)OC(C)(C)C ; Brc1nccs1 ; CN(C[C@@H]1CCOc2c(-c3nccs3)cccc21)C(=O)OC(C)(C)C ; CNC[C@@H]1CCOc2c(-c3nccs3)cccc21 ; Cl]

Synthesis of tert-butyl (R)-methyl((8-(thiazol-2-yl)chroman-4-yl)methyl)carbamate: To a solution of tert-butyl (R)-methyl((8-(4,4,5,5-tetramethyl-1,3,2-dioxaborolan-2-yl)chroman-4-yl)methyl)carbamate (150 mg, 371 μmol) in dioxane (2 mL) and water (0.5 mL) was added [1,1′-bis(diphenylphosphino)ferrocene]dichloropalladium(II) (27.1 mg, 37.1 μmol), potassium carbonate (153 mg, 1.11 mmol) and 2-bromothiazole (90 mg, 548 μmol). The reaction was stirred at 90° C. under nitrogen for 1 h. The reaction mixture was concentrated and purified by chromatography with a gradient elution of petroleum ether (100%) and ethyl acetate (0%) to petroleum ether (40%) and ethyl acetate (60%) to provide tert-butyl (R)-methyl((8-(thiazol-2-yl)chroman-4-yl)methyl)carbamate (96.5 mg, yield: 100%) as a yellow solid. MS (ESI) m/z: 361.2 [M+H]⁺.

Synthesis of (R)—N-methyl-1-(8-(thiazol-2-yl)chroman-4-yl)methanamine: To a solution of tert-butyl (R)-methyl((8-(thiazol-2-yl)chroman-4-yl)methyl)carbamate (100 mg, 277 mol) in 3M HCl/methanol (10 mL) was stirred at room temperature for 12 h. The reaction mixture was concentrated and diluted with methanol (0.25 mL), and triturated with ethyl acetate (10 mL) to yield (R)—N-methyl-1-(8-(thiazol-2-yl)chroman-4-yl)methanamine (Compound 56) (56.6 mg, yield: 78.5%) as a white solid. MS (ESI) m/z: 261.1 [M+H]⁺. 1H NMR (400 MHz, MeOD) δ 8.22 (d, J=3.6 Hz, 1H), 8.09 (dd, J=9.6, 2.6 Hz, 2H), 7.64 (d, J=7.2 Hz, 1H), 7.25 (t, J=8.0 Hz, 1H), 4.70-4.61 (m, 1H), 4.61-4.51 (m, 1H), 3.52-3.36 (m, 3H), 2.84 (s, 3H), 2.40-2.20 (m, 1H), 2.26-2.13 (m, 1H).

### Example 38: Synthesis of Compound 57

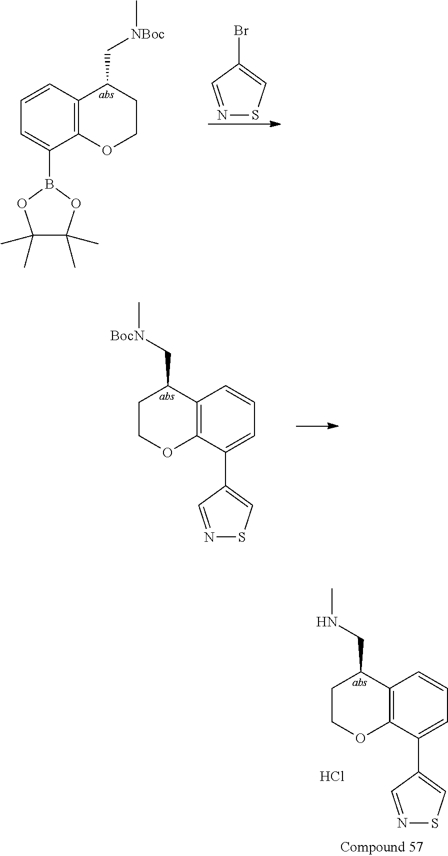 [SMILES: CN(C[C@@H]1CCOc2c(B3OC(C)(C)C(C)(C)O3)cccc21)C(=O)OC(C)(C)C ; Brc1cnsc1 ; CN(C[C@@H]1CCOc2c(-c3cnsc3)cccc21)C(=O)OC(C)(C)C ; Cl ; CNC[C@@H]1CCOc2c(-c3cnsc3)cccc21]

Synthesis of tert-butyl (R)-((8-(isothiazol-4-yl)chroman-4-yl)methyl)(methyl)carbamate: To a solution of tert-butyl (R)-methyl((8-(4,4,5,5-tetramethyl-1,3,2-dioxaborolan-2-yl)chroman-4-yl)methyl)carbamate (150 mg, 371 μmol) in dioxane (2 mL) and water (0.5 mL) was added [1,1′-bis(diphenylphosphino)ferrocene]dichloropalladium(II) (27 mg, 36.8 μmol), potassium carbonate (153 mg, 1.11 mmol) and 4-bromoisothiazole (90 mg, 548 mol). The reaction was stirred at 100° C. under nitrogen for 1 h. The reaction mixture was concentrated and purified by chromatography with a gradient elution of petroleum ether (100%) and ethyl acetate (0%) to petroleum ether (40%) and ethyl acetate (60%) to provide tert-butyl (R)-((8-(isothiazol-4-yl)chroman-4-yl)methyl)(methyl)carbamate (110 mg, yield: 82.7%) as a yellow solid. MS (ESI) m/z: 361.2 [M+H]⁺.

Synthesis of (R)-1-(8-(isothiazol-4-yl)chroman-4-yl)-N-methylmethanamine: To a solution of tert-butyl (R)-((8-(isothiazol-4-yl)chroman-4-yl)methyl)(methyl)carbamate (100 mg, 277 μmol) in 3M HCl/methanol (10 mL) was stirred at room temperature for 12 h. The reaction mixture was concentrated and diluted with methanol (0.25 mL), and triturated with ethyl acetate (10 mL) to yield (R)-1-(8-(isothiazol-4-yl)chroman-4-yl)-N-methylmethanamine (Compound 57) (67 mg, yield: 92.9%) as a white solid. MS (ESI) m/z: 261.1 [M+H]⁺. ¹H NMR (400 MHz, MeOD) δ 9.09 (s, 1H), 8.87 (s, 1H), 7.53 (dd, J=7.6, 1.6 Hz, 1H), 7.28 (dd, J=7.6, 1.3 Hz, 1H), 7.05 (t, J=7.6 Hz, 1H), 4.43-4.26 (m, 2H), 3.50-3.34 (m, 3H), 2.83 (s, 3H), 2.31-2.20 (m, 1H), 2.14-2.01 (m, 1H).

Cellular Assays

Exemplary compounds disclosed herein were tested in functional cell assays for 5-HT_{1A} activity.

### Example 39: 5-HT_{1A} cAMP HTRF Assay (5HT_{1A} Agonism)

CHO-K1 cells expressing the human 5-HT_{1A} receptor (accession number NP_000515.2) were grown in media without antibiotic and detached by gentle flushing with PBS-EDTA (5 mM EDTA), recovered by centrifugation and resuspended in assay buffer (Krebs-Ringers Henseleit buffer: 5 mM KCl, 1.25 mM MgSO₄, 124 mM NaCl, 25 mM HEPES, 13.3 mM Glucose, 1.25 mM KH₂PO₄, 1.45 mM CaCl₂), 0.5 g/L BSA, supplemented with 1 mM 3-Isobutyl-1-Methylxanthine (IBMX).

For agonist testing in 384 well plates, 5 μl of cells (3,000 cells) were mixed with 5 μl of a mix of test compound and forskolin (2 μM final assay concentration) then incubated for 30 minutes at room temperature. cAMP levels were determined using the cAMP Gs dynamic kit (Cisbio Bioassays, 62AM4PEJ). Following the 30-minute incubation, lysis buffer containing cAMP-d2 and anti-cAMP cryptate detection reagents was added, plates were incubated for 1 hour at room temperature, and fluorescence ratios measured according to the manufacturer's protocol. Dose response curves with reference compounds are performed in parallel. Exemplary compounds disclosed herein were tested for 5HT_{1A} agonistic activity.

### Example 40: 5-HT_{1A} Radioligand Binding Assay

Cells expressing the human 5-HT_{1A} receptor were grown to mid-log phase in complete culture medium and then scraped from the culture vessels in ice-cold Ca²⁺- and Mg²⁺-free Phosphate-buffered saline. The cells were then centrifuged for 10 minutes at 5,000×g and 4° C. and the pellets resuspended in buffer A (15 mM Tris-HCl pH 7.5; 2 mM MgCl₂; 0.3 mM EDTA; 1 mM EGTA) and homogenized in a glass-glass homogenizer. The crude membrane fraction was collected by two consecutive centrifugation steps at 35,000×g and 4° C. for 30 minutes separated by a wash step with buffer A. The final membrane pellet was suspended in buffer B (75 mM Tris-HCl pH 7.5; 12.5 mM MgCl₂; 0.3 mM EDTA; 1 mM EGTA; 250 mM sucrose) and flash-frozen in liquid nitrogen. Protein content was determined by the BCA method (Interchim, UP40840A).

Radioligand competition binding was performed in duplicate in the wells of a 96 well plate (Master Block, Greiner, 786201) containing binding buffer (50 mM Tris, 5 mM CaCl₂, 0.1% ascorbic acid, 10 μg/ml saponin, pH7.4), membrane extracts prepared from the 5-HT_{1A} expressing CHO-K1 cell line (7 μg protein/well), reference tracer (0.9 nM [³H]-8-OH-DPAT) and test compound in a final volume of 0.1 ml. Nonspecific binding was determined by co-incubation with 200-fold excess of reference competitor (5-HT). The samples were incubated for 1 hour at 25° C., and then filtered over GF/B Unifilter plates (Perkin Elmer 6005177) presoaked in 0.5% PEI, using a Filtermate Harvester (Perkin Elmer, C96162). Filters were washed six times with 0.5 ml ice-cold buffer (50 mM Tris pH 7.4) and 50 μl of Microscint 20 (Packard) were added to each well. The plates were incubated 15 minutes on an orbital shaker and then counted with a TopCount NXT (Packard, C384V01) for 1 minute/well.

Compounds were tested at 1 and 10 μM concentrations, in duplicate:

Results: The results of the 5HT_{1A} Binding Assay are reported in Table 2. “A” compounds had an inhibition %>90% at 1 μM in the 5HT_{1A} binding Assay; “B” compounds had an inhibition %>50% at 1 μM in the 5HT_{1A} binding Assay.

TABLE 2
Functional Cell Assays for 5-HT_{1A} Agonism

| | 5-HT1_{A} Activity |
|---|---|
| Compound | (% inhibition at 1 μM) |
| 1 | B |
| 2 | A |
| 3 | B |
| 4 | A |

### Example 41: TAAR1 Agonist cAMP Assay Method

CHO-K1 cells expressing the human TAAR1 receptor (Accession number NP_612200.1) or mouse TAAR1 receptor (Accession number NP_444435.1) were grown in media (Advanced DMEM supplemented with 1% dialyzed fetal bovine serum) without antibiotics. The cells were detached by gentle flushing with phosphate buffered saline containing 5 mM EDTA, recovered by centrifugation and resuspended in assay buffer (Krebs-Ringers Henseleit buffer: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/L BSA, supplemented with 1 mM isobutylmethylxanthine). Testing was performed in 384-well plates. 5 μl of cells (3000 cells) were mixed with 5 μl of the test compound diluted in assay buffer and then incubated for 30 minutes at room temperature. cAMP levels were determined using the Cisbio cAMP Gs dynamic HTRF kit (Cisbio, Bedford, MA). The results of the testing are shown in Table 3, with EC₅₀ ranges designated as follows:
A=0.1 μM to 1 μM
B=0.01 μM to <0.1 μM
C=0.001 μM to <0.01 μM
D=0.0001 μM to <0.001 μM

TABLE 3
TAAR1 Agonist cAMP Results

| COMPOUND NO. | TAAR1 EC₅₀ (μM) |
|---|---|
| 3 | B |
| 4 | A |
| 5 | C |
| 6 | C |
| 7 | B |
| 8 | B |
| 9 | D |
| 10 | C |
| 11 | C |
| 12 | A |
| 13 | D |
| 14 | C |
| 15 | C |
| 16 | B |
| 17 | C |
| 18 | C |
| 19 | C |
| 20 | C |
| 21 | B |
| 22 | D |
| 24 | B |
| 25 | B |
| 26 | B |
| 27 | B |
| 28 | B |
| 29 | C |
| 30 | B |
| 31 | C |
| 32 | C |
| 33 | C |
| 34 | B |
| 35 | B |
| 36 | C |
| 37 | B |
| 38 | D |
| 39 | C |
| 40 | C |
| 41 | B |
| 42 | B |
| 43 | A |
| 44 | C |
| 45 | C |
| 48 | C |
| 49 | B |
| 50 | C |
| 53 | C |
| 54 | C |
| 55 | B |
| 56 | B |
| 57 | C |

The teachings of all patents, published applications and references cited herein are incorporated by reference in their entirety.

It may be found upon examination that additional species and genera not presently excluded from the claims to pharmaceutical compositions and chemical compounds are not patentable to the inventors in this application. In that case, the subsequent exclusion of species and genera in applicants' claims are to be considered artifacts of patent prosecution and not reflective of the inventors' concept or description of their invention. The invention, in a composition aspect, is all compounds disclosed herein (e.g., of Table 1) except those that are in the public's possession.

It is to be understood that the invention is not limited to the particular embodiments of the invention described above, as variations of the particular embodiments may be made and still fall within the scope of the appended claims.

Various preferred embodiments [A] to [V] of the invention can be described in the text below:
[Embodiment A] A compound of Formula I

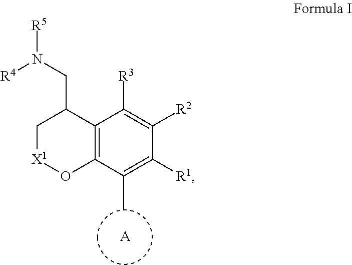

or a pharmaceutically acceptable salt thereof,
   wherein:
   Ring A is (C₅-C₇)aryl or 5 to 7 membered heteroaryl comprising 1 to 3 heteroatoms;
      wherein each (C₅-C₇)aryl or 5 to 7 membered heteroaryl comprising 1 to 3 heteroatoms is independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl;
   X¹ is a bond or CH₂;
   R¹, R², and R³ are each independently H, halogen, (C₁-C₄)alkyl, (C₂-C₄)alkenyl, (C₂-C₄)alkynyl, (C₁-C₄)alkoxy, or (C₁-C₄)haloalkyl; and
   R⁴ and R⁵ are independently H, (C₁-C₄)alkyl, or (C₃-C₆)cycloalkyl.
[Embodiment B] A compound of Embodiment [A] above, or according to other embodiments of the invention, of Formula II(A)

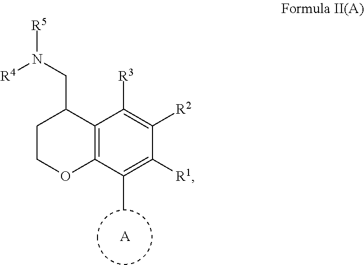

or a pharmaceutically acceptable salt thereof.
[Embodiment C] A compound of Embodiment [A] above, or according to other embodiments of the invention, of Formula III(A)

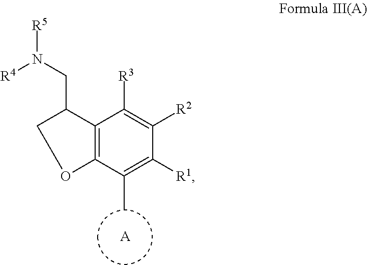

or a pharmaceutically acceptable salt thereof.
[Embodiment D] A compound, or a pharmaceutically acceptable salt thereof, of any one of Embodiments [A] through [C] above, or according to other embodiments of the invention, wherein R⁴ and R⁵ are each independently H or (C₁-C₄)alkyl.
[Embodiment E] A compound, or a pharmaceutically acceptable salt thereof, of any one of Embodiments [A] through [D] above, or according to other embodiments of the invention, wherein R⁴ and R⁵ are each independently H, methyl, or ethyl.
[Embodiment F] A compound, or a pharmaceutically acceptable salt thereof, of any one of Embodiments [A] through [E] above, or according to other embodiments of the invention, wherein one of R⁴ and R⁵ is H.
[Embodiment G] A compound, or a pharmaceutically acceptable salt thereof, of any one of Embodiments [A] through [F] above, or according to other embodiments of the invention, wherein R² and R³ are both H.
[Embodiment H] A compound, or a pharmaceutically acceptable salt thereof, of any one of Embodiments [A] through [G] above, or according to other embodiments of the invention, wherein Ring A is (C₅-C₇)aryl independently optionally substituted with 1 to 3 halogen, (C₁-C₄)alkyl, (C₂-C₄)alkenyl, (C₂-C₄)alkynyl, (C₁-C₄)alkoxy or (C₁-C₄)haloalkyl.
[Embodiment I] A compound, or a pharmaceutically acceptable salt thereof, of any one of Embodiments [A] through [G] above, or according to other embodiments of the invention, wherein Ring A is 5 to 7 membered heteroaryl comprising 1 to 3 heteroatoms independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl.
[Embodiment J] A compound, or a pharmaceutically acceptable salt thereof, of any one of Embodiments [A] through [I] above, or according to other embodiments of the invention, wherein Ring A is unsubstituted.
[Embodiment K] A compound, or a pharmaceutically acceptable salt thereof, of any one of Embodiments [A] through [I] above, or according to other embodiments of the invention, wherein Ring A is substituted with 1 to 3 fluoro, —CH₃, —OCH₃, or —CF₃.
[Embodiment L] A compound, or a pharmaceutically acceptable salt thereof, of any one of Embodiments [A] through [K] above, or according to other embodiments of the invention, wherein R¹, R², and R³ are each independently H.
[Embodiment M] A compound, or a pharmaceutically acceptable salt thereof, of any one of Embodiments [A] through [F] or [H] through [K] above, or according to other embodiments of the invention, wherein R² is H, one of R¹ and R³ is H, and the other of R¹ and R³ is halogen.
[Embodiment N] A compound, or a pharmaceutically acceptable salt thereof, of any one of Embodiments [A] through [F], [H] through [K], or [M] above, or according to other embodiments of the invention, wherein R² is H, one of R¹ and R³ is H, and the other of R¹ and R³ is fluoro.
[Embodiment O] A compound, or a pharmaceutically acceptable salt thereof, of any one of Embodiments [A] through [L] above, or according to other embodiments of the invention, wherein R¹, R², R³, and R⁵ are each independently H; and R⁴ is H or —CH₃.
[Embodiment P] A compound, or a pharmaceutically acceptable salt thereof, of any one of Embodiments [A] through [H], [J] and [L] through [O] above, or according to other embodiments of the invention, wherein Ring A is unsubstituted phenyl.
[Embodiment Q] A compound, or a pharmaceutically acceptable salt thereof, of any one of Embodiments [A] through [H] and [K] through [O] above, or according to other embodiments of the invention, wherein Ring A is phenyl substituted with 1 to 3 fluoro, —CH₃, —OCH₃, or —CF₃.
[Embodiment R] A compound, or a pharmaceutically acceptable salt thereof, of any one of Embodiments [A] through [G] and [I] through [O] above, or according to other embodiments of the invention, wherein Ring A is a 5-membered heteroaryl.
[Embodiment S] A compound, or a pharmaceutically acceptable salt thereof, of any one of Embodiments [A] through [I], [K] through [O], and [Q] through [R] above, or according to other embodiments of the invention, wherein Ring A is substituted with 1 to 3 —CH₃.
[Embodiment T] A compound, or a pharmaceutically acceptable salt thereof, of any one of Embodiments [A] through [S] above, or according to other embodiments of the invention, wherein the compound is shown in Table 1.
[Embodiment U] A compound of any one of Embodiments [A] through [T] above, or according to other embodiments of the invention, wherein the compound is

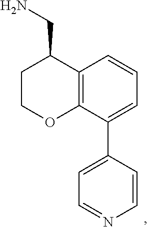 [SMILES: NC[C@@H]1CCOc2c(-c3ccncc3)cccc21]

or a pharmaceutically acceptable salt thereof.
[Embodiment V] A compound of any one of Embodiments [A] through [T] above, or according to other embodiments of the invention, wherein the compound is

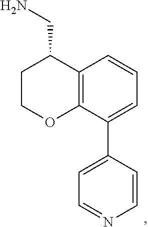 [SMILES: NC[C@H]1CCOc2c(-c3ccncc3)cccc21]

or a pharmaceutically acceptable salt thereof.
[Embodiment W] A compound of any one of Embodiments [A] through [T] above, or according to other embodiments of the invention, wherein the compound is

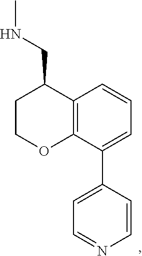 [SMILES: CNC[C@@H]1CCOc2c(-c3ccncc3)cccc21]

or a pharmaceutically acceptable salt thereof.
[Embodiment X] A compound of any one of Embodiments [A] through [T] above or according to other embodiments of the invention, wherein the compound is

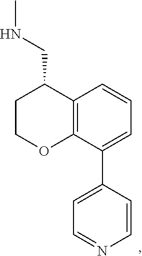 [SMILES: CNC[C@H]1CCOc2c(-c3ccncc3)cccc21]

or a pharmaceutically acceptable salt thereof.
[Embodiment Y] A compound, or a pharmaceutically acceptable salt thereof, of any one of Embodiments [A] through [S] above, or according to other embodiments of the invention, wherein the compound is:

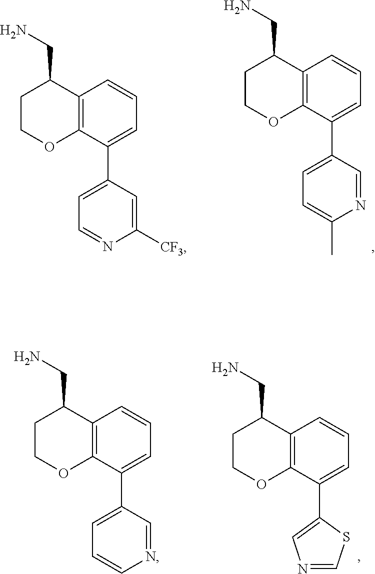 [SMILES: NC[C@@H]1CCOc2c(-c3ccnc(C(F)(F)F)c3)cccc21 ; Cc1ccc(-c2cccc3c2OCC[C@H]3CN)cn1 ; NC[C@@H]1CCOc2c(-c3cccnc3)cccc21 ; NC[C@@H]1CCOc2c(-c3cncs3)cccc21]

or a pharmaceutically acceptable salt thereof.
[Embodiment Z] A compound, or a pharmaceutically acceptable salt thereof, of any one of Embodiments [A] through [S] above, or according to other embodiments of the invention, wherein the compound is:

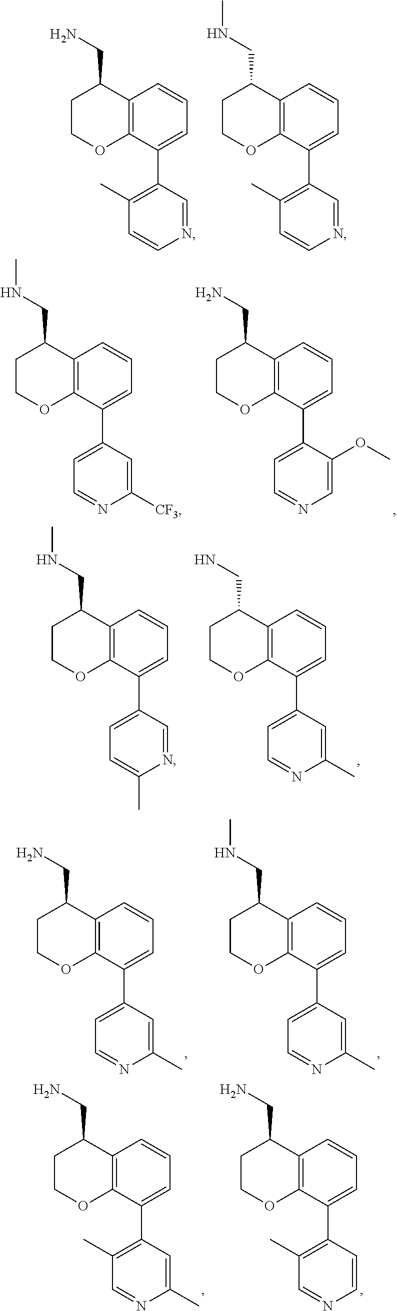

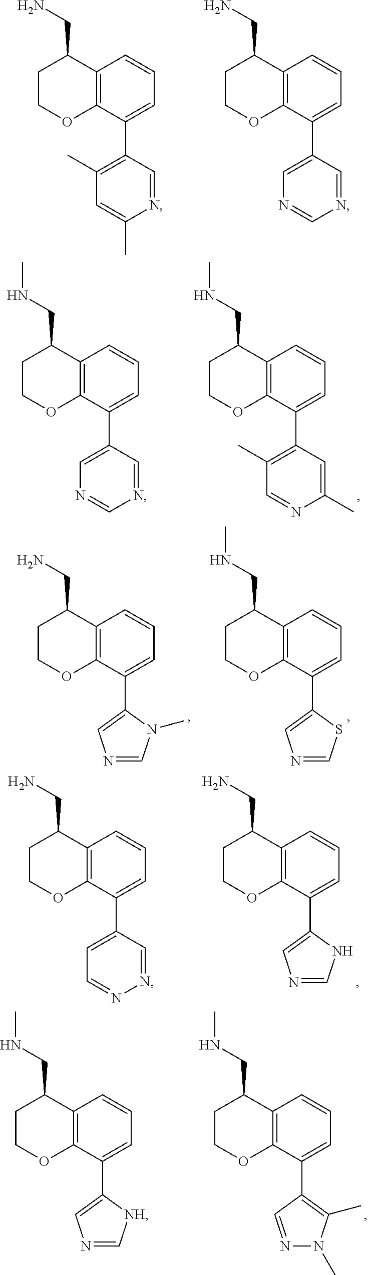

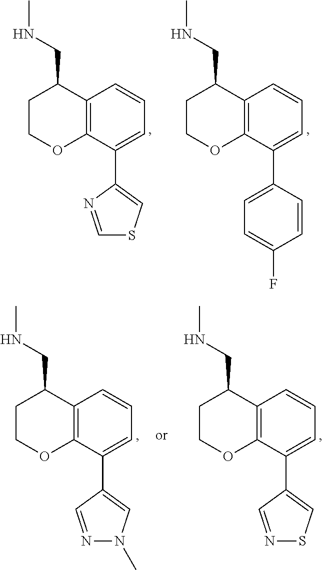

or a pharmaceutically acceptable salt thereof.
[Embodiment AA] A pharmaceutical composition comprising one or more pharmaceutically acceptable excipients and a therapeutically effective amount of a compound, or a pharmaceutically acceptable salt thereof, of any one of Embodiments [A] through [Z] above, or according to other embodiments of the invention.
[Embodiment AB] A pharmaceutical combination comprising a therapeutically effective amount of a compound, or a pharmaceutically acceptable salt thereof, of any one of Embodiments [A] through [Z] above, or according to other embodiments of the invention, and a therapeutically effective amount of one or more other therapeutic agents.
[Embodiment AC] A method of treating a neurological or psychiatric disease or disorder in a subject in need thereof, comprising administering to the subject a therapeutically effective amount of a compound, or a pharmaceutically acceptable salt thereof, of any one of Embodiments [A] through [Z] above, or according to other embodiments of the invention.
[Embodiment AD] A method of treating a neurological or psychiatric disease or disorder in a subject in need thereof, comprising administering to the subject a therapeutically effective amount of a pharmaceutical composition of Embodiment [AA] above, or according to other embodiments of the invention.
[Embodiment AE] A method of treating a neurological or psychiatric disease or disorder in a subject in need thereof, comprising administering to the subject a therapeutically effective amount of a pharmaceutical combination of Embodiment [AB] above, or according to other embodiments of the invention.
[Embodiment AF] The method of any one of Embodiments [AC] through [AE] above, or according to other embodiments of the invention, wherein the neurological or psychiatric disease or disorder is a Movement Disorder, Cognitive Disorder, Pain, Neurodevelopmental Disorder; Schizophrenia Spectrum and Other Psychotic Disorder; Bipolar and Related Disorder; Depressive Disorder; Anxiety Disorder; Obsessive-Compulsive and Related Disorder; Trauma- and Stressor-Related Disorder; Dissociative Disorder; Somatic Symptom and Related Disorder; Feeding and Eating Disorder; Elimination Disorder; Sleep-Wake Disorder; Sexual Dysfunction; Gender Dysphoria; Disruptive, Impulse-Control, and Conduct Disorder; Substance-Related and Addictive Disorder; Neurocognitive Disorder; Personality Disorder; Paraphilic Disorder; Other Mental Disorder; and Medication-Induced Movement Disorder; or Other Adverse Effects of Medication.
[Embodiment AG] The method of any one of Embodiments [AC] through [AF] above, or according to other embodiments of the invention, wherein the neurological or psychiatric disease or disorder is a Depressive Disorder.
[Embodiment AH] The method of any one of Embodiments [AF] through [AG] above, or according to other embodiments of the invention, wherein the Depressive Disorder is a Depression, Disruptive Mood Dysregulation Disorder; Major Depressive Disorder (MDD) (Unipolar Depression); Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; or Unspecified Depressive Disorder.

While example embodiments have been particularly shown and described, it will be understood by those skilled in the art that various changes in form and details may be made therein without departing from the scope of the embodiments encompassed by the appended claims.

1. A compound of Formula I 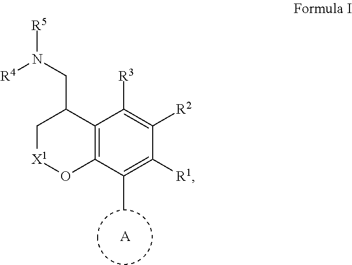
or a pharmaceutically acceptable salt thereof,
wherein:
Ring A is (C₅-C₇)aryl or 5 to 7 membered heteroaryl comprising 1 to 3 heteroatoms;
wherein each (C₅-C₇)aryl or 5 to 7 membered heteroaryl comprising 1 to 3 heteroatoms is independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl;
X¹ is a bond or CH₂;
R¹, R², and R³ are each independently H, halogen, (C₁-C₄)alkyl, (C₂-C₄)alkenyl, (C₂-C₄)alkynyl, (C₁-C₄)alkoxy, or (C₁-C₄)haloalkyl; and
R⁴ and R⁵ are independently H, (C₁-C₄)alkyl, or (C₃—C)cycloalkyl.

2. The compound of claim 1, or a pharmaceutically acceptable salt thereof, having Formula II(A)

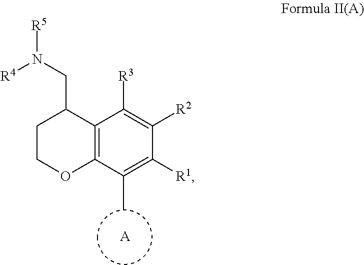

3. The compound of claim 1, or a pharmaceutically acceptable salt thereof, having Formula III(A)

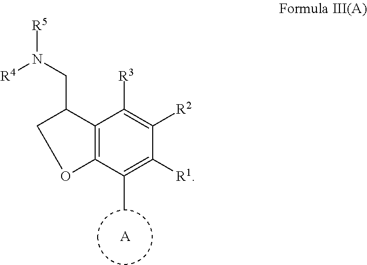

4. The compound of claim 1, or a pharmaceutically acceptable salt thereof, wherein Ring A is (C₅-C₇)aryl independently optionally substituted with 1 to 3 halogen, (C₁-C₄)alkyl, (C₂-C₄)alkenyl, (C₂-C₄)alkynyl, (C₁-C₄)alkoxy or (C₁-C₄)haloalkyl.

5. The compound of claim 1, or a pharmaceutically acceptable salt thereof, wherein Ring A is 5 to 7 membered heteroaryl comprising 1 to 3 heteroatoms independently optionally substituted with 1 to 3 halogen, (C₁-C₄)alkyl, (C₂-C₄)alkenyl, (C₂-C₄)alkynyl, (C₁-C₄)alkoxy, or (C₁-C₄)haloalkyl.

6. The compound of claim 1, or a pharmaceutically acceptable salt thereof, wherein Ring A is unsubstituted or is substituted with 1 to 3 fluoro, —CH₃, —OCH₃, or —CF₃.

7. The compound of claim 1, or a pharmaceutically acceptable salt thereof, wherein R¹, R², and R³ are each independently H.

8. The compound of claim 1, or a pharmaceutically acceptable salt thereof, wherein R¹, R², R³, and R⁵ are each independently H; and R⁴ is H or —CH₃.

9. The compound of claim 1, or a pharmaceutically acceptable salt thereof, wherein Ring A is unsubstituted phenyl or phenyl substituted with 1 to 3 fluoro, —CH₃, —OCH₃, or —CF₃.

10. The compound of claim 1, or a pharmaceutically acceptable salt thereof, wherein Ring A is a 5-membered heteroaryl.

11. The compound of claim 1, or a pharmaceutically acceptable salt thereof, wherein Ring A is unsubstituted or is substituted with 1 to 3 —CH₃.

12. The compound of claim 1 selected from: 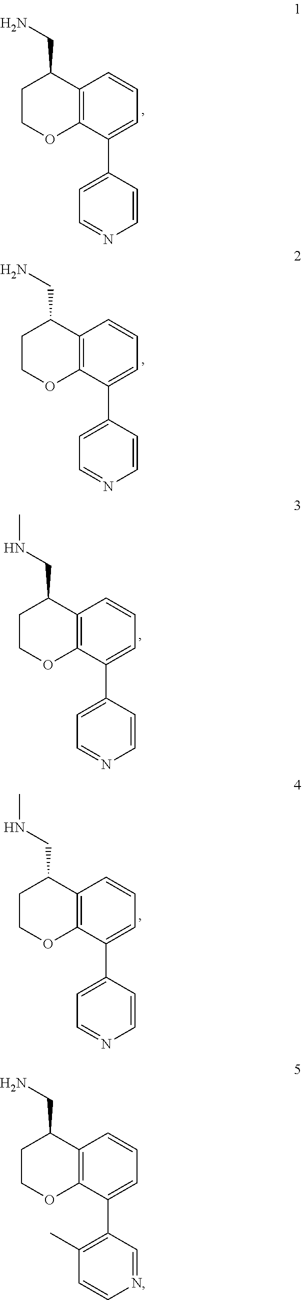 [SMILES: NC[C@@H]1CCOc2c(-c3ccncc3)cccc21 ; NC[C@H]1CCOc2c(-c3ccncc3)cccc21 ; CNC[C@@H]1CCOc2c(-c3ccncc3)cccc21 ; CNC[C@H]1CCOc2c(-c3ccncc3)cccc21 ; Cc1ccncc1-c1cccc2c1OCC[C@H]2CN] 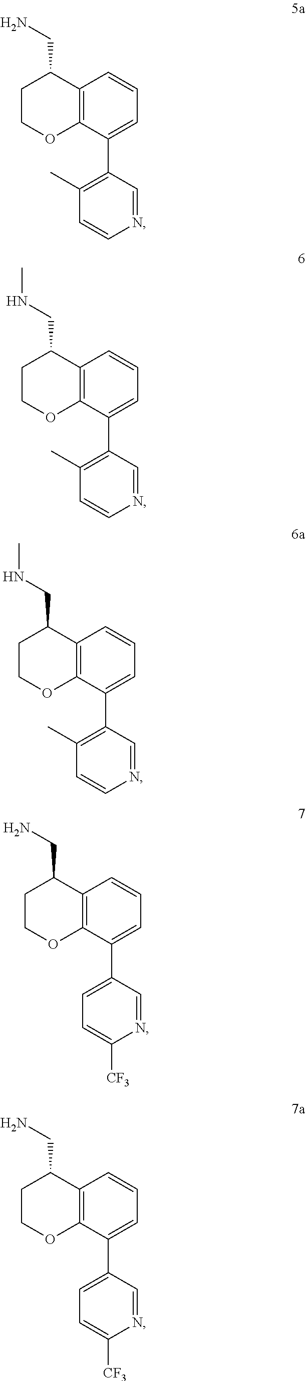 [SMILES: Cc1ccncc1-c1cccc2c1OCC[C@@H]2CN ; CNC[C@H]1CCOc2c(-c3cnccc3C)cccc21 ; CNC[C@@H]1CCOc2c(-c3cnccc3C)cccc21 ; NC[C@@H]1CCOc2c(-c3ccc(C(F)(F)F)nc3)cccc21 ; NC[C@H]1CCOc2c(-c3ccc(C(F)(F)F)nc3)cccc21] 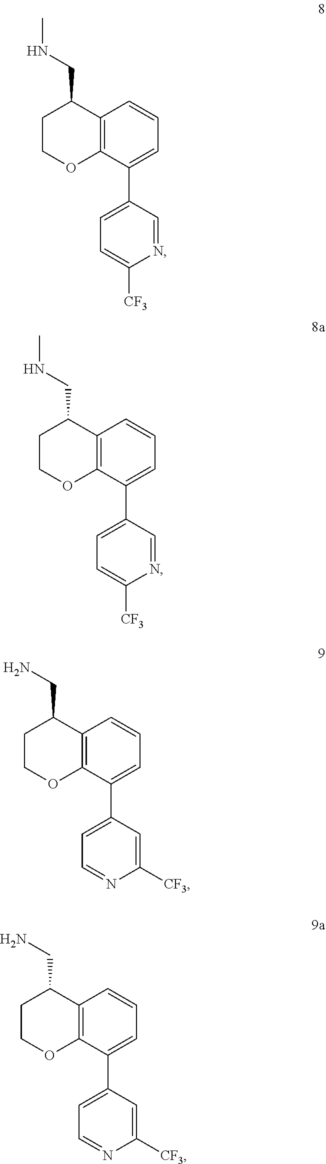 [SMILES: CNC[C@@H]1CCOc2c(-c3ccc(C(F)(F)F)nc3)cccc21 ; CNC[C@H]1CCOc2c(-c3ccc(C(F)(F)F)nc3)cccc21 ; NC[C@@H]1CCOc2c(-c3ccnc(C(F)(F)F)c3)cccc21 ; NC[C@H]1CCOc2c(-c3ccnc(C(F)(F)F)c3)cccc21] 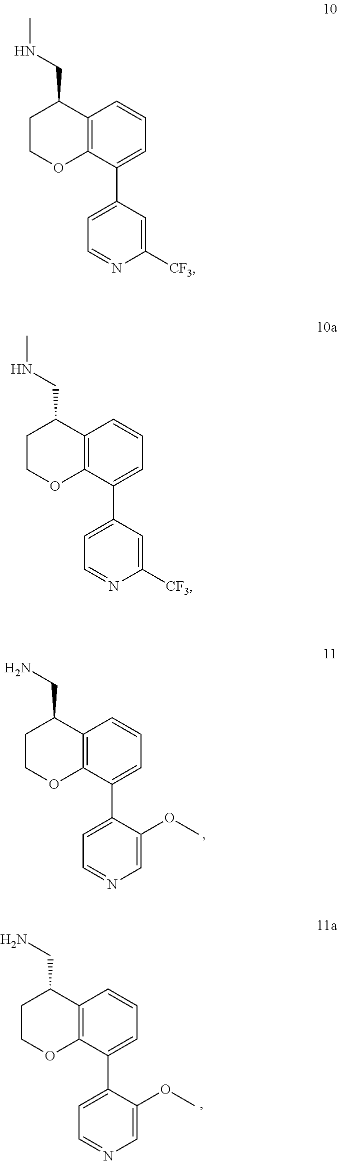 [SMILES: CNC[C@@H]1CCOc2c(-c3ccnc(C(F)(F)F)c3)cccc21 ; CNC[C@H]1CCOc2c(-c3ccnc(C(F)(F)F)c3)cccc21 ; COc1cnccc1-c1cccc2c1OCC[C@H]2CN ; COc1cnccc1-c1cccc2c1OCC[C@@H]2CN] 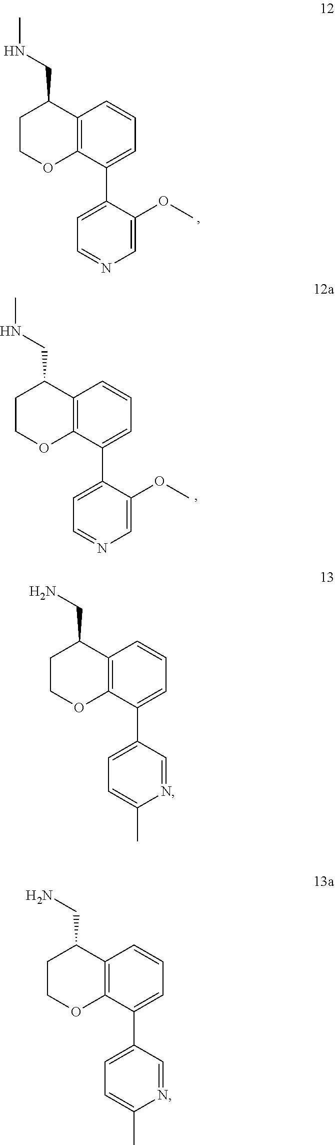 [SMILES: CNC[C@@H]1CCOc2c(-c3ccncc3OC)cccc21 ; CNC[C@H]1CCOc2c(-c3ccncc3OC)cccc21 ; Cc1ccc(-c2cccc3c2OCC[C@H]3CN)cn1 ; Cc1ccc(-c2cccc3c2OCC[C@@H]3CN)cn1] 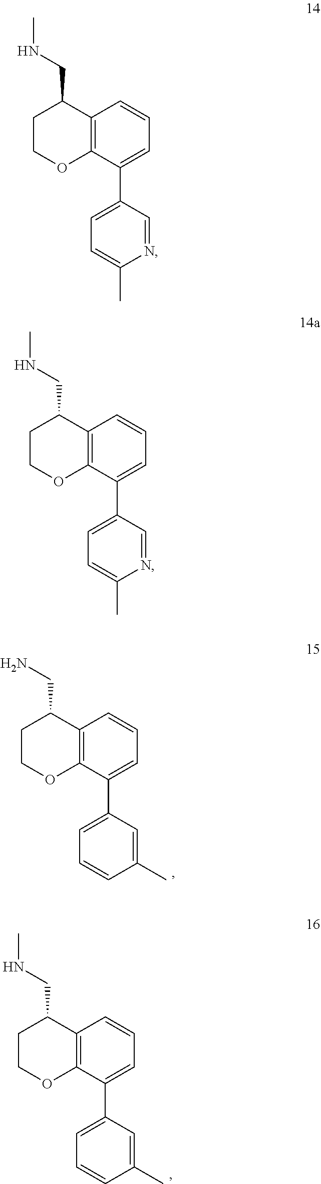 [SMILES: CNC[C@@H]1CCOc2c(-c3ccc(C)nc3)cccc21 ; CNC[C@H]1CCOc2c(-c3ccc(C)nc3)cccc21 ; Cc1cccc(-c2cccc3c2OCC[C@@H]3CN)c1 ; CNC[C@H]1CCOc2c(-c3cccc(C)c3)cccc21] 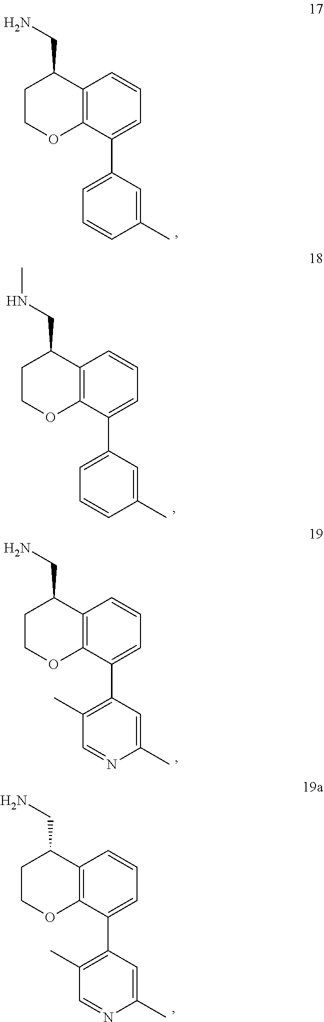 [SMILES: Cc1cccc(-c2cccc3c2OCC[C@H]3CN)c1 ; CNC[C@@H]1CCOc2c(-c3cccc(C)c3)cccc21 ; Cc1cc(-c2cccc3c2OCC[C@H]3CN)c(C)cn1 ; Cc1cc(-c2cccc3c2OCC[C@@H]3CN)c(C)cn1] 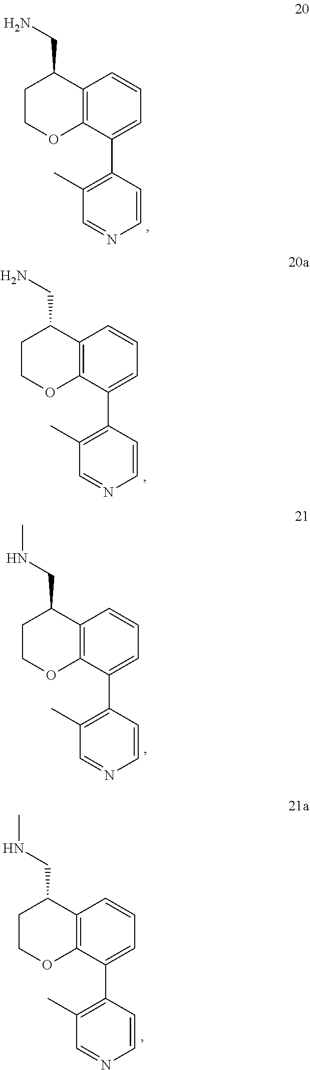 [SMILES: Cc1cnccc1-c1cccc2c1OCC[C@H]2CN ; Cc1cnccc1-c1cccc2c1OCC[C@@H]2CN ; CNC[C@@H]1CCOc2c(-c3ccncc3C)cccc21 ; CNC[C@H]1CCOc2c(-c3ccncc3C)cccc21] 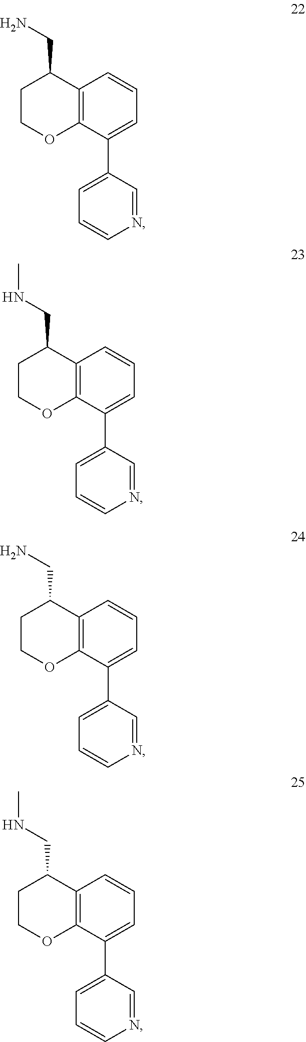 [SMILES: NC[C@@H]1CCOc2c(-c3cccnc3)cccc21 ; CNC[C@@H]1CCOc2c(-c3cccnc3)cccc21 ; NC[C@H]1CCOc2c(-c3cccnc3)cccc21 ; CNC[C@H]1CCOc2c(-c3cccnc3)cccc21] 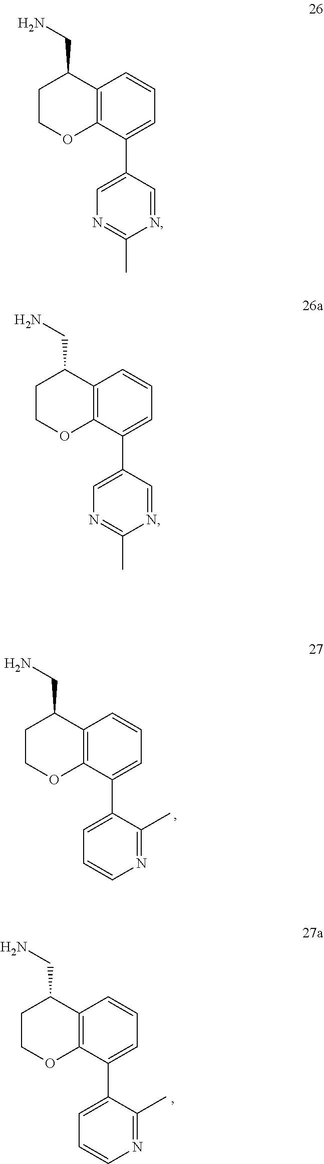 [SMILES: Cc1ncc(-c2cccc3c2OCC[C@H]3CN)cn1 ; Cc1ncc(-c2cccc3c2OCC[C@@H]3CN)cn1 ; Cc1ncccc1-c1cccc2c1OCC[C@H]2CN ; Cc1ncccc1-c1cccc2c1OCC[C@@H]2CN] 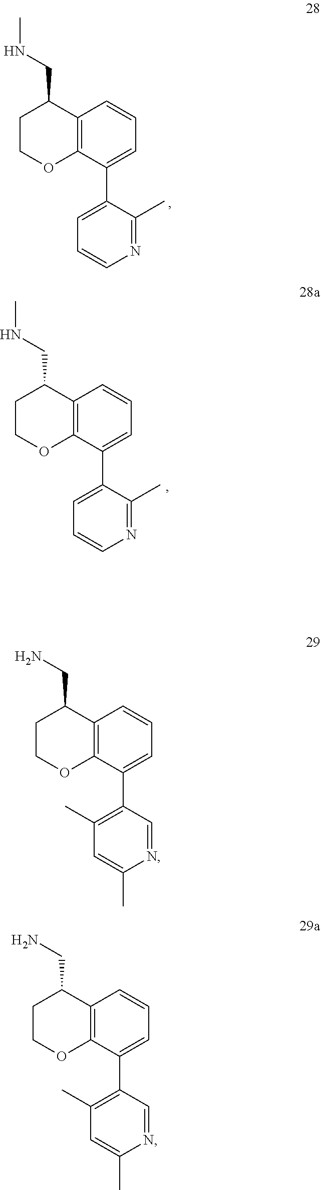 [SMILES: CNC[C@@H]1CCOc2c(-c3cccnc3C)cccc21 ; CNC[C@H]1CCOc2c(-c3cccnc3C)cccc21 ; Cc1cc(C)c(-c2cccc3c2OCC[C@H]3CN)cn1 ; Cc1cc(C)c(-c2cccc3c2OCC[C@@H]3CN)cn1] 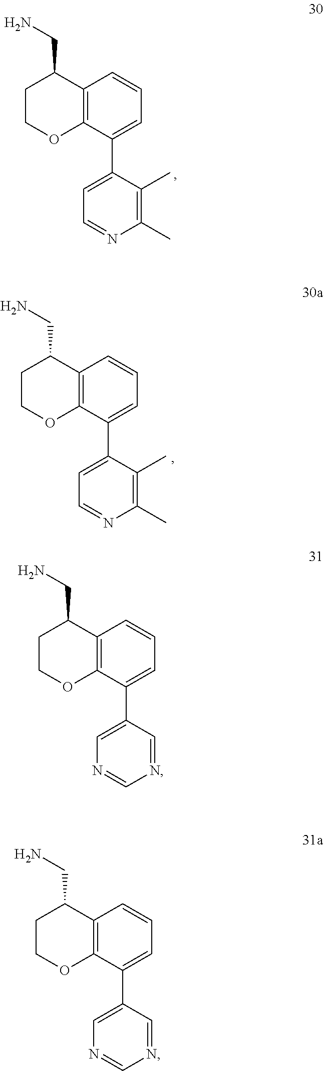 [SMILES: Cc1nccc(-c2cccc3c2OCC[C@H]3CN)c1C ; Cc1nccc(-c2cccc3c2OCC[C@@H]3CN)c1C ; NC[C@@H]1CCOc2c(-c3cncnc3)cccc21 ; NC[C@H]1CCOc2c(-c3cncnc3)cccc21] 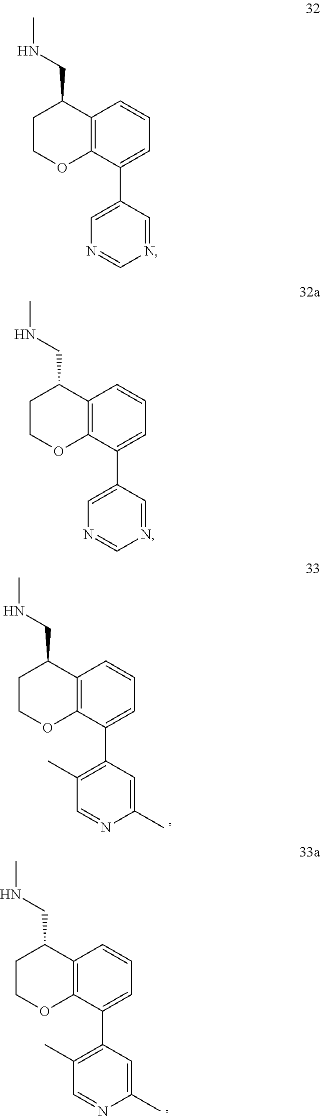 [SMILES: CNC[C@@H]1CCOc2c(-c3cncnc3)cccc21 ; CNC[C@H]1CCOc2c(-c3cncnc3)cccc21 ; CNC[C@@H]1CCOc2c(-c3cc(C)ncc3C)cccc21 ; CNC[C@H]1CCOc2c(-c3cc(C)ncc3C)cccc21] 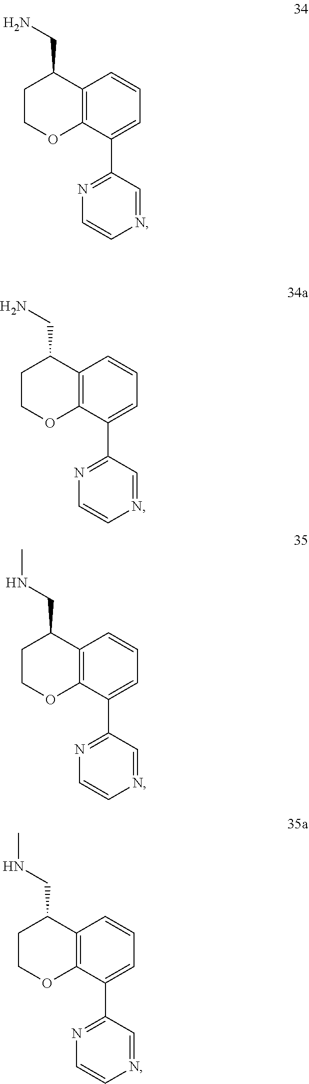 [SMILES: NC[C@@H]1CCOc2c(-c3cnccn3)cccc21 ; NC[C@H]1CCOc2c(-c3cnccn3)cccc21 ; CNC[C@@H]1CCOc2c(-c3cnccn3)cccc21 ; CNC[C@H]1CCOc2c(-c3cnccn3)cccc21] 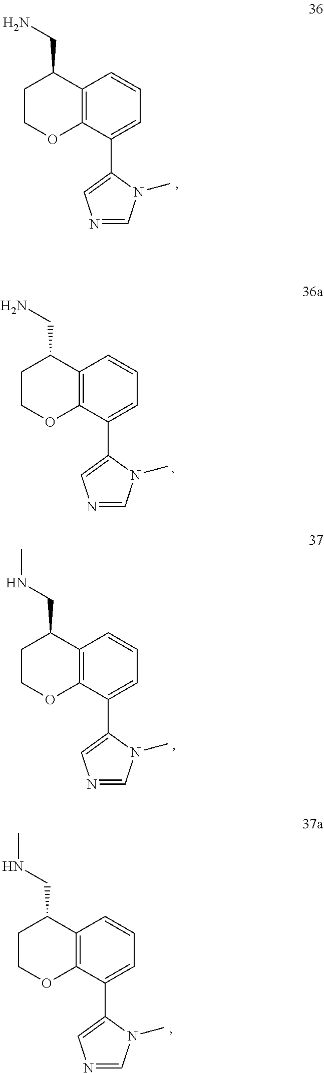 [SMILES: Cn1cncc1-c1cccc2c1OCC[C@H]2CN ; Cn1cncc1-c1cccc2c1OCC[C@@H]2CN ; CNC[C@@H]1CCOc2c(-c3cncn3C)cccc21 ; CNC[C@H]1CCOc2c(-c3cncn3C)cccc21] 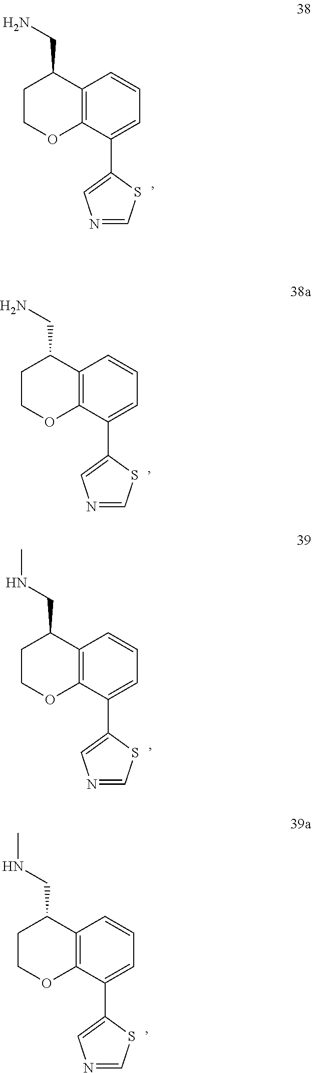 [SMILES: NC[C@@H]1CCOc2c(-c3cncs3)cccc21 ; NC[C@H]1CCOc2c(-c3cncs3)cccc21 ; CNC[C@@H]1CCOc2c(-c3cncs3)cccc21 ; CNC[C@H]1CCOc2c(-c3cncs3)cccc21] 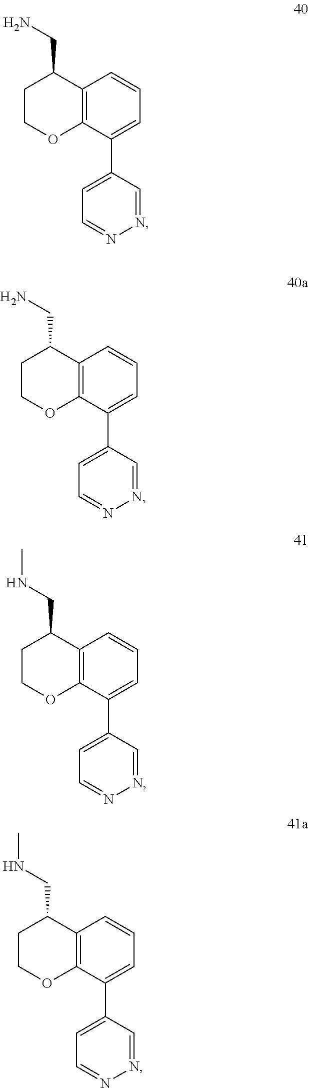 [SMILES: NC[C@@H]1CCOc2c(-c3ccnnc3)cccc21 ; NC[C@H]1CCOc2c(-c3ccnnc3)cccc21 ; CNC[C@@H]1CCOc2c(-c3ccnnc3)cccc21 ; CNC[C@H]1CCOc2c(-c3ccnnc3)cccc21] 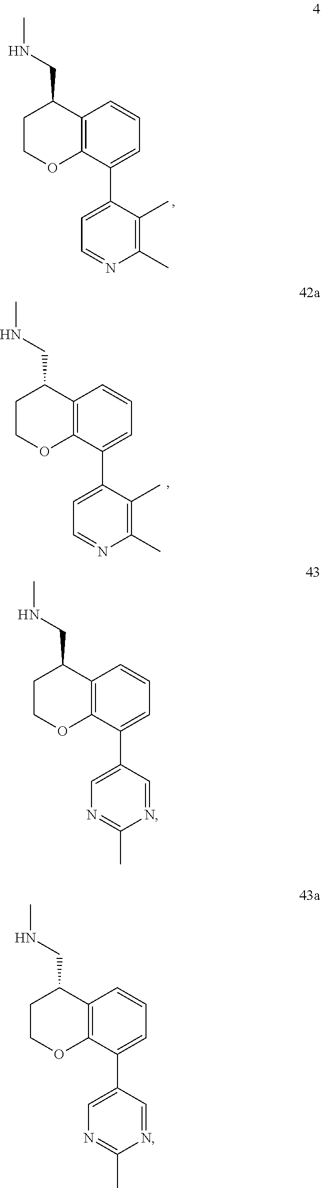 [SMILES: CNC[C@@H]1CCOc2c(-c3ccnc(C)c3C)cccc21 ; CNC[C@H]1CCOc2c(-c3ccnc(C)c3C)cccc21 ; CNC[C@@H]1CCOc2c(-c3cnc(C)nc3)cccc21 ; CNC[C@H]1CCOc2c(-c3cnc(C)nc3)cccc21] 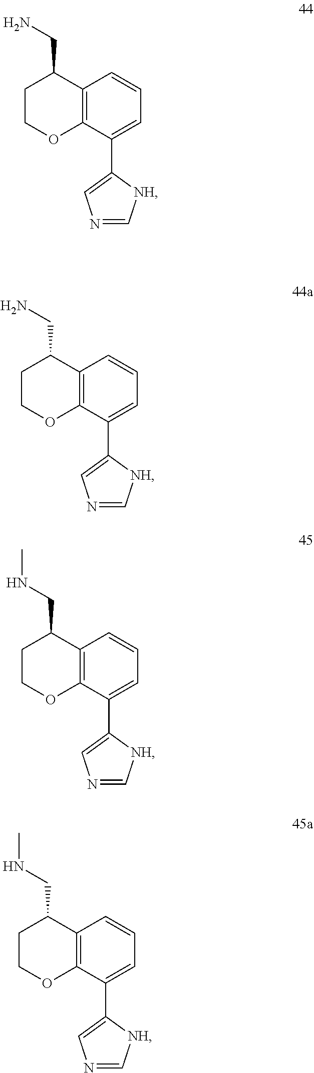 [SMILES: NC[C@@H]1CCOc2c(-c3cnc[nH]3)cccc21 ; NC[C@H]1CCOc2c(-c3cnc[nH]3)cccc21 ; CNC[C@@H]1CCOc2c(-c3cnc[nH]3)cccc21 ; CNC[C@H]1CCOc2c(-c3cnc[nH]3)cccc21] 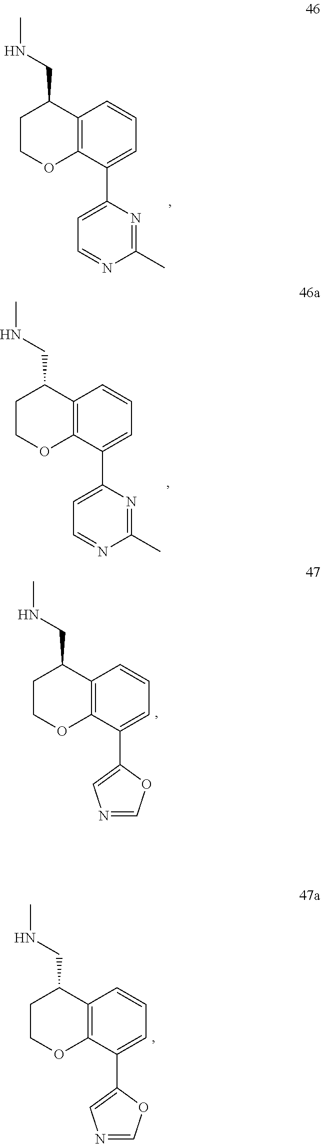 [SMILES: CNC[C@@H]1CCOc2c(-c3ccnc(C)n3)cccc21 ; CNC[C@H]1CCOc2c(-c3ccnc(C)n3)cccc21 ; CNC[C@@H]1CCOc2c(-c3cnco3)cccc21 ; CNC[C@H]1CCOc2c(-c3cnco3)cccc21] 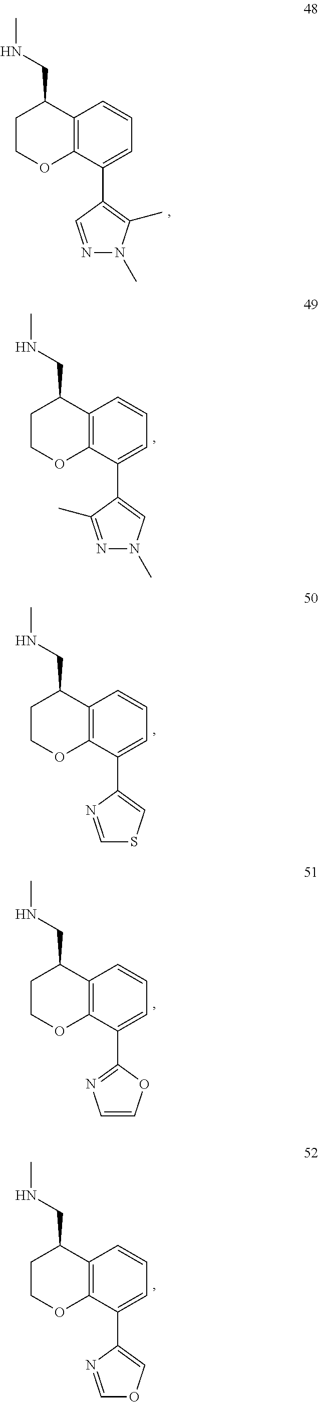 [SMILES: CNC[C@@H]1CCOc2c(-c3cnn(C)c3C)cccc21 ; CNC[C@@H]1CCOc2c(-c3cn(C)nc3C)cccc21 ; CNC[C@@H]1CCOc2c(-c3cscn3)cccc21 ; CNC[C@@H]1CCOc2c(-c3ncco3)cccc21 ; CNC[C@@H]1CCOc2c(-c3cocn3)cccc21] 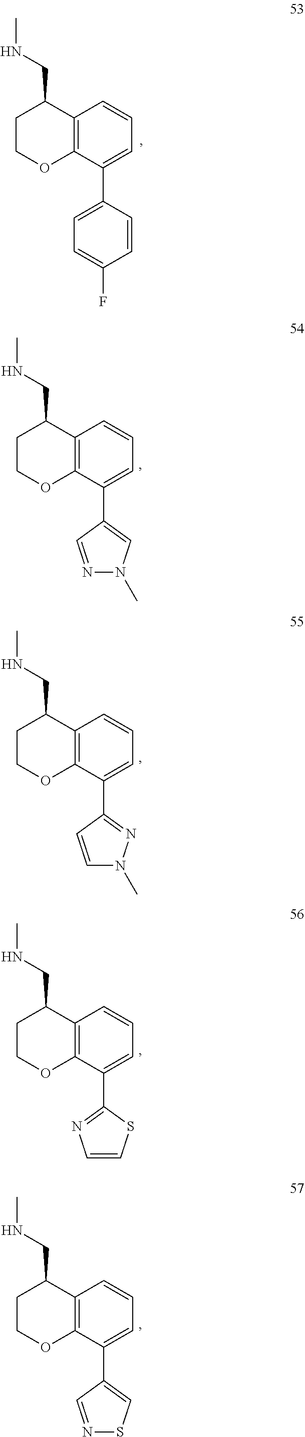 [SMILES: CNC[C@@H]1CCOc2c(-c3ccc(F)cc3)cccc21 ; CNC[C@@H]1CCOc2c(-c3cnn(C)c3)cccc21 ; CNC[C@@H]1CCOc2c(-c3ccn(C)n3)cccc21 ; CNC[C@@H]1CCOc2c(-c3nccs3)cccc21 ; CNC[C@@H]1CCOc2c(-c3cnsc3)cccc21] 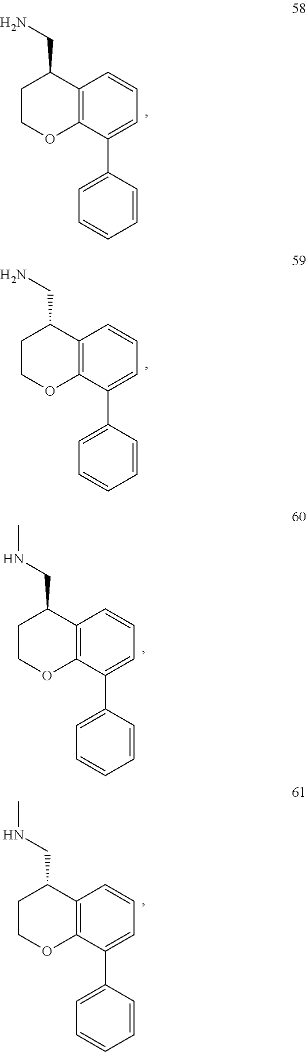 [SMILES: NC[C@@H]1CCOc2c(-c3ccccc3)cccc21 ; NC[C@H]1CCOc2c(-c3ccccc3)cccc21 ; CNC[C@@H]1CCOc2c(-c3ccccc3)cccc21 ; CNC[C@H]1CCOc2c(-c3ccccc3)cccc21] 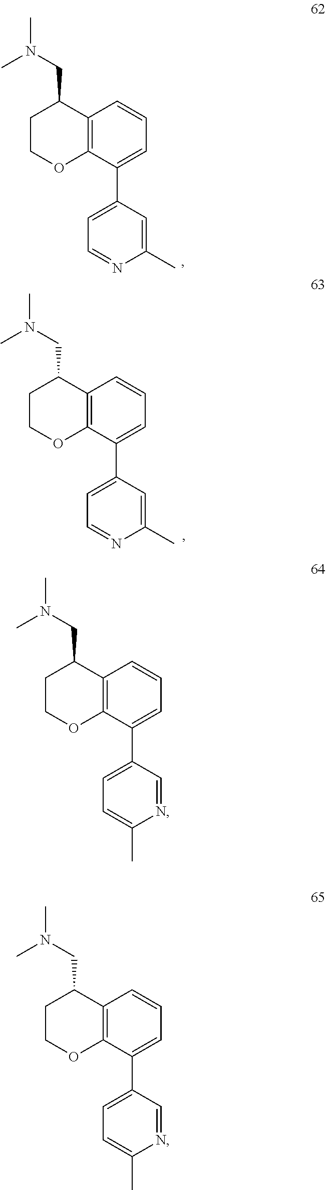 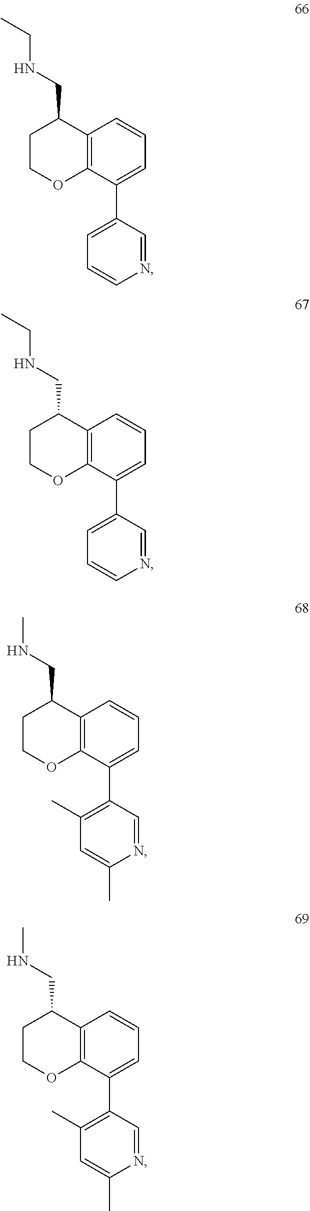 [SMILES: CCNC[C@@H]1CCOc2c(-c3cccnc3)cccc21 ; CCNC[C@H]1CCOc2c(-c3cccnc3)cccc21 ; CNC[C@@H]1CCOc2c(-c3cnc(C)cc3C)cccc21 ; CNC[C@H]1CCOc2c(-c3cnc(C)cc3C)cccc21] 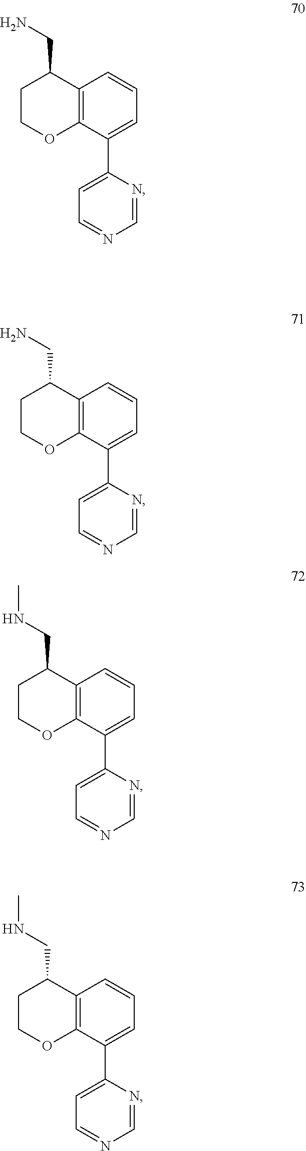 [SMILES: NC[C@@H]1CCOc2c(-c3ccncn3)cccc21 ; NC[C@H]1CCOc2c(-c3ccncn3)cccc21 ; CNC[C@@H]1CCOc2c(-c3ccncn3)cccc21 ; CNC[C@H]1CCOc2c(-c3ccncn3)cccc21] 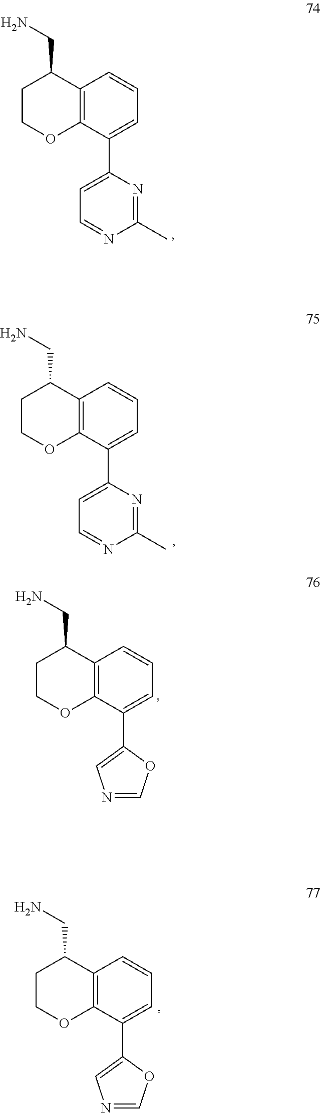 [SMILES: Cc1nccc(-c2cccc3c2OCC[C@H]3CN)n1 ; Cc1nccc(-c2cccc3c2OCC[C@@H]3CN)n1 ; NC[C@@H]1CCOc2c(-c3cnco3)cccc21 ; NC[C@H]1CCOc2c(-c3cnco3)cccc21] 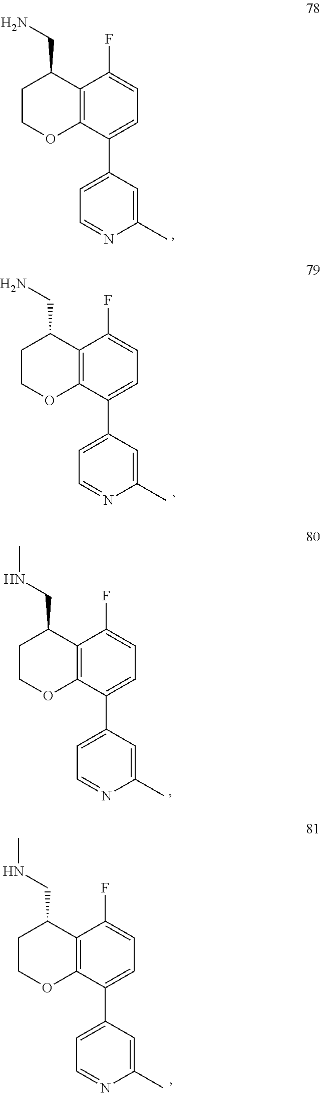 [SMILES: Cc1cc(-c2ccc(F)c3c2OCC[C@H]3CN)ccn1 ; Cc1cc(-c2ccc(F)c3c2OCC[C@@H]3CN)ccn1 ; CNC[C@@H]1CCOc2c(-c3ccnc(C)c3)ccc(F)c21 ; CNC[C@H]1CCOc2c(-c3ccnc(C)c3)ccc(F)c21] 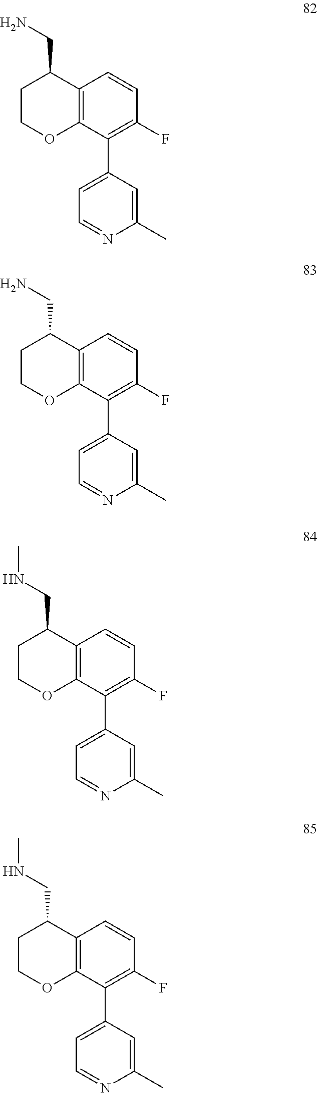 [SMILES: Cc1cc(-c2c(F)ccc3c2OCC[C@H]3CN)ccn1 ; Cc1cc(-c2c(F)ccc3c2OCC[C@@H]3CN)ccn1 ; CNC[C@@H]1CCOc2c1ccc(F)c2-c1ccnc(C)c1 ; CNC[C@H]1CCOc2c1ccc(F)c2-c1ccnc(C)c1] 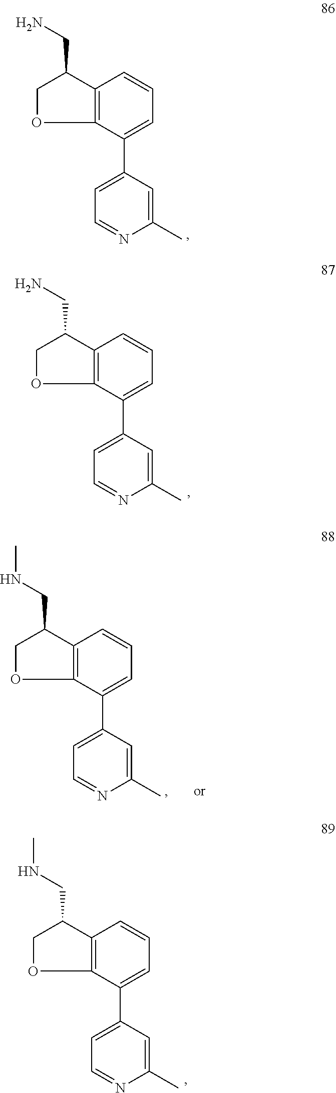 [SMILES: Cc1cc(-c2cccc3c2OC[C@H]3CN)ccn1 ; Cc1cc(-c2cccc3c2OC[C@@H]3CN)ccn1 ; CNC[C@@H]1COc2c(-c3ccnc(C)c3)cccc21 ; CNC[C@H]1COc2c(-c3ccnc(C)c3)cccc21]
or a pharmaceutically acceptable salt thereof.

13. The compound of claim 12 selected from 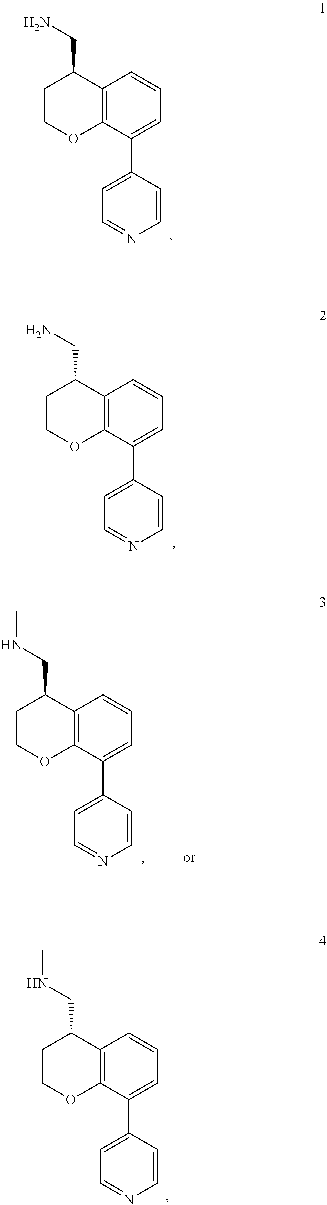 [SMILES: NC[C@@H]1CCOc2c(-c3ccncc3)cccc21 ; NC[C@H]1CCOc2c(-c3ccncc3)cccc21 ; CNC[C@@H]1CCOc2c(-c3ccncc3)cccc21 ; CNC[C@H]1CCOc2c(-c3ccncc3)cccc21]
or a pharmaceutically acceptable salt thereof.

14. A pharmaceutical composition, comprising a therapeutically effective amount of a compound of claim 1, or a pharmaceutically acceptable salt thereof, and one or more pharmaceutically acceptable excipient.

15. A pharmaceutical combination, comprising a therapeutically effective amount of a compound of claim 1, or a pharmaceutically acceptable salt thereof, and a therapeutically effective amount of one or more other therapeutic agents.

16. A method of treating a neurological or psychiatric disease or disorder in a subject in need thereof, comprising administering to the subject a therapeutically effective amount of a compound according to claim 1, or a pharmaceutically acceptable salt thereof.

17. The method of claim 16 wherein the neurological or psychiatric disease or disorder is a Movement Disorder, Cognitive Disorder, Pain, Neurodevelopmental Disorder; Schizophrenia Spectrum and Other Psychotic Disorder; Bipolar and Related Disorder; Depressive Disorder; Anxiety Disorder; Obsessive-Compulsive and Related Disorder; Trauma- and Stressor-Related Disorder; Dissociative Disorder; Somatic Symptom and Related Disorder; Feeding and Eating Disorder; Elimination Disorder; Sleep-Wake Disorder; Sexual Dysfunction; Gender Dysphoria; Disruptive, Impulse-Control, and Conduct Disorder; Substance-Related and Addictive Disorder; Neurocognitive Disorder; Personality Disorder; Paraphilic Disorder; Other Mental Disorder; and Medication-Induced Movement Disorder; or Other Adverse Effects of Medication.

18. The method of claim 17 wherein the neurological or psychiatric disease or disorder is a Depressive Disorder.

19. The method of claim 18 wherein the Depressive Disorder is a Depression, Disruptive Mood Dysregulation Disorder; Major Depressive Disorder (MDD) (Unipolar Depression); Persistent Depressive Disorder (Dysthymia); Premenstrual Dysphoric Disorder; Substance/Medication-Induced Depressive Disorder; Treatment-Resistant Depression; Depressive Disorder Due to Another Medical Condition; Other Specified Depressive Disorder; or Unspecified Depressive Disorder.
